(19) United States
(12) Patent Application Publication
(10) Pub. No.: US 2014/0106448 A1
(43) Pub. Date: Apr. 17, 2014

(54) METHODS OF ISOLATING CELLS

(71) Applicant: TissueTech, Inc., Doral, FL (US)

(72) Inventors: Szu-yu Chen, Miami, FL (US); Scheffer Tseng, Pinecrest, FL (US)

(73) Assignee: TissueTech, Inc., Doral, FL (US)

(21) Appl. No.: 14/004,996

(22) PCT Filed: Apr. 30, 2012
(86) PCT No.: PCT/US12/35897

§ 371 Date: Dec. 20, 2013

Related U.S. Application Data
(60) Provisional application No. 61/481,050, filed on Apr. 29, 2011.

(51) Int. Cl. C12N 5/074

(52) U.S. Cl. CPC C12N 5/0607
USPC 435/366; 435/325; 435/408

(57) ABSTRACT

Provided herein are methods of isolating and expanding a plurality of multipotent cells (e.g., MSCs and ASCs) using a first culture in a suitable 2-dimensional substrate and a second culture in a suitable 3-dimensional substrate containing the basement membrane component and ROCK inhibitors. Also described are multipotent cell cultures made by the methods. Multipotent cell cultures as described may be used for transplantation or as niche cells for supporting other types of stem cells.

### CROSS-REFERENCE

This application claims the benefit of U.S. Provisional Application No. 61/481,050, filed Apr. 29, 2011, which application is incorporated herein by reference.

### STATEMENT AS TO FEDERALLY SPONSORED RESEARCH

This invention was made with government support under RO1EY06819 awarded by the National Eye Institute. The government has certain rights in the invention.

### BACKGROUND OF THE INVENTION

Human mesenchymal stromal cells (MSC) have been shown to differentiate into multiple mesoderm-type lineages, including chondrocytes, osteoblasts, and adipocytes and into ectodermal and endodermal origin. MSC have been isolated from several tissues including bone marrow, peripheral blood, adipose tissue, liver, skin, amniotic fluid, placenta and umbilical cord.

### SUMMARY OF THE INVENTION

Disclosed herein, in certain embodiments, are methods of isolating and expanding a plurality of multipotent cells, comprising: (a) separating a plurality of multipotent cells from other bound cells and components of an extracellular matrix in a tissue sample, to form a plurality of isolated multipotent cells; (b) expanding at least one of the plurality of isolated multipotent cells in a first culture comprising a suitable 2-dimensional substrate without passing the Hayflick limit, to form a plurality of expanding multipotent cells; and (c) isolating and expanding at least one multipotent cell from the plurality of expanding multipotent cells in a second culture comprising a suitable 3-dimensional substrate, to generate a population of expanded multipotent cells. In some embodiments, the multipotent cells are mesenchymal stromal cells (MSC) and/or adipose derived stromal cells (ASC). In some embodiments, the 2-dimensional substrate mimics the extracellular environment found in tissues. In some embodiments, the 2-dimensional substrate comprises a gelatinous protein mixture secreted by Engelbreth-Holm-Swarm (EHS) mouse sarcoma cells. In some embodiments, the 2-dimensional substrate comprises as laminin, type IV collagen and heparan sulfate proteoglycans. In some embodiments, the 3-dimensional substrate mimics the extracellular environment found in tissues. In some embodiments, the 3-dimensional substrate comprises a gelatinous protein mixture secreted by Engelbreth-Holm-Swarm (EHS) mouse sarcoma cells. In some embodiments, the 3-dimensional substrate comprises as laminin, type IV collagen and heparan sulfate proteoglycans. In some embodiments, the first culture further comprises an embryonic stem cell medium, supplemented hormonal epithelial medium, and/or a combination thereof. In some embodiments, the first culture comprises an embryonic stem cell medium. In some embodiments, the embryonic stem cell medium is a human embryonic stem cell medium. In some embodiments, the embryonic stem cell medium comprises bFGF and/or LIF. In some embodiments, the second culture comprises an embryonic stem cell medium, supplemented hormonal epithelial medium, and/or a combination thereof. In some embodiments, the first culture comprises an embryonic stem cell medium. In some embodiments, the embryonic stem cell medium is a human embryonic stem cell medium. In some embodiments, the embryonic stem cell medium comprises bFGF and/or LIF. In some embodiments, the first or second culture further comprises an inhibitor of Rho-associated kinase. In some embodiments, the tissue sample comprises stroma and/or epithelium. In some embodiments, the tissue sample is placenta, umbilical cord, chorion, limbal tissue, conjunctiva, the skin, the oral mucosa, adipose tissue and/or a combination thereof. In some embodiments, the multipotent cells are separated from other bound cells and components of an extracellular matrix (but not the basement membrane) in the tissue sample by contacting the tissue sample with an enzyme that degrades interstitial components of the extracellular matrix but not basement membrane components. In some embodiments, the multipotent cells are separated from other bound cells and components of an extracellular matrix (but not the basement membrane) in the tissue sample by contacting the tissue sample with an enzyme that degrades interstitial collagen but not basement membrane collagen. In some embodiments, the multipotent cells are separated from other bound cells and components of an extracellular matrix (but not the basement membrane) in the tissue sample by contacting the tissue sample with dispase. In some embodiments, the multipotent cells are separated from other bound cells and components of an extracellular matrix (but not the basement membrane) in the tissue sample by contacting the tissue sample with a collagenase. In some embodiments, the multipotent cells are separated from other bound cells and components of an extracellular matrix (but not the basement membrane) in the tissue sample by contacting the tissue sample with collagenase A. In some embodiments, the multipotent cells are separated from other bound cells and components of an extracellular matrix (but not the basement membrane) in the tissue sample by contacting the tissue sample with dispase and collagenase A.

Disclosed herein, in certain embodiments, are methods of expanding a plurality of multipotent cells, comprising: expanding at least one isolated multipotent cell in a culture comprising a suitable coated and/or 2-dimensional substrate without passing the Hayflick limit, to form a plurality of expanding multipotent cells, wherein the 2D substrate comprises a gelatinous protein mixture secreted by Engelbreth-Holm-Swarm (EHS) mouse sarcoma cells. In some embodiments, the methods further comprise expanding at least one expanding multipotent cell in a culture comprising a suitable 3-dimensional substrate, to generate a population of expanded multipotent cells, wherein the 3D substrate comprises a gelatinous protein mixture secreted by Engelbreth-Holm-Swarm (EHS) mouse sarcoma cells. In some embodiments, the methods contacting a tissue sample comprising a plurality of multipotent cells with a collagenase, to form a plurality of isolated multipotent cells. In some embodiments, the multipotent cells are mesenchymal stromal cells (MSC) and/or adipose derived stromal cells (ASC). In some embodiments, the coated and/or 2-dimensional substrate mimics the extracellular environment found in tissues. In some embodiments, the 3-dimensional substrate mimics the extracellular environment found in tissues. In some embodiments, the culture comprising the suitable 3-dimensional substrate further comprises an embryonic stem cell medium, supplemented hormonal epithelial medium, and/or a combination thereof. In some embodiments, the culture comprising the suitable 3-dimensional substrate further comprises an embryonic stem cell medium. In some embodiments, the culture comprising the suitable 3-dimensional substrate further comprises an embryonic stem cell medium supplemented with bFGF and/or LIF. In some embodiments, the culture comprising the suitable coated and/or 2-dimensional substrate further comprises an embryonic stem cell medium, supplemented hormonal epithelial medium, and/or a combination thereof. In some embodiments, the culture comprising the suitable coated and/or 2-dimensional substrate further comprises an embryonic stem cell medium. In some embodiments, the culture comprising the suitable coated and/or 2-dimensional substrate further comprises an embryonic stem cell medium supplemented with bFGF and/or LIF. In some embodiments, the culture comprising the suitable coated and/or 2-dimensional substrate further comprises an inhibitor of Rho-associated kinase. In some embodiments, the tissue sample comprises stroma and/or epithelium. In some embodiments, the tissue sample is placenta, umbilical cord, chorion, limbal tissue, conjunctiva, the skin, the oral mucosa, adipose tissue and/or a combination thereof. In some embodiments, the methods further comprise contacting a tissue sample comprising a plurality of multipotent cells with dispase.

Disclosed herein, in certain embodiments, are multipotent cell cultures made by the method comprising: (a) separating a plurality of multipotent cells from other bound cells and components of an extracellular matrix in a tissue sample, to form a plurality of isolated multipotent cells; (b) expanding at least one of the plurality of isolated multipotent cells in a first culture comprising a suitable 2-dimensional substrate without passing the Hayflick limit, to form a plurality of expanding multipotent cells; and (c) isolating and expanding at least one multipotent cell from the plurality of expanding multipotent cells in a second culture comprising a suitable 3-dimensional substrate, to generate a population of expanded multipotent cells (e.g., MSCs; (e.g., ASCs)). In some embodiments, the multipotent cells are mesenchymal stromal cells (MSC) and/or adipose derived stromal cells (ASC). In some embodiments, the 2-dimensional substrate mimics the extracellular environment found in tissues. In some embodiments, the 2-dimensional substrate comprises a gelatinous protein mixture secreted by Engelbreth-Holm-Swarm (EHS) mouse sarcoma cells. In some embodiments, the 2-dimensional substrate comprises as laminin, type IV collagen and heparan sulfate proteoglycans. In some embodiments, the 3-dimensional substrate mimics the extracellular environment found in tissues. In some embodiments, the 3-dimensional substrate comprises a gelatinous protein mixture secreted by Engelbreth-Holm-Swarm (EHS) mouse sarcoma cells. In some embodiments, the 3-dimensional substrate comprises as laminin, type IV collagen and heparan sulfate proteoglycans. In some embodiments, the first culture further comprises an embryonic stem cell medium, supplemented hormonal epithelial medium, and/or a combination thereof. In some embodiments, the first culture comprises an embryonic stem cell medium. In some embodiments, the embryonic stem cell medium is a human embryonic stem cell medium. In some embodiments, the embryonic stem cell medium comprises bFGF and/or LIF. In some embodiments, the second culture comprises an embryonic stem cell medium, supplemented hormonal epithelial medium, and/or a combination thereof. In some embodiments, the first culture comprises an embryonic stem cell medium. In some embodiments, the embryonic stem cell medium is a human embryonic stem cell medium. In some embodiments, the embryonic stem cell medium comprises bFGF and/or LIF. In some embodiments, the first or second culture further comprises an inhibitor of Rho-associated kinase. In some embodiments, the tissue sample comprises stroma and/or epithelium. In some embodiments, the tissue sample is placenta, umbilical cord, chorion, limbal tissue, conjunctiva, the skin, the oral mucosa, adipose tissue and/or a combination thereof. In some embodiments, the multipotent cells are separated from other bound cells and components of an extracellular matrix (but not the basement membrane) in the tissue sample by contacting the tissue sample with an enzyme that degrades interstitial components of the extracellular matrix but not basement membrane components. In some embodiments, the multipotent cells are separated from other bound cells and components of an extracellular matrix (but not the basement membrane) in the tissue sample by contacting the tissue sample with an enzyme that degrades interstitial collagen but not basement membrane collagen. In some embodiments, the multipotent cells are separated from other bound cells and components of an extracellular matrix (but not the basement membrane) in the tissue sample by contacting the tissue sample with dispase. In some embodiments, the multipotent cells are separated from other bound cells and components of an extracellular matrix (but not the basement membrane) in the tissue sample by contacting the tissue sample with a collagenase. In some embodiments, the multipotent cells are separated from other bound cells and components of an extracellular matrix (but not the basement membrane) in the tissue sample by contacting the tissue sample with collagenase A. In some embodiments, the multipotent cells are separated from other bound cells and components of an extracellular matrix (but not the basement membrane) in the tissue sample by contacting the tissue sample with dispase and collagenase A.

Disclosed herein, in certain embodiments, are methods of isolating and expanding a plurality of multipotent cells, comprising: (a) contacting a plurality of multipotent cells with a collagenase, to form a plurality of isolated multipotent cells; (b) expanding at least one of the plurality of isolated multipotent cells in a first culture comprising a suitable 2-dimensional substrate without passing the Hayflick limit, to form a plurality of expanding multipotent cells; and (c) isolating and expanding at least one multipotent cell from the plurality of expanding multipotent cells in a second culture comprising a suitable 3-dimensional substrate, to generate a population of expanded multipotent cells. In some embodiments, the multipotent cells are mesenchymal stromal cells (MSC) and/or adipose derived stromal cells (ASC). In some embodiments, the 2-dimensional substrate mimics the extracellular environment found in tissues. In some embodiments, the 2-dimensional substrate comprises a gelatinous protein mixture secreted by Engelbreth-Holm-Swarm (EHS) mouse sarcoma cells. In some embodiments, the 2-dimensional substrate comprises as laminin, type IV collagen and heparan sulfate proteoglycans. In some embodiments, the 3-dimensional substrate mimics the extracellular environment found in tissues. In some embodiments, the 3-dimensional substrate comprises a gelatinous protein mixture secreted by Engelbreth-Holm-Swarm (EHS) mouse sarcoma cells. In some embodiments, the 3-dimensional substrate comprises as laminin, type IV collagen and heparan sulfate proteoglycans. In some embodiments, the first culture further comprises an embryonic stem cell medium, supplemented hormonal epithelial medium, and/or a combination thereof. In some embodiments, the first culture comprises an embryonic stem cell medium. In some embodiments, the embryonic stem cell medium is a human embryonic stem cell medium. In some embodiments, the embryonic stem cell medium comprises bFGF and/or LIF. In some embodiments, the second culture comprises an embryonic stem cell medium, supplemented hormonal epithelial medium, and/or a combination thereof. In some embodiments, the first culture comprises an embryonic stem cell medium. In some embodiments, the embryonic stem cell medium is a human embryonic stem cell medium. In some embodiments, the embryonic stem cell medium comprises bFGF and/or LIF. In some embodiments, the first or second culture further comprises an inhibitor of Rho-associated kinase. In some embodiments, the tissue sample comprises stroma and/or epithelium. In some embodiments, the tissue sample is placenta, umbilical cord, chorion, limbal tissue, conjunctiva, the skin, the oral mucosa, adipose tissue and/or a combination thereof. In some embodiments, the methods further comprise contacting the multipotent cells with dispase.

Disclosed herein, in certain embodiments, are methods of isolating and expanding a plurality of multipotent cells, comprising: (a) contacting a plurality of multipotent cells with a collagenase and dispase, to form a plurality of isolated multipotent cells; (b) expanding at least one of the plurality of isolated multipotent cells in a first culture comprising a suitable 2-dimensional substrate without passing the Hayflick limit, to form a plurality of expanding multipotent cells; and (c) isolating and expanding at least one multipotent cell from the plurality of expanding multipotent cells in a second culture comprising a suitable 3-dimensional substrate, to generate a population of expanded multipotent cells. In some embodiments, the multipotent cells are mesenchymal stromal cells (MSC) and/or adipose derived stromal cells (ASC). In some embodiments, the 2-dimensional substrate mimics the extracellular environment found in tissues. In some embodiments, the 2-dimensional substrate comprises a gelatinous protein mixture secreted by Engelbreth-Holm-Swarm (EHS) mouse sarcoma cells. In some embodiments, the 2-dimensional substrate comprises as laminin, type IV collagen and heparan sulfate proteoglycans. In some embodiments, the 3-dimensional substrate mimics the extracellular environment found in tissues. In some embodiments, the 3-dimensional substrate comprises a gelatinous protein mixture secreted by Engelbreth-Holm-Swarm (EHS) mouse sarcoma cells. In some embodiments, the 3-dimensional substrate comprises as laminin, type IV collagen and heparan sulfate proteoglycans. In some embodiments, the first culture further comprises an embryonic stem cell medium, supplemented hormonal epithelial medium, and/or a combination thereof. In some embodiments, the first culture comprises an embryonic stem cell medium. In some embodiments, the embryonic stem cell medium is a human embryonic stem cell medium. In some embodiments, the embryonic stem cell medium comprises bFGF and/or LIF. In some embodiments, the second culture comprises an embryonic stem cell medium, supplemented hormonal epithelial medium, and/or a combination thereof. In some embodiments, the first culture comprises an embryonic stem cell medium. In some embodiments, the embryonic stem cell medium is a human embryonic stem cell medium. In some embodiments, the embryonic stem cell medium comprises bFGF and/or LIF. In some embodiments, the first or second culture further comprises an inhibitor of Rho-associated kinase. In some embodiments, the tissue sample comprises stroma and/or epithelium. In some embodiments, the tissue sample is placenta, umbilical cord, chorion, limbal tissue, conjunctiva, the skin, the oral mucosa, adipose tissue and/or a combination thereof.

Disclosed herein, in certain embodiments, are methods of expanding a plurality of multipotent cells, comprising: expanding at least one expanding multipotent cell in a culture comprising a suitable 3-dimensional substrate, to generate a population of expanded multipotent cells, wherein the 3D substrate comprises a gelatinous protein mixture secreted by Engelbreth-Holm-Swarm (EHS) mouse sarcoma cells. In some embodiments, the methods further comprise expanding at least one isolated multipotent cell in a culture comprising a suitable coated and/or 2-dimensional substrate without passing the Hayflick limit, to form a plurality of expanding multipotent cells, wherein the 2D substrate comprises a gelatinous protein mixture secreted by Engelbreth-Holm-Swarm (EHS) mouse sarcoma cells. In some embodiments, the methods further comprise separating a plurality of multipotent cells from other bound cells and components of an extracellular matrix in a tissue sample, to form a plurality of isolated multipotent cells. In some embodiments, the multipotent cells are mesenchymal stromal cells (MSC) and/or adipose derived stromal cells (ASC). In some embodiments, the 3-dimensional substrate mimics the extracellular environment found in tissues. In some embodiments, the coated and/or 2-dimensional substrate mimics the extracellular environment found in tissues. In some embodiments, the culture comprising the suitable 3-dimensional substrate further comprises an embryonic stem cell medium, supplemented hormonal epithelial medium, and/or a combination thereof. In some embodiments, the culture comprising the suitable 3-dimensional substrate further comprises an embryonic stem cell medium. In some embodiments, the culture comprising the suitable 3-dimensional substrate further comprises an embryonic stem cell medium supplemented with bFGF and/or LIF. In some embodiments, the culture comprising the suitable coated and/or 2-dimensional substrate further comprises an embryonic stem cell medium, supplemented hormonal epithelial medium, and/or a combination thereof. In some embodiments, the culture comprising the suitable coated and/or 2-dimensional substrate further comprises an embryonic stem cell medium. In some embodiments, the culture comprising the suitable coated and/or 2-dimensional substrate further comprises an embryonic stem cell medium supplemented with bFGF and/or LIF. In some embodiments, the culture comprising the suitable coated and/or 2-dimensional substrate further comprises an inhibitor of Rho-associated kinase. In some embodiments, the tissue sample comprises stroma and/or epithelium. In some embodiments, the tissue sample is placenta, umbilical cord, chorion, limbal tissue, conjunctiva, the skin, the oral mucosa, adipose tissue and/or a combination thereof. In some embodiments, separating a plurality of multipotent cells from other bound cells and components of an extracellular matrix in a tissue sample comprises contacting the tissue sample with an enzyme that degrades interstitial components of the extracellular matrix but not basement membrane components. In some embodiments, separating a plurality of multipotent cells from other bound cells and components of an extracellular matrix in a tissue sample comprises contacting the tissue sample with an enzyme that degrades interstitial collagen but not basement membrane collagen. In some embodiments, separating a plurality of multipotent cells from other bound cells and components of an extracellular matrix in a tissue sample comprises contacting the tissue sample with dispase. In some embodiments, separating a plurality of multipotent cells from other bound cells and components of an extracellular matrix in a tissue sample comprises contacting the tissue sample with a collagenase. In some embodiments, separating a plurality of multipotent cells from other bound cells and components of an extracellular matrix in a tissue sample comprises contacting the tissue sample with collagenase A. In some embodiments, separating a plurality of multipotent cells from other bound cells and components of an extracellular matrix in a tissue sample comprises contacting the tissue sample with dispase and collagenase A.

Disclosed herein, in certain embodiments, are methods of expanding a plurality of multipotent cells, comprising: expanding at least one expanding multipotent cell in a culture comprising a suitable 3-dimensional substrate, to generate a population of expanded multipotent cells, wherein the 3D substrate comprises a gelatinous protein mixture secreted by Engelbreth-Holm-Swarm (EHS) mouse sarcoma cells. In some embodiments, the methods further comprise expanding at least one isolated multipotent cell in a culture comprising a suitable coated and/or 2-dimensional substrate without passing the Hayflick limit, to form a plurality of expanding multipotent cells, wherein the 2D substrate comprises a gelatinous protein mixture secreted by Engelbreth-Holm-Swarm (EHS) mouse sarcoma cells. In some embodiments, the methods further comprise contacting a tissue sample comprising a plurality of multipotent cells with a collagenase, to form a plurality of isolated multipotent cells. In some embodiments, the multipotent cells are mesenchymal stromal cells (MSC) and/or adipose derived stromal cells (ASC). In some embodiments, the 3-dimensional substrate mimics the extracellular environment found in tissues. In some embodiments, the coated and/or 2-dimensional substrate mimics the extracellular environment found in tissues. In some embodiments, the culture comprising the suitable 3-dimensional substrate further comprises an embryonic stem cell medium, supplemented hormonal epithelial medium, and/or a combination thereof. In some embodiments, the culture comprising the suitable 3-dimensional substrate further comprises an embryonic stem cell medium. In some embodiments, the culture comprising the suitable 3-dimensional substrate further comprises an embryonic stem cell medium supplemented with bFGF and/or LIF. In some embodiments, the culture comprising the suitable coated and/or 2-dimensional substrate further comprises an embryonic stem cell medium, supplemented hormonal epithelial medium, and/or a combination thereof. In some embodiments, the culture comprising the suitable coated and/or 2-dimensional substrate further comprises an embryonic stem cell medium. In some embodiments, the culture comprising the suitable coated and/or 2-dimensional substrate further comprises an embryonic stem cell medium supplemented with bFGF and/or LIF. In some embodiments, the culture comprising the suitable coated and/or 2-dimensional substrate further comprises an inhibitor of Rho-associated kinase. In some embodiments, the tissue sample comprises stroma and/or epithelium. In some embodiments, the tissue sample is placenta, umbilical cord, chorion, limbal tissue, conjunctiva, the skin, the oral mucosa, adipose tissue and/or a combination thereof. In some embodiments, the methods further comprise contacting a tissue sample comprising a plurality of multipotent cells with dispase.

Disclosed herein, in certain embodiments, are methods of isolating and expanding a plurality of multipotent cells, comprising: (a) separating a plurality of multipotent cells from other bound cells and components of an extracellular matrix in a tissue sample, to form a plurality of isolated multipotent cells; (b) expanding at least one of the plurality of isolated multipotent cells in a first culture comprising a suitable 2-dimensional substrate without passing the Hayflick limit, to form a plurality of expanding multipotent cells; and (c) isolating and expanding at least one stem cell from the plurality of expanding multipotent cells in a second culture comprising a suitable 3-dimensional substrate, to generate a population of expanded multipotent cells. In some embodiments, the 2-dimensional substrate mimics the extracellular environment found in tissues. In some embodiments, the 2-dimensional substrate comprises a gelatinous protein mixture secreted by Engelbreth-Holm-Swarm (EHS) mouse sarcoma cells. In some embodiments, the 2-dimensional substrate comprises as laminin, type IV collagen and heparan sulfate proteoglycans. In some embodiments, the 3-dimensional substrate mimics the extracellular environment found in tissues. In some embodiments, the 3-dimensional substrate comprises a gelatinous protein mixture secreted by Engelbreth-Holm-Swarm (EHS) mouse sarcoma cells. In some embodiments, the 3-dimensional substrate comprises as laminin, type IV collagen and heparan sulfate proteoglycans. In some embodiments, the first culture further comprises an embryonic stem cell medium, supplemented hormonal epithelial medium, and/or a combination thereof. In some embodiments, the first culture further comprises an embryonic stem cell medium. In some embodiments, the first culture further comprises a human embryonic stem cell medium. In some embodiments, the second culture further comprises an embryonic stem cell medium, supplemented hormonal epithelial medium, and/or a combination thereof. In some embodiments, the second culture further comprises an embryonic stem cell medium. In some embodiments, the first culture further comprises a human embryonic stem cell medium. In some embodiments, the first or second culture further comprises an inhibitor of Rho-associated kinase. In some embodiments, the plurality of isolated multipotent cells is not separated from associated niche cells. In some embodiments, the plurality of isolated multipotent cells and their corresponding niche cells are in the form of isolated compacted cluster. In some embodiments, the tissue sample comprises stroma and/or epithelium. In some embodiments, the tissue sample is amniotic membrane derived from placenta, and/or umbilical cord. In some embodiments, the tissue sample is human amniotic membrane. In some embodiments, the tissue sample is stroma, basement membrane, and/or epithelium. In some embodiments, the tissue sample is limbal tissue, conjunctiva, the skin, the oral mucosa, and/or a combination thereof. In some embodiments, the tissue sample is human limbal tissue. In some embodiments, the multipotent cells are separated from other bound cells and components of an extracellular matrix (but not the basement membrane) in the tissue sample by contacting the tissue sample with an enzyme that degrades interstitial matrix metalloproteinase bonds but not basement matrix metalloproteinase bonds. In some embodiments, the multipotent cells are separated from other bound cells and components of an extracellular matrix (but not the basement membrane) in the tissue sample by contacting the tissue sample with an enzyme that breaks, degrades, and/or hydrolyzes interstitial elastin, collagen, gelatin, proteoglycan, fibronectin, casein, and/or combinations thereof. In some embodiments, the multipotent cells are separated from other bound cells and components of an extracellular matrix (but not the basement membrane) in the tissue sample by contacting the tissue sample with a matrix metalloproteinase, an elastase, and/or a combination thereof. In some embodiments, the multipotent cells are separated from other bound cells and components of an extracellular matrix (but not the basement membrane) in the tissue sample by contacting the tissue sample with a collagenase, a gelatinase, a stromelysin, a matrilysin, an epilysin, and/or a combination thereof. In some embodiments, the multipotent cells are separated from other bound cells and components of an extracellular matrix (but not the basement membrane) in the tissue sample by contacting the tissue sample with a collagenase. In some embodiments, the multipotent cells are separated from other bound cells and components of an extracellular matrix (but not the basement membrane) in the tissue sample by contacting the tissue sample with collagenase A, collagenase B, collagenase D, and/or a combination thereof.

Disclosed herein, in certain embodiments, are multipotent cell cultures made by the method comprising: (a) separating a plurality of multipotent cells from other bound cells and components of an extracellular matrix in a tissue sample, to form a plurality of isolated multipotent cells; (b) expanding at least one of the plurality of isolated multipotent cells in a first culture comprising a suitable 2-dimensional substrate without passing the Hayflick limit, to form a plurality of expanding multipotent cells; and (c) isolating and expanding at least one stem cell from the plurality of expanding multipotent cells in a second culture comprising a suitable 3-dimensional substrate, to generate a population of expanded multipotent cells. In some embodiments, the 2-dimensional substrate mimics the extracellular environment found in tissues. In some embodiments, the 2-dimensional substrate comprises a gelatinous protein mixture secreted by Engelbreth-Holm-Swarm (EHS) mouse sarcoma cells. In some embodiments, the 2-dimensional substrate comprises as laminin, type IV collagen and heparan sulfate proteoglycans. In some embodiments, the 3-dimensional substrate mimics the extracellular environment found in tissues. In some embodiments, the 3-dimensional substrate comprises a gelatinous protein mixture secreted by Engelbreth-Holm-Swarm (EHS) mouse sarcoma cells. In some embodiments, the 3-dimensional substrate comprises as laminin, type IV collagen and heparan sulfate proteoglycans. In some embodiments, the first culture further comprises an embryonic stem cell medium, supplemented hormonal epithelial medium, and/or a combination thereof. In some embodiments, the first culture further comprises an embryonic stem cell medium. In some embodiments, the first culture further comprises a human embryonic stem cell medium. In some embodiments, the second culture further comprises an embryonic stem cell medium, supplemented hormonal epithelial medium, and/or a combination thereof. In some embodiments, the second culture further comprises an embryonic stem cell medium. In some embodiments, the first culture further comprises a human embryonic stem cell medium. In some embodiments, the first or second culture further comprises an inhibitor of Rho-associated kinase. In some embodiments, the plurality of isolated multipotent cells are not separated from associated niche cells. In some embodiments, the plurality of isolated multipotent cells and their corresponding niche cells are in the form of isolated compacted cluster. In some embodiments, the tissue sample comprises stroma and/or epithelium. In some embodiments, the tissue sample is amniotic membrane derived from placenta, and/or umbilical cord, and/or a combination thereof. In some embodiments, the tissue sample is human amniotic membrane. In some embodiments, the tissue sample is stroma, basement membrane, and/or epithelium. In some embodiments, the tissue sample is limbal tissue, conjunctiva, the skin, the oral mucosa, and/or a combination thereof. In some embodiments, the tissue sample is human limbal tissue. In some embodiments, the multipotent cells are separated from other bound cells and components of an extracellular matrix (but not the basement membrane) in the tissue sample by contacting the tissue sample with an enzyme that degrades interstitial matrix metalloproteinase bonds but not basement matrix metalloproteinase bonds. In some embodiments, the multipotent cells are separated from other bound cells and components of an extracellular matrix (but not the basement membrane) in the tissue sample by contacting the tissue sample with an enzyme that breaks, degrades, and/or hydrolyzes interstitial elastin, collagen, gelatin, proteoglycan, fibronectin, casein, and/or combinations thereof. In some embodiments, the multipotent cells are separated from other bound cells and components of an extracellular matrix (but not the basement membrane) in the tissue sample by contacting the tissue sample with a matrix metalloproteinase, an elastase, and/or a combination thereof. In some embodiments, the multipotent cells are separated from other bound cells and components of an extracellular matrix (but not the basement membrane) in the tissue sample by contacting the tissue sample with a collagenase, a gelatinase, a stromelysin, a matrilysin, an epilysin, and/or a combination thereof. In some embodiments, the multipotent cells are separated from other bound cells and components of an extracellular matrix (but not the basement membrane) in the tissue sample by contacting the tissue sample with a collagenase. In some embodiments, the multipotent cells are separated from other bound cells and components of an extracellular matrix (but not the basement membrane) in the tissue sample by contacting the tissue sample with collagenase A, collagenase B, collagenase D, and/or a combination thereof.

Disclosed herein, in certain embodiments, are uses of a population of expanded multipotent cells obtained by the methods described herein for transplant.

Disclosed herein, in certain embodiments, are uses of a population of expanded multipotent cells obtained by the methods described herein for expanding epithelial progenitor cells and stem cells.

Disclosed herein, in certain embodiments, are uses of a population of expanded multipotent cells obtained by the methods described herein for expanding epithelial progenitor cells and stem cells in vitro. In some embodiments, the population of expanded multipotent cells obtained by the methods described herein are used to manufacture tissue grafts (e.g., bone grafts). In some embodiments, the population of expanded multipotent cells obtained by the methods described herein are used to manufacture bone grafts.

Disclosed herein, in certain embodiments, are uses of a population of expanded multipotent cells obtained by the methods described herein for expanding epithelial progenitor cells in vivo. In some embodiments, the population of expanded multipotent cells obtained by the methods described herein are used to treat a disease, disorder and/or condition characterized by progenitor cell failure (e.g., epithelial progenitor cell failure).

Disclosed herein, in certain embodiments, are uses of a population of expanded multipotent cells obtained by the methods described herein to treat a disease, disorder and/or condition characterized by a defect in bone, tendon, fat, cartilage or any combinations thereof.

### BRIEF DESCRIPTION OF THE DRAWINGS

The novel features of the invention are set forth with particularity in the appended claims. A better understanding of the features and advantages of the present invention may be obtained by reference to the following detailed description that sets forth illustrative embodiments, in which the principles of the invention are utilized, and the accompanying drawings of which:

FIG. 1: an exemplary method for phenotypic characterization of hAMSC and hAMEC.

FIGS. 2A and 2B: exemplary methods of limbal niche isolation.

FIG. 3: phenotype analysis by real time qPCR shows that niche cells expanded at the expense of losing ESC markers, when epithelial sphere growth diminished, and regained ESC Markers, when re-seeded onto thick 3-D MATRIGEL® after expansion.

FIG. 4: a tissue culture cross section demonstrating that limbal epithelial SCs might closely interact with cells in the underlying limbal stroma.

FIG. 5: niche cell isolation and purification on days D1, D3 and D6.

FIG. 6: Entire limbal epithelial SCs together with their native niche cells (NCs) can be isolated by collagenase alone.

FIG. 7. Cells Expressing Angiogenesis Markers in Human Limbal Stroma. Double immunostaining of corneo-limbo-conjunctival sections with PCK and Vim delineated the epithelium and the stroma in the limbal region (A, the white arrow indicating the border between the cornea and the limbus). In the stroma, double immunostaining of Flk-1/CD34, CD31/CD34, CD31/VWF, and α-SMA/PDGFRβ pairs showed cells expressing potential angiogenesis markers (B). Although the majority of these cells were present in the perivascular location, some were found subjacent to limbal basal epithelial cells (B, marked by white lines). Nuclei were counterstained by Hoechst 33342 (blue). Scale bar=100 μm.

FIG. 8. Isolation of Limbal Stromal Cells by Enzymatic Digestion. Dispase digestion of the limbal segment isolated an intact epithelial sheet, which exclusively contained PCK+ cells, of which few co-expressed Vim. Collagenase digestion (Coll) isolated clusters consisting of 80% PCK+ cells and 20% Vim+ cells. Following removal of the epithelial sheet by dispase, the residual stroma was digested with collagenase, resulting in D/C cell clusters floating in the medium and “residual stromal cells” (RSC) adherent on the plastic dish. D/C clusters contained 95% Vim+ cells and 5% PCK+ epithelial cells while RSC contained only Vim+ cells. Double immunostaining of Flk-1/CD34, CD31/VWF, and α-SMA/PDGFRβ pairs revealed that cells expressing angiogenesis markers were present in the above three stromal fractions. Nuclei were counterstained by Hoechst 33342 (blue). Scale bar=50 μm.

FIG. 9. Spheres of Angiogenesis Progenitors in 3D Matrigel. Single cells from collagenase-isolated (Coll) clusters, D/C clusters, and RSC were seeded in 3D Matrigel containing MESCM for 10 days. Sphere growth was noted only from Coll and D/C cells but not RSC cells (A). Compared to the expression level by cells immediately isolated (D0) set as 1, those of Flk-1, CD34, CD31, and α-SMA transcripts were significantly reduced in Coll spheres and RSC. However, those of the aforementioned transcripts and PDGFRβ transcript were significantly upregulated in D/C spheres (B, *P<0.05 and **P<0.01, n=3). Coll spheres consisted of predominately PCK+ cells while cells in D/C spheres (C) and single RSC (not shown) were all Vim+. Cells in C/D spheres uniformly expressed Flk-1, CD34, CD31, α-SMA, and PDGFRβ with low EdU nuclear labeling (C, yellow). In 5 day co-culturing experiments on 100% Matrigel, single D10 D/C cells but not D10 RSC cells stabilized the vascular network formed by HUVEC (prelabeled with red Q-tracker) (D). Nuclei were counterstained by Hoechst 33342 (blue). Scale bar=100 μm.

FIG. 10. Serial Passages on Coated Matrigel in MESCM. Single cells from Coll, D/C, or RSC were seeded at a density of 1×10⁴ per cm² and serially passaged on coated Matrigel in MESCM, resulting in spindle cells (A) with a steady growth up to P10 and a total of more than 1×10¹⁰ cells (B). In contrast, RSC cells did not grow. Compared to the expression level by cells immediately isolated (D0), spindle cells expanded from Coll and D/C exhibited a similar expression pattern up to P3, i.e., with more expression of Vim, CD73, CD90, CD105, α-SMA, and PDGFRβ transcripts (C). Scale bar=200 μm.

FIG. 11. Phenotypic Change by Serial Passage on Plastic in DF. The phenotype was determined by marker expression using RT-qPCR (A) and immunostaining (B) among D/C cells expanded on coated Matrigel (D/C) or on plastic in DF (D/C DF), and RSC cells expanded on plastic in DF (RSC DF) at P4. All three expanded cells did not express Flk-1, CD34, CD31, and CD45 transcripts. D/C cells expressed the highest level of CD73, CD90, CD105, α-SMA, and PDGFRβ transcripts and proteins, but did not express SMMHC and S100A4 transcripts and proteins (*P<0.05, and **P<0.01, n=3). Both D/C DF and RSC DF cells did not express CD105 and PDGFRβ transcripts and proteins, while the latter expressed more S100A4 transcripts and protein than the former. Nuclei were counterstained by Hoechst 33342 (blue). Scale bar=50 μm.

FIG. 12. Comparison of CFU-F among Expanded Cells. After seeding at the density of 50 cells per cm² for 12 days on plastic in DF, single cell-derived clones were stained by crystal violet. Three clones, i.e., large, small, and micro, were identified (A). CFU-F (%) in D/C cells was significantly higher than those of D/C DF cells and RSC DF cells; CFU-F (%) of D/C DF cells was significantly higher than that of RSC DF cells (B, *P<0.05 and **P<0.01, n=3). Scale bar=100 μm.

FIG. 13. Comparison of Tri-lineage Differentiation among Expanded Cells. D/C, D/C DF, and RSC DF cells at P4 were cultured in the standard adipogenesis (Adi), osteogenesis (Ost), or chondrogenesis (Chod) medium. D/C cells had a significantly higher frequency of adipocytes stained by Oil Red O (A, Adi, B), osteocytes stained based on matrix mineralization by Alizarin Red (A, Ost, C), and chondrocytes stained by Alcian Blue (A, Chon) than D/C DF and RSC DF cells (B and C, *P<0.05 and **P<0.01, n=3). Scale bar=50 μm.

FIG. 14. Comparison of Sphere Growth by Reunion between LEPC and Expanded Cells. LEPC derived from dispase-isolated limbal epithelial sheets were mixed with D/C, D/C DF, and RSC DF (all at P4), as well as BMMSC and HCF to generate sphere growth on Day 10 in 3D Matrigel containing MESCM (A). Compared to LEPC alone, expression of the ΔNp63α transcript by LEPC+D/C and LEPC+BMMSC spheres to a lesser extent was significantly upregulated, while that by LEPC+HCF cells was significantly downregulated (B, *P<0.05, **P<0.01, n=3). In contrast, expression of the CK12 transcript was significantly downregulated in LEPC+D/C but significantly upregulated in LEPC+HCF cells. The above finding of transcript expression was consistent with the protein level of p63α and CK12 based on Western blots using β-actin as a loading control (C, P<0.01) and with double immunostaining between CK12 and p63α (D).

FIG. 15. Collagenase but not dispase isolates more subjacent Vim+ cells. A tissue culture cross section demonstrating that limbal epithelial SCs might closely interact with a subset of mesenchymal cells in the underlying limbal stroma. Dispase removes the entire PCK+ epithelial sheets but Collagenase isolates both PCK+ epithelial cells and Vim+ stromal mesenchymal cells underneath the basement membrane. The isolation method can thus be removed by removing the limbal epithelial cells first before collagenase digestion, a method termed D/C method, which results in predominant Vim+ clusters floating in the digestion medium and single residual stromal cells adherent on plastic surface. The former is termed D/C cells while the latter is termed RSC cells, which are derived from the remainder of the limbal stroma including blood vessels.

FIG. 16. Collagenase-isolated Clusters Expressed More SC Markers When Digested in MESCM. qRT-PCR showed that collagenase-isolated clusters in MESCM expressed significantly more Oct4, Nanog, Sox2, Rex1, CD34 and N-Cadherin (N-Cad) transcripts than those digested in SHEM and DF (A, n=3, **P<0.01). Double immunostaining between PCK (green), p63α (red) or Vim (red) and other markers revealed that small (PCK−/p63α−/Vim+) non-epithelial cells were Oct4+(green), Nanog+(red), Sox2+(red), SSEA4+(green), Rex1+(red), CD34+(green), N-Cad+ (red) (D, arrows). Bar=20 μm.

FIG. 17. Different Growth by Collagenase-Isolated Cells in Coated, 2D and 3D Matrigel. Single cells from collagenase-isolated limbal clusters (FIG. 1) were seeded in coated, 2D, and 3D Matrigel at 5×10⁴/cm² in MESCM. Spheres emerged in 3D Matrigel while predominant spindle cells were found in coated and 2D Matrigel (A). The sphere in 3D Matrigel was formed by reunion of single PCK+ (green) cells and Vim+ (red) cells, of which both increased in cell numbers in 10 days (B). Double staining between PCK (green) and EdU (red) confirmed that EdU+ nuclei were high in coated and 2D Matrigel but low in 3D Matrigel (C). Bar=20 μm.

FIG. 18. Expansion of Spindle Cells on Coated Matrigel by Serial Passages. Single cells derived from collagenase-isolated limbal clusters were seeded at 1×10⁵/cm² on coated Matrigel in MESCM. Spindle cells emerged among small round cells in P0, rapidly proliferated, and became dominant after P2. These spindle cells in P4 could still form spheres when re-seeded in 3D Matrigel at the density of 5×10⁴/cm². Bar=100 μm.

FIG. 19. Phenotypic Characterization of Expanded Mesenchymal Cells. Compared to D0 clusters immediately isolated by collagenase, qRT-PCR revealed rapid disappearance of p63 and CK12 transcripts by P2, a significant decline of Oct4, Nanog, Sox2, and CD34 (n=3, **P<0.01), a steady increase of Vim and N-Cad (n=3, **P<0.01), and no change in Rex1 from on coated Matrigel from P0 to P3 (A). Upon being re-seeded in 3D Matrigel at P4, Oct4, Nanog, Sox2, Rex1, and CD34 transcripts were significantly increased (n=3, *P<0.05 and **P<0.01, compared to P3 cells) (A). All cells derived from P4 aggregates were Vim+ and heterogeneously expressed SC markers (B). Bar=20 μm.

FIG. 20. Unique Recovery of SC Markers by MESCM. Vim+ spindle cells were continuously expanded in MESCM, SHEM, and/or DF on coated Matrigel up to P3. Upon reseeding in 3D Matrigel on P4, qRT-PCR showed re-expression of Oct4, Nanog, Sox2, and CD34 by cells cultured in MESCM, but not in SHEM and DF (n=3, **P<0.01). When the P4 cells were harvested from 3D Matrigel in DF, immunostaining showed that all cells were Vim+ and very few were Oct4+ and Nanog+ (B, arrows). Bar=20 μm.

FIG. 21. Re-union of Dispase-isolated Epithelial Cells and Expanded MCs. In 3D Matrigel containing MESCM, dispase-isolated epithelial cells (Dispase) formed spheres (A), which consisted of PCK+ epithelial cells (B) of which few also co-expressed Vim (B, yellow, marked by stars). When single dispase-isolated epithelial cells (Dis) were mixed with P4 MCs expanded in 3D Matrigel in MESCM or DF, they also formed spheres (C and E, respectively). Such spheres consisted of epithelial cells and MCs pre-labeled by Qdot® nanocrystals (red) (D). Spheres formed by MCs isolated in DF tended to adhere to one another on Day 10 (E). Bar=100 μm in A, C and E, but 20 μm in B and D.

FIG. 22. Maintenance of Limbal Epithelial Progenitor Status by MCs Expanded in MESCM but not DF. D10 Spheres in 3D Matrigel were formed by dispase-isolated limbal epithelial cells alone (Dispase) or mixed with MCs expanded on coated Matrigel in DF (Dis+MCs (DF)) or in MESCM (Dis+MCs (MESCM)), or by collagenase-isolated clusters (Collagenase). Immunofluorescent staining of p63α demonstrated that Dispase+MCs (MESCM) had more p63α expression than Dispase+MCs (DF) (A). Western blot analysis confirmed that Dispase+MCs (MESCM) had more p63α but less CK12 than Dispase+MCs (DF) using β-actin as the loading control (B). Spheres generated by Dis+MCs (MESCM) had a significantly more holoclone than those by Dis+MCs (DF) using Dispase and Collagenase as controls (C, n=3, **P<0.01). Bar=20 μm.

FIG. 23. Serial Passages on Plastic. Cells isolated from collagenase-isolated clusters from a 62 years old nordonor were serially passaged on plastic in ESCM containing LIF and bFGF. They yielded spindle cells (A) and could only reach P4 with a doubling time of over 165 h and NCD of 6 (B). When P3 single cells were reseeded in 3D Matrigel for 6 days, they generated P4/3D aggregates at Day 6 with a smooth contour (A). Compared to D0 cells just isolated, P3 spindle cells did not express Oct4, Sox2, Flk-1, CD34, CD31, PDGFRβ, and SMMHC transcripts, but expressed α-SMA and S100A4 transcripts (C,*P<0.05, **P<0.01). Furthermore, after being reseeded in 3D Matrigel, the resultant P4/3D cells did not regain expression of the above markers. Scale bar=200 μm.

FIG. 24. Serial Passages on Coated Matrigel. Single cells derived from collagenase isolated limbal clusters from one limbal segment of the same nordonor as FIG. 1 were serially passaged on coated Matrigel in ESCM with bFGF and LIF. They generated spindle cells from P1 to P12 and had a steady proliferative rate with the doubling time of 43 to 47 h from P2 to P10 (B). Bar=100 μm.

FIG. 25. Pericyte Phenotype Promoted by Serial Passages on Coated Matrigel. When compared to freshly isolated cells at D0, RT-qPCR revealed a notable decrease of Oct4, Sox2, Flk-1, CD34, CD31, SMMHC, and S100A4 transcripts but a dramatic Page 30 increase of □-SMA and PDGFRβ transcripts during serial passages (A, n=3, *P<0.05,**P<0.01). D0 cells consisted of PCK+ and Vim+ cells and expressed Oct4 and Sox2. In addition, Vim+ cells expressed Flk-1, CD34, CD31 or α-SMA, but the overall percentage of colocalization was less than 1% (see photos with low magnification of double labeling of Flk-1/Vim, CD31/Vim and α-SMA/Vim, n=1000), and none expressed PDGFRβ (B, D0). In contrast, P3 cells were all PCK−/Vim+, α-SMA+ and PDGFRβ+, but negative to Oct4, Sox2, Flk-1, CD34, and CD31 (B,P3). Nuclei were counterstained by Hoechst 33342 (blue). Scale bar=50 μm.

FIG. 26. Angiogenesis Progenitors Promoted by Reseeding in 3D Matrigel. P3 cells expanded on coated Matrigel were reseeded in 3D Matrigel, they formed P4/3D aggregates; single cells were collected on Day 6. Compared to P3 spindle cells, expression of Oct4, Sox2, Flk-1, CD34, CD31, αSMA and PDGFRβ transcripts were markedly upregulated in P4/3D cells (A, n=3, *P<0.05, **P<0.01). In contrast, expression of SMMHC and S100A4 remained lacking. P4/3D aggregates were noted as early as 4 h and exhibited with a stellate contour at Day 6 (B). Immunostaining of P4/3D cells showed uniform expression of Vim together with the above positive markers (B). Nuclei were counterstained by Hoechst 33342 (blue). Scale bar=50 μm.

FIG. 27. Differentiation into Vascular Endothelial Cells. Single cells of P4/3D aggregates were cultured on plastic in EGM2 with VEGF for 3 days, yielding spindle cells similar to HUVEC. They uniformly expressed Flk-1, CD31, and vWF, and took up Dil-Ac-LDL (top) in a similar fashion to the positive control of HUVEC cultured in the same condition (bottom). Nuclei were counterstained by Hoechst 33342 (blue). Scale bar=100 μm.

FIG. 28. Support of HUVEC Vascular Tube Network. Fluorescence pre-labeled (red) HUVEC and P4/3D cells were seeded alone or together on the surface of 100% Matrigel in EGM2. Although vascular tube-like network was noted in all three conditions at Day 1 (A-C), such network in P4/3D cells (A) or HUVEC (B) alone was disintegrated by Day 2 (E, F). In contrast, the network formed by co-cultured P4/3D cells and HUVEC(C) was maintained at Day 2 (G) and Day 5 (not shown). High magnification of insets (C, G) revealed close association between P4/3D cells and HUVEC (red) in the network at Day 1 (D) and Day 2 (H). Scale bar=200 μm for A-C, E-G and 50 μm for D and H.

FIG. 29. Epithelial Sphere Growth in 3D Matrigel. Limbal epithelial progenitor cells (LEPC) derived from dispase-isolated epithelial sheets alone or mixed with fluorescence pre-labeled (red) HUVEC or P4/3D cells to generate sphere growth from Day 2 to Day 10 in 3D Matrigel (A). Compared to those formed by LEPC alone, spheres formed by LEPC+HUVEC and by LEPC+P4/3D expressed significantly more ΔNp63α, CK15, and CEBPδ transcripts (B, n=3, *P<0.05, **P<0.01). In addition, expression of CK12 transcripts by LEPC+HUVEC was not different from that of LEPC alone (B, n=3, Page 32P>0.05), while that by LEPC+P4/3D was not detectable (B, n=3, P<0.01). Compared to LEPC alone, expression of p63α protein was elevated in both LEPC+HUVEC and LEPC+P4/3D (C, n=3, P<0.05). In contrast, expression of CK12 protein was not reduced in LEPC+HUVEC(C, n=3, P>0.05) but was reduced to a nondetectable level in LEPC+P4/3D using β-actin as a loading control (C, n=3, P<0.05). Double staining with p63α and CK12 showed that HUVEC or P4/3D cells alone did not express p63α or CK12, while CK12 was expressed by LEPC alone and LEPC+HUVEC but notLEPC+P4/3D (D). Scale bar=200 μm for A and 100 μm for D.

FIG. 30. Exemplifies that expression of ESC and angiogenesis markers decreases if digestion with collagenase or D/C method is carried out in SHEM but not MESCM.

FIG. 31. Exemplifies that angiogenesis progenitors can be maintained and expanded better on coated Matrigel in SHEM than plastic in SHEM.

FIG. 32. Exemplification that outgrowth expansion in MESCM better preserves limbal progenitors than expansion in SHEM.

FIG. 33. Exemplification that outgrowth expansion in MESCM promotes expansion of NCs expressing ESC and angiogenesis markers.

FIG. 34. Exemplification that collagenase followed by dispase enzymatic digestion (C/D) yields higher percentage of angiogenic progenitors from hAM.

FIG. 35. Exemplification that angiogenic progenitors are better expanded on 5% MG than PL in SHEM.

FIG. 36. Exemplification that angiogenic progenitors can be expanded on 5% MG in SHEM but cannot be expanded on PL in DMEM/10% FBS.

### DETAILED DESCRIPTION OF THE INVENTION

### Certain Terminology

As used herein, “amniotic membrane” (AM), and/or amnion, means the thin, tough membrane that encloses the embryo and/or fetus. It is the innermost layer of the placenta. AM is also found in the umbilical cord. AM has multiple layers, including an epithelial layer, a basement membrane; a compact layer; a fibroblast layer; and a spongy layer.

As used herein, “basement membrane” means a thin sheet of fibers that underlies epithelium and/or endothelium. The primary function of the basement membrane is to anch and/or the epithelium and endothelium to tissue. This is achieved by cell-matrix adhesions through substrate adhesion molecules (SAMs). The basement membrane is the fusion of two lamina, the basal lamina and the lamina reticularis. The basal lamina layer is divided into two layers—the lamina lucida and the lamina densa. The lamina densa is made of reticular collagen (type IV) fibrils coated in perlecan. The lamina lucida is made up of laminin, integrins, entactins, and dystroglycans. The lamina reticularis is made of type III collagen fibers. Basement membrane is found in, amongst other locations, amniotic membrane, adipose tissue, and the corneal limbus.

As used herein, the term “stem cell niche” means the microenvironment in which stem cells are found. The stem cell niche regulates stem cell fate. It generally maintains stem cells in a quiescent state to avoid their depletion. However, signals from stem cell niches also signal stem cells to differentiate. Control over stem cell fate results from, amongst other factors, cell-cell interactions, adhesion molecules, extracellular matrix components, oxygen tension, growth factors, cytokines, and the physiochemical nature of the niche.

The terms “subject” and “individual” are used interchangeably. As used herein, both terms mean any animal, preferably a mammal, including a human and/or non-human. The terms patient, subject, and individual are used interchangeably. None of the terms are to be interpreted as requiring the supervision of a medical professional (e.g., a doctor, nurse, physician's assistant, orderly, hospice worker).

The terms “treat,” “treating” or “treatment,” as used herein, include alleviating, abating and/or ameliorating a disease and/or condition symptoms, preventing additional symptoms, ameliorating and/or preventing the underlying metabolic causes of symptoms, inhibiting the disease and/or condition, e.g., arresting the development of the disease and/or condition, relieving the disease and/or condition, causing regression of the disease and/or condition, relieving a condition caused by the disease and/or condition, and/or stopping the symptoms of the disease and/or condition either prophylactically or therapeutically.

### Multipotent Stromal Cells

Multipotent Stromal Cells (MSCs), or alternatively mesenchymal stem cells, are multipotent cells that have the ability to differentiate into a variety of cell types, including: osteoblasts, chondrocytes, adipocytes, pericytes. MSCs have a large capacity for self-renewal while maintaining their multipotency.

MSCs have been isolated from placenta, umbilical cord tissue, namely Wharton's jelly and the umbilical cord blood, amniotic membrane (AM), amniotic fluid, adipose tissue, the corneal limbus, bone marrow, peripheral blood, liver, skin, and the corneal limbus. Currently, efforts to isolate MSCs focus on the perivascular space and the pericytes; however, the inventors of this application have discovered that basement membranes are an alternative source of MSCs. For example, in the limbus the best source of MSCs is not the perivascular area but the basement membrane subadjacent to the limbal epithelium. MSCs have also been isolated from the avascular stroma of the amniotic membrane.

Human AM contains two different cell types derived from two different embryological origins: amniotic membrane epithelial cells (hAMEC) are derived from the embryonic ectoderm, while human amniotic membrane stromal cells (hAMSC) are derived from the embryonic mesoderm and are sparsely distributed in the stroma underlying the amnion epithelium. Phenotypically, hAMEC uniformly express epithelial markers, for example CK 8, CK14, CK17, CK18, CK19, SSEA3, SSEA4, Tra-1-60, Tra-1-81, Oct4, nanog, and sox2. hAMECs also express the mesenchymal marker vimentin (Vim) in some scattered clusters. hAMSCs express the mesenchymal cell marker vimentin (Vim) but not pancytokeratins (PCK), α-smooth muscle actin (α-SMA) and/or desmin. MSCs also express Oct4, Sox2, Nanog, Rex1, SSEA4, nestin, N-cadherin, and CD34. Little is known whether the avascular property of AM contain angiogenic expressing cells in hAMEC and/or hAMSC in vivo and whether the AM expressing ESC markers might represent a subset that might be different from those not expressing ESC markers and angiogenic markers, and if so, whether they can be separately isolated. It also remains unclear whether these markers were also expressed in AM stroma. MSCs have been expanded from both hAMEC and hAMSC.

Multipotent stromal cells are long, thin cells with a small cell body. The cells have a round nucleus with a prominent nucleolus. The nucleus is surrounded by finely dispersed chromatin particles. The cells also have a small amount of Golgi apparatus, rough endoplasmic reticulum, mitochondria, and polyribosomes.

There is currently no test that can be performed on a single cell to determine whether that cell is an MSC. There are surface antigens that can be used to isolate a population of cells that have similar self-renewal and differentiation capacities, yet MSCs, as a population, typically do not all express the proposed markers; and it is not certain which ones must be expressed in order for that cell to be classified as an MSC.

Current isolation and culturing techniques are crude and result in low yields of MSCs (see, Tables 1-5). For example, hAMECs have been isolated from the AM stroma by use of trypsin/EDTA (T/E) and/or dispase (D), and collagenase digestion has been used later to release hAMSC. However, protocols have not clearly defined nor documented whether MSCs are derived from hAECs or hAMSCs during isolation or both. Further, these methods result in high yield of hAMEC (<2% vim+ cells) and low epithelial contamination of hAMSC (<1% of cytokeratin+). Current isolation and expansion methods for MSCs are carried out in a basal nutrient medium supplemented with fetal bovine serum. There is a need for new methods of preferentially isolating and expanding MSCs.

TABLE 1

| | | 2. {Miki, 2005 | | |
|---|---|---|---|---|
| | | 10276 /id} | | |
| | 1 {Yen, 2005 | {Miki, 2007 14170 | 3. {Portmann-Lanz, | 4. {Sudo, 2007 |
| | 14105 /id} | /id} | 2006 11426 /id} | 11429 /id} |
| Tissue Source | hAMEC | hAMEC | hAMSC, chorion | mainly hAMSC |
| | | | vs hBM-MSC | |
| Isolation Method | 0.25% trypsin- | 2^{nd} and 3^{rd} 0.05% | Dispase/1 h, scraping, | Mince follow |
| | EDTA (P) | Trypsin/EDTA.30 mins | treated with T/E, | Collagenase, |
| | | | Collagenase II on | then T/E, |
| | | | HAMSC | filtration |
| Substrate | PL | PL | PL | PL |
| Culture Medium | DMEM, | DMEM, | a-MEM, | a-MEM, |
| NEAA: non- | 10% FBS | 10% FBS, | 20% FBS. | 10% FBS |
| essential amino- | | NEAA, 2-Me, L- | | |
| acid; 2-Me: 2- | | glu, EGF | | |
| mercaptoethanol, | | | | |
| L-glu: L- | | | | |
| glutamine | | | | |
| Passage | Early Passage | 6 | test up to 4-5 | ND up to 3 |

TABLE 2

| | | | 7. |
|---|---|---|---|
| | | 6. {Alviano, | {Ilancheran, |
| | 5. {Soncini, 2007 | 2007 | 2007 |
| | 11431 /id} | 11428/id} | 14098 /id} |
| Tissue Source | hAMSC, hCSC | mainly hAMSC | hAMEC |
| | BM | | |
| Isolation Method | Dispase (7 min) | *Mince -> T/E | 0.25% T/Ex1 |
| | collagenase(3 h) | * collagenase | |
| | Filtration | IV+ DNAse I | |
| | Select CD271+ | | |
| | cells | | |
| Substrate | PL | PL | PL |
| Culture Medium: | RPMI-1640, | DMEM, | DMEM/F12, |
| NEAA: non-essential | 10% FCS | 20% FBS | 10% FBS |
| amino-acid; 2-Me: 2- | | | |
| mercaptoethanol, | | | |
| L-glu: | | | |
| L-glutamine | | | |
| Passage | Colonies passage | 15 | ND |
| | for p15 | | |

TABLE 3

| | 8. {Wolbank, | 9. {Bilic, 2008 | 10. {Kim, 2007 |
|---|---|---|---|
| | 2007 11430 /id} | 14171 /id} | 14173 /id} |
| Tissue Source | hAMEC, | hAMEC, hAMSC | hAMSC |
| | hAMSC, BM, | | |
| | Adipocyte | | |
| Isolation Method | 0.25% T/E x3 | *T/E, Dispase, | *T/Ex2 |
| | Then collagenase I | scraping,, | *collagenase + DNase |
| | | *Collagenase A | |
| Substrate | PL | PL | PL |
| Culture Medium: | DMEM/F12, | DMEM/F12, | low glu DMEM, |
| NEAA: non-essential | 10% FBS | 10% FBS | 10% FFBS |
| amino-acid; 2-Me: 2- | | | |
| mercaptoethanol, L-glu: L- | | | |
| glutamine | | | |
| Passage | 6 | 5 | 15 |

TABLE 4

| | | | | {Battula, 2007 |
|---|---|---|---|---|
| | Yang, 2009 | Yu, 2006 | Dravida, 2005 | 14115 /id} |
| Tissue | H dermal | Hair Follical | H Cornea | Placenta |
| Source | Scar tissue | | Limbus | (chorion and |
| | | | | AM) |
| Isolation | Cut then | Dispase, | -Dispase remove | Collagenase + Dispase |
| Method | 0.02% T/E | T/E | epi | |
| | | | -Explant stromal | |
| | | | tissue | |
| Substrate | bacteriological | PL | 1% Matrigel | 0.1% gelatin coat |
| | culture | | | |
| | dishes | | | |
| Culture | DMEM, | DMEM, | DMEM/F12 | DMEM/ |
| Medium | 20% FBS, | 20% KO, | (1:1) | 20% KO serum, |
| GFs | NEAA; 2- | NEAA, 2- | 10% KO, 2-Me | NEAA, 2-Me, L- |
| | Me, | Me, | bFGF(4 ng/ml) | glu, bFGF |
| | BMP4(30 ng/ml) | bFGF | LIF (10 ng/ml) | |
| | | | ITS | |
| Further | N/A | N/A | SSEA4+ cells | N/A |
| Isolation | | | | |
| Embryonic | Oct 4; Sox2, | Low Oct 4, | Oct 4; Nanog, | Oct 4, FZD9 |
| markers | Nestin, c- | nanog | Rex, TDGF-1, | |
| | kit, AP, | | Sox2 | |
| | SSEA4, | | | |
| Other | | | CD31+* | |
| Markers | | | | |
| MSC | CD90 | | CD73, CD105 | CD73, MHCI, |
| Marker | | | | MHCII |
| | | | | CD90, CD105, |
| | | | | CD49d (high in |
| | | | | hAMSC) |
| Negative | | SSEA-3, | CD34, CD43, | CD34 and CD45 |
| markers | | SSEA-4 | CD123, CD14, | CD49d (low in |
| | | | CD106, HLA- | hAMEC) |
| | | | DR | |
| Approach/ | Neuronal | meso | Ecto, meso, endo | Pancreatic, |
| Differentiation | potential | | | neuron- and |
| | | | | astrocyte- |
| | | | | markers, Adipo, |
| | | | | osteo, |
| Up to | 14 | N/A | 20 | |
| Passage | | | | |

TABLE 5
Ex Vivo Expansion Protocols of Autologous Human Limbal Epithelial Cells

| | Protocols | | | | | |
|---|---|---|---|---|---|---|
| | | | | 4 | | 6 |
| | | | 3 | Nakamura | 5 | Shimazaki |
| | 1 | 2 | Tsai | 2004{Nakamura, | Sangwan | 2002{Shimazaki, |
| | Pellegrini | Schwab | 2000{Tsai, | 2004 | 2003{Sangwan, | 2002 3653 /id} |
| | 1997{Pellegrini, | 2000{Schwab, | 2000 | 5103 /id} | 2003 | and Kawashima |
| | 1997 810 | 2000 | 2298 | and 2006{Nakamura, | 4414 | 2007{Kawashima, |
| | /id} | 2303 /id} | /id} | 2006 11422 /id} | /id} | 2007 11439 /id} |
| Epithelial | Trypsin/ | Trypsion/ | Brief | Cut into | Cut into | Cut into |
| Isolation from | EDTA | EDTA | Dispase | small pieces | Small pieces | Small pieces |
| Limbal Biopsy | | | | and/or | | |
| | | | | Dispase and | | |
| | | | | Trypsin/ | | |
| | | | | EDTA | | |
| 3T3 Coculturing | Yes | Yes | No | No | No | No |
| for Pre- | | | | | | |
| amplification | | | | | | |
| Co-culturing with | No | No | No | Yes | No | Yes with air- |
| 3T3 on Plastic | | | | | | lifting |
| Substrate | Fibrin | dAM | iAM | dAM | dAM | dAM |
| | | by Trypsin/ | | by EDTA | by Trypsin/ | by NH₄OH and |
| | | EDTA | | and scraping | EDTA and | scraping |
| | | | | | scraping | |
| Air-lifting | No | No | No | Yes | No | Yes |
| Days required to | NA, NA | NA | 14-21 | at least 14 | 14-21 | 14 |
| reach a | | | | | | |
| transplantable | | | | | | |
| size | | | | | | |

[Note]
Abbreviation used:
CTX: Cholera Toxin;
D: DMEM;
D/F: DMEM/F12;
dAM: denuded AM;
iAM: intact AM;
K: KGM;
M: MEM

### Isolation of Multipotent Cells

The present application provides a new method of isolating and expanding a plurality of multipotent cells. In some embodiments, the methods comprise (a) separating a plurality of multipotent cells from other bound cells and components of an extracellular matrix in a tissue sample, to form a plurality of isolated multipotent cells; (b) expanding at least one of the plurality of isolated multipotent cells in a first culture comprising a suitable 2-dimensional substrate without passing the Hayflick limit to form a plurality of expanding multipotent cells; and (c) isolating and expanding at least one expanding multipotent cell from the plurality of expanding multipotent cells in a second culture comprising a suitable 3-dimensional substrate, to generate a population of expanded multipotent cells.

In some embodiments, the methods comprise (a) separating a plurality of multipotent cells from other bound cells and components of an interstitial extracellular matrix in a tissue sample, to form a plurality of isolated multipotent cells, wherein the plurality of multipotent cells are not separated from basement membrane; (b) expanding at least one of the plurality of isolated multipotent cells in a first culture comprising a suitable 2-dimensional substrate without passing the Hayflick limit to form a plurality of expanding multipotent cells; and (c) isolating and expanding at least one expanding multipotent cell from the plurality of expanding multipotent cells in a second culture comprising a suitable 3-dimensional substrate, to generate a population of expanded multipotent cells.

In some embodiments, the methods comprise (a) contacting a tissue sample with a collagenase to separate a plurality of multipotent cells from other bound cells and components of an extracellular matrix in a tissue sample, to form a plurality of isolated multipotent cells; (b) expanding at least one of the plurality of isolated multipotent cells in a first culture comprising a suitable 2-dimensional substrate without passing the Hayflick limit to form a plurality of expanding multipotent cells; and (c) isolating and expanding at least one expanding multipotent cell from the plurality of expanding multipotent cells in a second culture comprising a suitable 3-dimensional substrate, to generate a population of expanded multipotent cells.

In some embodiments, the methods comprise (a) contacting a tissue sample with dispase and a collagenase to separate a plurality of multipotent cells from other bound cells and components of an extracellular matrix in a tissue sample, to form a plurality of isolated multipotent cells; (b) expanding at least one of the plurality of isolated multipotent cells in a first culture comprising a suitable 2-dimensional substrate without passing the Hayflick limit to form a plurality of expanding multipotent cells; and (c) isolating and expanding at least one expanding multipotent cell from the plurality of expanding multipotent cells in a second culture comprising a suitable 3-dimensional substrate, to generate a population of expanded multipotent cells.

In some embodiments, the methods comprise (a) contacting a tissue sample with a collagenase to separate a plurality of multipotent cells from other bound cells and components of an extracellular matrix in a tissue sample, to form a plurality of isolated multipotent cells, wherein the collagenase degrades interstitial collagen but not basement membrane collagen; (b) expanding at least one of the plurality of isolated multipotent cells in a first culture comprising a suitable 2-dimensional substrate without passing the Hayflick limit to form a plurality of expanding multipotent cells; and (c) isolating and expanding at least one expanding multipotent cell from the plurality of expanding multipotent cells in a second culture comprising a suitable 3-dimensional substrate, to generate a population of expanded multipotent cells.

In some embodiments, the methods comprise (a) contacting a tissue sample with dispase and a collagenase to separate a plurality of multipotent cells from other bound cells and components of an extracellular matrix in a tissue sample, to form a plurality of isolated multipotent cells, wherein the dispase and collagenase degrade interstitial components of the extracellular membrane but not basement membrane components; (b) expanding at least one of the plurality of isolated multipotent cells in a first culture comprising a suitable 2-dimensional substrate without passing the Hayflick limit to form a plurality of expanding multipotent cells; and (c) isolating and expanding at least one expanding multipotent cell from the plurality of expanding multipotent cells in a second culture comprising a suitable 3-dimensional substrate, to generate a population of expanded multipotent cells.

Disclosed herein, in certain embodiments, are methods of expanding a plurality of multipotent cells, comprising: expanding at least one isolated multipotent cell in a culture comprising a suitable coated and/or 2-dimensional substrate without passing the Hayflick limit, to form a plurality of expanding multipotent cells, wherein the 2D substrate comprises a gelatinous protein mixture secreted by Engelbreth-Holm-Swarm (EHS) mouse sarcoma cells. In some embodiments, the methods further comprise expanding at least one expanding multipotent cell in a culture comprising a suitable 3-dimensional substrate, to generate a population of expanded multipotent cells, wherein the 3D substrate comprises a gelatinous protein mixture secreted by Engelbreth-Holm-Swarm (EHS) mouse sarcoma cells. In some embodiments, the methods contacting a tissue sample comprising a plurality of multipotent cells with a collagenase, to form a plurality of isolated multipotent cells.

Disclosed herein, in certain embodiments, are methods of expanding a plurality of multipotent cells, comprising: expanding at least one expanding multipotent cell in a culture comprising a suitable 3-dimensional substrate, to generate a population of expanded multipotent cells, wherein the 3D substrate comprises a gelatinous protein mixture secreted by Engelbreth-Holm-Swarm (EHS) mouse sarcoma cells. In some embodiments, the methods further comprise expanding at least one isolated multipotent cell in a culture comprising a suitable coated and/or 2-dimensional substrate without passing the Hayflick limit, to form a plurality of expanding multipotent cells, wherein the 2D substrate comprises a gelatinous protein mixture secreted by Engelbreth-Holm-Swarm (EHS) mouse sarcoma cells. In some embodiments, the methods further comprise contacting a tissue sample comprising a plurality of multipotent cells with a collagenase, to form a plurality of isolated multipotent cells.

The above-described method (a first expansion on Matrigel coated substrate and/or 2-dimensional Matrigel, followed by a second expansion in 3-dimensional Matrigel) enables optimal expansion of MSC cells. The inventors discovered that isolated MSC cells will not proliferate in 3D Matrigel but that they will proliferate on a substrate coated in Matrigel and/or in 2D Matrigel. However, expansion on a substrate coated in Matrigel and/or in 2D Matrigel results in (transient) loss of ESC and angiogenesis markers. Expression of ESC and angiogenesis markers is recovered when the MSC cells are cultured in 3D Matrigel. When cultured on plastic, as according to the conventional methods, the ESC phenotype is irreversibly lost. Additionally, the inventors discovered that the first expansion and the second expansion preferably occurs in MESCM (ESCM supplemented with bFGF and LIF) and/or the ESC phenotype is irreversibly lost.

In some embodiments, the multipotent cells are mesenchymal stromal cells (MSCs). In some embodiments, the MSCs are found in contact with a basement membrane. In some embodiments, the MSCs are found in the corneal limbus. In some embodiments, the MSCs are found in the amniotic membrane, for example in the avascular stroma. In some embodiments, the MSCs are adipose stromal cells (ASC).

The first culture of a method described herein may, in certain instances, further comprise an embryonic stem cell medium, supplemented hormonal epithelial medium, and/or a combination thereof. In some embodiments, the first culture further comprises an embryonic stem cell medium, which may be a human embryonic stem cell medium. In some embodiments, the first culture may further comprise an inhibitor of Rho-associated kinase. Kinase activity is inhibited by the intramolecular binding between the C-terminal cluster of RBD domain and the PH domain to the N-terminal kinase domain of ROCK. Thus, the kinase activity is off when ROCK is intramoleculary folded.

The second culture of a method described herein may, in certain instances, further comprise an embryonic stem cell medium, supplemented hormonal epithelial medium, a medium containing high levels of calcium and serum, and/or a combination thereof. In some embodiments, the second culture further comprises an embryonic stem cell medium, which may be a human embryonic stem cell medium.

### Isolation

In some embodiments, multipotent cells (e.g., MSCs) are isolated from other bound cells (e.g., epithelial cells) and components of an extracellular matrix (e.g., stromal extracellular matrix) in a tissue sample by contacting the tissue sample with a protease. In some embodiments, the multipotent cells (e.g., MSCs) are isolated from other bound cells and components of an extracellular matrix (e.g., stromal extracellular matrix) in the tissue sample by contacting the tissue sample with a protease that degrades and/or hydrolyzes components of the interstitial space (e.g., stroma) but not components of the basement membrane (e.g., collagens, heparan sulfate proteoglycans, laminin, and nidogen). In some embodiments, the multipotent cells (MSCs) are isolated from other bound cells and components of an extracellular matrix (e.g., stromal extracellular matrix) in the tissue sample by contacting the tissue sample with dispase. Dispase cleaves fibronectin, collagen IV, and collagen I. In some embodiments, the multipotent cells (e.g., MSCs) are isolated from other bound cells (e.g., epithelial cells) and components of an extracellular matrix (e.g., stromal extracellular matrix) in a limbal tissue sample by contacting the tissue sample with a protease (e.g., dispase) before being contacted with a collagenase.

In some embodiments, the multipotent cells (MSCs) are isolated from other bound cells (e.g., epithelial cells) and components of an extracellular matrix (e.g., stromal extracellular matrix) in a tissue sample by contacting the tissue sample with an enzyme that hydrolyzes and/or degrades interstitial (e.g., stromal) collagen but not basement membrane collagen. In some embodiments, the multipotent cells (e.g., MSCs) are separated from other bound cells (e.g., epithelial cells) and components of an extracellular matrix (e.g., stromal extracellular matrix) in a tissue sample by contacting the tissue sample with a collagenase. In some embodiments, the multipotent cells (e.g., MSCs) are separated from other bound cells (e.g., epithelial cells) and components of an extracellular matrix (e.g., stromal extracellular matrix) in a tissue sample by contacting the tissue sample with collagenase A, collagenase B, collagenase D, and/or a combination thereof. In some embodiments, the multipotent cells (e.g., MSCs) are separated from other bound cells (e.g., epithelial cells) and components of an extracellular matrix (e.g., stromal extracellular matrix) in a tissue sample by contacting the tissue sample with collagenase A.

In some embodiments, the multipotent cells (MSCs) are isolated from other bound cells (e.g., epithelial cells) and components of an extracellular matrix in the tissue sample by contacting the tissue sample with dispase and a collagenase. In some embodiments, the multipotent cells (MSCs) are isolated from other bound cells (e.g., epithelial cells) and components of an extracellular matrix in the tissue sample by contacting the tissue sample with dispase and collagenase A.

In some embodiments, the multipotent cells (e.g., MSCs) are isolated from other bound cells (e.g., epithelial cells) and components of an extracellular matrix (e.g., stromal extracellular matrix) in a limbal tissue sample by contacting the limbal tissue sample with a protease (e.g., dispase) before being contacted with a collagenase. In some embodiments, the multipotent cells (e.g., MSCs) are isolated from other bound cells (e.g., epithelial cells) and components of an extracellular matrix (e.g., stromal extracellular matrix) in an amniotic membrane or adipose tissue sample by contacting the amniotic membrane or adipose tissue sample with a collagenase before being contacted with a protease (e.g., dispase). In some embodiments, the multipotent cells (e.g., MSCs) are isolated from other bound cells (e.g., epithelial cells) and components of an extracellular matrix (e.g., stromal extracellular matrix) in an amniotic membrane or adipose tissue sample by contacting the amniotic membrane or adipose tissue sample with a collagenase, and not with dispase.

### Expansion

In some embodiments, isolated multipotent cells (e.g., MSCs) are subjected to a first expansion. In some embodiments, the first expansion occurs on a coated and/or 2-dimensional substrate. In some embodiments, the substrate is coated in composition that mimics the basement membrane and/or comprises components of the basement membrane, such as such as laminin, type IV collagen and heparan sulfate proteoglycan. In some embodiments, the substrate is coated in a gelatinous protein mixture secreted by Engelbreth-Holm-Swarm (EHS) mouse sarcoma cells. In some embodiments, the substrate is coated in Matrigel. In some embodiments, the 2-dimensional substrate mimics the basement membrane and/or comprises components of the basement membrane, such as such as laminin, type IV collagen and heparan sulfate proteoglycan. In some embodiments, the 2-dimensional substrate is a gelatinous protein mixture secreted by Engelbreth-Holm-Swarm (EHS) mouse sarcoma cells. In some embodiments, the 2-dimensional substrate is Matrigel. In some embodiments, expansion on a coated and/or 2-dimensional substrate (e.g., a Matrigel coated and/or 2D substrate) results in proliferation of multipotent cells (e.g., MSCs). In some embodiments, expansion on a coated and/or 2-dimensional substrate (e.g., a Matrigel coated and/or 2D substrate) results in proliferation of multipotent cells (e.g., MSCs) and transient loss of expression of embryonic stem cell (ESC) markers.

In some embodiments, isolated multipotent cells (e.g., MSCs) are subjected to a second expansion after the first expansion. In some embodiments, the second expansion occurs on a 3-dimensional substrate. In some embodiments, the 3-dimensional substrate mimics the basement membrane and/or comprises components of the basement membrane, such as such as laminin, type IV collagen and heparan sulfate proteoglycan. In some embodiments, the 3-dimensional substrate is a gelatinous protein mixture secreted by Engelbreth-Holm-Swarm (EHS) mouse sarcoma cells. In some embodiments, the 3-dimensional substrate is Matrigel. In some embodiments, expansion on a 3-dimensional substrate (e.g., a Matrigel 3D substrate) results in the cells from the first expansion regaining expression of ESC markers. In some embodiments, expansion of MSCs on a 3-dimensional substrate (e.g., a Matrigel 3D substrate) in the presence epithelial cells of results in the formation of epithelial/MSC spheres/aggregates.

### Medium

In some embodiments, isolation of the multipotent cells takes place in embryonic stem cell medium, supplemented hormonal epithelial medium, and/or a combination thereof. In some embodiments, isolation of the multipotent cells takes place in embryonic stem cell medium. In some embodiments, isolation of the multipotent cells takes place in human embryonic stem cell medium. In some embodiments, isolation of the multipotent cells takes place in human embryonic stem cell medium supplemented with bFGF and LIF.

In some embodiments, the first expansion takes place in culture comprising embryonic stem cell medium, supplemented hormonal epithelial medium, and/or a combination thereof. In some embodiments, the first expansion takes place in culture comprising embryonic stem cell medium. In some embodiments, the first expansion takes place in culture comprising human embryonic stem cell medium. In some embodiments, the first expansion takes place in culture comprising human embryonic stem cell medium supplemented with bFGF and LIF. In some embodiments, the first expansion takes place in culture further comprising an inhibitor of Rho-associated kinase (ROCK inhibitor). In some embodiments, use of DMEM medium (containing 10% FBS) for the first culture results in irreversible loss of ESC markers.

In some embodiments, the second expansion takes place in culture comprising embryonic stem cell medium, supplemented hormonal epithelial medium, and/or a combination thereof. In some embodiments, the second expansion takes place in culture comprising embryonic stem cell medium. In some embodiments, the second expansion takes place in culture comprising human embryonic stem cell medium. In some embodiments, the second expansion takes place in culture comprising human embryonic stem cell medium supplemented with bFGF and LIF. In some embodiments, the second expansion takes place in culture further comprising an inhibitor of Rho-associated kinase (ROCK inhibitor).

### Rock Inhibitors

The physical property of 3D MATRIGEL® differs from that of 2D in matrix rigidity. Matrix stiffness and/or rigidity has shown to direct link to cell shape change and regulate commitment lineage specific markers and differentiation in hMSCs. When cell spreading on 2D environment, one of key regulator, small GAPase RhoA, modulate the actin cytoskeleton organization, cell adhesion and migration, gene expression, microtubule dynamics, and vesicle transport and has critical role in cell cycle progression through G₁ phase.

The downstream rho effector, Rho-associated kinase (ROCK), play a central role in inducing the formation of actin-related structures such as focal adhesions and stress fibers and phosphorylates myosin light chain to induce actomyosin contractility. Inhibition of Rock activities has demonstrated to promote adhesion and proliferation in hESC, in human Wharton's jelly stem cells and in mouse osteoblast cells. Intriguingly, during inhibitor treatment, a distinct cell-cell contact disintegration without affecting its ES markers expression with, and/or without, coating MATRIGEL® and such cell contact can be reversible suggesting inhibition rock activities may maintain SC sternness. Furthermore, Rock inhibitors also have anti-apoptotic effect in enhancing the survival rate and cloning efficiency of hESC upon freeze and thaw. Although the critical role of Rho-Rock signaling has been implicated in early embryogenesis and in many other ESC in vitro model, the role of Rock inhibitor in SCs isolated from amniotic tissues remains mostly unknown.

The present inventors have identified for the first time that a Rock inhibitor can be used to promote and/or maintain the sternness of SCs if there is a concern of losing the original in vivo ESC phenotype and limited cell passage during the above expansion of hAMEC and hAMSC in 2D MATRIGEL®.

### Mesenchymal Stromal Cells (MSCs)

Disclosed herein, in certain embodiments, are methods of isolating and expanding mesenchymal stromal cells. In some embodiments, the methods comprise (a) separating a plurality of MSCs from other bound cells and components of an extracellular matrix in a tissue sample, to form a plurality of isolated multipotent cells; (b) expanding at least one of the plurality of isolated MSCs in a first culture comprising a suitable 2-dimensional substrate without passing the Hayflick limit to form a plurality of expanding MSCs; and (c) isolating and expanding at least one expanding MSC from the plurality of expanding MSCs in a second culture comprising a suitable 3-dimensional substrate, to generate a population of expanded MSCs.

In some embodiments, the methods comprise (a) separating a plurality of MSCs from other bound cells and components of an interstitial extracellular matrix in a tissue sample, to form a plurality of isolated MSCs, wherein the plurality of MSCs are not separated from basement membrane; (b) expanding at least one of the plurality of isolated MSCs in a first culture comprising a suitable 2-dimensional substrate without passing the Hayflick limit to form a plurality of expanding MSCs; and (c) isolating and expanding at least one expanding multipotent cell from the plurality of expanding MSCs in a second culture comprising a suitable 3-dimensional substrate, to generate a population of expanded MSCs.

In some embodiments, the methods comprise (a) contacting a tissue sample with a collagenase to separate a plurality of MSCs from other bound cells and components of an extracellular matrix in a tissue sample, to form a plurality of isolated MSCs; (b) expanding at least one of the plurality of isolated MSCs in a first culture comprising a suitable 2-dimensional substrate without passing the Hayflick limit to form a plurality of expanding MSCs; and (c) isolating and expanding at least one expanding multipotent cell from the plurality of expanding MSCs in a second culture comprising a suitable 3-dimensional substrate, to generate a population of expanded MSCs.

In some embodiments, the methods comprise (a) contacting a tissue sample with dispase and a collagenase to separate a plurality of MSCs from other bound cells and components of an extracellular matrix in a tissue sample, to form a plurality of isolated MSCs; (b) expanding at least one of the plurality of isolated MSCs in a first culture comprising a suitable 2-dimensional substrate without passing the Hayflick limit to form a plurality of expanding MSCs; and (c) isolating and expanding at least one expanding multipotent cell from the plurality of expanding MSCs in a second culture comprising a suitable 3-dimensional substrate, to generate a population of expanded MSCs.

In some embodiments, the methods comprise (a) contacting a tissue sample with a collagenase to separate a plurality of MSCs from other bound cells and components of an extracellular matrix in a tissue sample, to form a plurality of isolated MSCs, wherein the collagenase degrades interstitial collagen but not basement membrane collagen; (b) expanding at least one of the plurality of isolated MSCs in a first culture comprising a suitable 2-dimensional substrate without passing the Hayflick limit to form a plurality of expanding MSCs; and (c) isolating and expanding at least one expanding multipotent cell from the plurality of expanding MSCs in a second culture comprising a suitable 3-dimensional substrate, to generate a population of expanded MSCs.

In some embodiments, the methods comprise (a) contacting a tissue sample with dispase and a collagenase to separate a plurality of MSCs from other bound cells and components of an extracellular matrix in a tissue sample, to form a plurality of isolated MSCs, wherein the dispase and collagenase degrade interstitial components of the extracellular membrane but not basement membrane components; (b) expanding at least one of the plurality of isolated MSCs in a first culture comprising a suitable 2-dimensional substrate without passing the Hayflick limit to form a plurality of expanding MSCs; and (c) isolating and expanding at least one expanding multipotent cell from the plurality of expanding MSCs in a second culture comprising a suitable 3-dimensional substrate, to generate a population of expanded MSCs.

In some embodiments, the methods comprise expanding a plurality of MSCs, comprising: expanding at least one isolated multipotent cell in a culture comprising a suitable coated and/or 2-dimensional substrate without passing the Hayflick limit, to form a plurality of expanding MSCs, wherein the 2D substrate comprises a gelatinous protein mixture secreted by Engelbreth-Holm-Swarm (EHS) mouse sarcoma cells. In some embodiments, the methods further comprise expanding at least one expanding multipotent cell in a culture comprising a suitable 3-dimensional substrate, to generate a population of expanded MSCs, wherein the 3D substrate comprises a gelatinous protein mixture secreted by Engelbreth-Holm-Swarm (EHS) mouse sarcoma cells. In some embodiments, the methods contacting a tissue sample comprising a plurality of MSCs with a collagenase, to form a plurality of isolated MSCs.

In some embodiments, the methods comprise expanding a plurality of MSCs, comprising: expanding at least one expanding multipotent cell in a culture comprising a suitable 3-dimensional substrate, to generate a population of expanded MSCs, wherein the 3D substrate comprises a gelatinous protein mixture secreted by Engelbreth-Holm-Swarm (EHS) mouse sarcoma cells. In some embodiments, the methods further comprise expanding at least one isolated multipotent cell in a culture comprising a suitable coated and/or 2-dimensional substrate without passing the Hayflick limit, to form a plurality of expanding MSCs, wherein the 2D substrate comprises a gelatinous protein mixture secreted by Engelbreth-Holm-Swarm (EHS) mouse sarcoma cells. In some embodiments, the methods further comprise contacting a tissue sample comprising a plurality of MSCs with a collagenase, to form a plurality of isolated MSCs.

### Isolation

In some embodiments, MSCs are isolated from other bound cells (e.g., epithelial cells) and components of an extracellular matrix (e.g., stromal extracellular matrix) in a tissue sample by contacting the tissue sample with a protease. In some embodiments, MSCs are isolated from other bound cells and components of an extracellular matrix (e.g., stromal extracellular matrix) in the tissue sample by contacting the tissue sample with a protease that degrades and/or hydrolyzes components of the interstitial space (e.g., stroma) but not components of the basement membrane (e.g., collagens, heparan sulfate proteoglycans, laminin, and nidogen). In some embodiments, MSCs are isolated from other bound cells and components of an extracellular matrix (e.g., stromal extracellular matrix) in the tissue sample by contacting the tissue sample with dispase. Dispase cleaves fibronectin, collagen IV, and collagen I.

In some embodiments, MSCs are isolated from other bound cells (e.g., epithelial cells) and components of an extracellular matrix (e.g., stromal extracellular matrix) in a tissue sample by contacting the tissue sample with an enzyme that hydrolyzes and/or degrades interstitial (e.g., stromal) collagen but not basement membrane collagen. In some embodiments, MSCs are separated from other bound cells (e.g., epithelial cells) and components of an extracellular matrix (e.g., stromal extracellular matrix) in a tissue sample by contacting the tissue sample with a collagenase. In some embodiments, MSCs are separated from other bound cells (e.g., epithelial cells) and components of an extracellular matrix (e.g., stromal extracellular matrix) in a tissue sample by contacting the tissue sample with collagenase A, collagenase B, collagenase D, and/or a combination thereof. In some embodiments, MSCs are separated from other bound cells (e.g., epithelial cells) and components of an extracellular matrix (e.g., stromal extracellular matrix) in a tissue sample by contacting the tissue sample with collagenase A.

In some embodiments, MSCs are isolated from other bound cells (e.g., epithelial cells) and components of an extracellular matrix in the tissue sample by contacting the tissue sample with dispase and a collagenase. In some embodiments, MSCs are isolated from other bound cells (e.g., epithelial cells) and components of an extracellular matrix in the tissue sample by contacting the tissue sample with dispase and collagenase A.

### Expansion

In some embodiments, isolated MSCs are subjected to a first expansion. In some embodiments, the first expansion occurs on a coated and/or 2-dimensional substrate. In some embodiments, the substrate is coated in composition that mimics the basement membrane and/or comprises components of the basement membrane, such as such as laminin, type IV collagen and heparan sulfate proteoglycan. In some embodiments, the substrate is coated in a gelatinous protein mixture secreted by Engelbreth-Holm-Swarm (EHS) mouse sarcoma cells. In some embodiments, the substrate is coated in Matrigel. In some embodiments, the 2-dimensional substrate mimics the basement membrane and/or comprises components of the basement membrane, such as such as laminin, type IV collagen and heparan sulfate proteoglycan. In some embodiments, the 2-dimensional substrate is a gelatinous protein mixture secreted by Engelbreth-Holm-Swarm (EHS) mouse sarcoma cells. In some embodiments, the 2-dimensional substrate is Matrigel. In some embodiments, expansion on a coated and/or 2-dimensional substrate (e.g., a Matrigel coated and/or 2D substrate) results in proliferation of MSCs. In some embodiments, expansion on a coated and/or 2-dimensional substrate (e.g., a Matrigel coated and/or 2D substrate) results in proliferation of MSCs and transient loss of expression of embryonic stem cell (ESC) markers.

In some embodiments, isolated MSCs are subjected to a second expansion after the first expansion. In some embodiments, the second expansion occurs on a 3-dimensional substrate. In some embodiments, the 3-dimensional substrate mimics the basement membrane and/or comprises components of the basement membrane, such as such as laminin, type IV collagen and heparan sulfate proteoglycan. In some embodiments, the 3-dimensional substrate is a gelatinous protein mixture secreted by Engelbreth-Holm-Swarm (EHS) mouse sarcoma cells. In some embodiments, the 3-dimensional substrate is Matrigel. In some embodiments, expansion on a 3-dimensional substrate (e.g., a Matrigel 3D substrate) results in the MSCs from the first expansion regaining expression of ESC markers. In some embodiments, expansion of MSCs on a 3-dimensional substrate (e.g., a Matrigel 3D substrate) in the presence epithelial cells of results in the formation of epithelial/MSC spheres/aggregates.

### Medium

In some embodiments, isolation of the MSCs takes place in embryonic stem cell medium, supplemented hormonal epithelial medium, and/or a combination thereof. In some embodiments, isolation of the MSCs takes place in embryonic stem cell medium. In some embodiments, isolation of the MSCs takes place in human embryonic stem cell medium. In some embodiments, isolation of the MSCs takes place in human embryonic stem cell medium supplemented with bFGF and LIF.

In some embodiments, the first expansion takes place in culture comprising embryonic stem cell medium, supplemented hormonal epithelial medium, and/or a combination thereof. In some embodiments, the first expansion takes place in culture comprising embryonic stem cell medium. In some embodiments, the first expansion takes place in culture comprising human embryonic stem cell medium. In some embodiments, the first expansion takes place in culture comprising human embryonic stem cell medium supplemented with bFGF and LIF. In some embodiments, the first expansion takes place in culture further comprising an inhibitor of Rho-associated kinase (ROCK inhibitor). In some embodiments, use of DMEM medium (containing 10% FBS) for the first culture results in irreversible loss of ESC markers.

In some embodiments, the second expansion takes place in culture comprising embryonic stem cell medium, supplemented hormonal epithelial medium, and/or a combination thereof. In some embodiments, the second expansion takes place in culture comprising embryonic stem cell medium. In some embodiments, the second expansion takes place in culture comprising human embryonic stem cell medium. In some embodiments, the second expansion takes place in culture comprising human embryonic stem cell medium supplemented with bFGF and LIF. In some embodiments, the second expansion takes place in culture further comprising an inhibitor of Rho-associated kinase (ROCK inhibitor).

### Adipose Derived Stromal Cells (ASCs)

Disclosed herein, in certain embodiments, are methods of isolating and expanding adipose derived stromal cells. In some embodiments, the methods comprise (a) separating a plurality of ASCs from other bound cells and components of an extracellular matrix in a tissue sample, to form a plurality of isolated multipotent cells; (b) expanding at least one of the plurality of isolated ASCs in a first culture comprising a suitable 2-dimensional substrate without passing the Hayflick limit to form a plurality of expanding ASCs; and (c) isolating and expanding at least one expanding ASC from the plurality of expanding ASCs in a second culture comprising a suitable 3-dimensional substrate, to generate a population of expanded ASCs.

In some embodiments, the methods comprise (a) separating a plurality of ASCs from other bound cells and components of an interstitial extracellular matrix in a tissue sample, to form a plurality of isolated ASCs, wherein the plurality of ASCs are not separated from basement membrane; (b) expanding at least one of the plurality of isolated ASCs in a first culture comprising a suitable 2-dimensional substrate without passing the Hayflick limit to form a plurality of expanding ASCs; and (c) isolating and expanding at least one expanding multipotent cell from the plurality of expanding ASCs in a second culture comprising a suitable 3-dimensional substrate, to generate a population of expanded ASCs.

In some embodiments, the methods comprise (a) contacting a tissue sample with a collagenase to separate a plurality of ASCs from other bound cells and components of an extracellular matrix in a tissue sample, to form a plurality of isolated ASCs; (b) expanding at least one of the plurality of isolated ASCs in a first culture comprising a suitable 2-dimensional substrate without passing the Hayflick limit to form a plurality of expanding ASCs; and (c) isolating and expanding at least one expanding multipotent cell from the plurality of expanding ASCs in a second culture comprising a suitable 3-dimensional substrate, to generate a population of expanded ASCs.

In some embodiments, the methods comprise (a) contacting a tissue sample with dispase and a collagenase to separate a plurality of ASCs from other bound cells and components of an extracellular matrix in a tissue sample, to form a plurality of isolated ASCs; (b) expanding at least one of the plurality of isolated ASCs in a first culture comprising a suitable 2-dimensional substrate without passing the Hayflick limit to form a plurality of expanding ASCs; and (c) isolating and expanding at least one expanding multipotent cell from the plurality of expanding ASCs in a second culture comprising a suitable 3-dimensional substrate, to generate a population of expanded ASCs.

In some embodiments, the methods comprise (a) contacting a tissue sample with a collagenase to separate a plurality of ASCs from other bound cells and components of an extracellular matrix in a tissue sample, to form a plurality of isolated ASCs, wherein the collagenase degrades interstitial collagen but not basement membrane collagen; (b) expanding at least one of the plurality of isolated ASCs in a first culture comprising a suitable 2-dimensional substrate without passing the Hayflick limit to form a plurality of expanding ASCs; and (c) isolating and expanding at least one expanding multipotent cell from the plurality of expanding ASCs in a second culture comprising a suitable 3-dimensional substrate, to generate a population of expanded ASCs.

In some embodiments, the methods comprise (a) contacting a tissue sample with dispase and a collagenase to separate a plurality of ASCs from other bound cells and components of an extracellular matrix in a tissue sample, to form a plurality of isolated ASCs, wherein the dispase and collagenase degrade interstitial components of the extracellular membrane but not basement membrane components; (b) expanding at least one of the plurality of isolated ASCs in a first culture comprising a suitable 2-dimensional substrate without passing the Hayflick limit to form a plurality of expanding ASCs; and (c) isolating and expanding at least one expanding multipotent cell from the plurality of expanding ASCs in a second culture comprising a suitable 3-dimensional substrate, to generate a population of expanded ASCs.

In some embodiments, the methods comprise expanding a plurality of ASCs, comprising: expanding at least one isolated multipotent cell in a culture comprising a suitable coated and/or 2-dimensional substrate without passing the Hayflick limit, to form a plurality of expanding ASCs, wherein the 2D substrate comprises a gelatinous protein mixture secreted by Engelbreth-Holm-Swarm (EHS) mouse sarcoma cells. In some embodiments, the methods further comprise expanding at least one expanding multipotent cell in a culture comprising a suitable 3-dimensional substrate, to generate a population of expanded ASCs, wherein the 3D substrate comprises a gelatinous protein mixture secreted by Engelbreth-Holm-Swarm (EHS) mouse sarcoma cells. In some embodiments, the methods contacting a tissue sample comprising a plurality of ASCs with a collagenase, to form a plurality of isolated ASCs.

In some embodiments, the methods comprise expanding a plurality of ASCs, comprising: expanding at least one expanding multipotent cell in a culture comprising a suitable 3-dimensional substrate, to generate a population of expanded ASCs, wherein the 3D substrate comprises a gelatinous protein mixture secreted by Engelbreth-Holm-Swarm (EHS) mouse sarcoma cells. In some embodiments, the methods further comprise expanding at least one isolated multipotent cell in a culture comprising a suitable coated and/or 2-dimensional substrate without passing the Hayflick limit, to form a plurality of expanding ASCs, wherein the 2D substrate comprises a gelatinous protein mixture secreted by Engelbreth-Holm-Swarm (EHS) mouse sarcoma cells. In some embodiments, the methods further comprise contacting a tissue sample comprising a plurality of ASCs with a collagenase, to form a plurality of isolated ASCs.

### Isolation

Current methods of isolating ASCs involves the following steps: (1) digesting adipose tissue with collagenase I in DMEM/10% FBS, (2) separating the stromal vascular fraction (SVF) cells, and discarding the floating cells that contain mature adipose cells, and (3) filtering the SVF via a 250 μm mesh filter and collecting cell flow through. Problematically, collecting the cell flow through results in the loss of any cells attached to basement membrane. As discussed above, many multipotent cells (especially, MSCs such as ASCs) are attached to basement membrane. Thus, the current methods of isolating ASCs results in the loss of a significant fraction of ASCs.

Disclosed herein, in certain embodiments, are novel methods of isolating ASCs. In some embodiments, the methods of isolating ASCs comprise (1) digesting adipose tissue with collagenase, to create digested adipose tissue; (2) separating the stromal vascular fraction (SVF) cells of the digested adipose tissue from other bound cells (e.g., floating cells that contain mature adipose cells), to created isolated SVF; and (3) isolating ASCs attached to basement membrane other bound cells and components of an extracellular matrix in the isolated SVF. In some embodiments, isolation of the ASCs takes place in human embryonic stem cell medium supplemented with bFGF and LIF (MESCM).

In some embodiments, isolating ASCs attached to basement membrane comprises filtering the SVF via a 250 μm mesh filter and collecting the non-cell flow through.

In some embodiments, isolating ASCs further comprises contacting the adipose tissue with a protease. In some embodiments, isolating ASCs further comprises contacting the adipose tissue with a protease that does degrade and/or hydrolyze components of the basement membrane (e.g., collagens, heparan sulfate proteoglycans, laminin, and nidogen). In some embodiments, isolating ASCs further comprises contacting the adipose tissue with dispase.

### Expansion

In some embodiments, isolated MSCs are subjected to a first expansion. In some embodiments, the first expansion occurs on a coated and/or 2-dimensional substrate. In some embodiments, the substrate is coated in composition that mimics the basement membrane and/or comprises components of the basement membrane, such as such as laminin, type IV collagen and heparan sulfate proteoglycan. In some embodiments, the substrate is coated in a gelatinous protein mixture secreted by Engelbreth-Holm-Swarm (EHS) mouse sarcoma cells. In some embodiments, the substrate is coated in Matrigel. In some embodiments, the 2-dimensional substrate mimics the basement membrane and/or comprises components of the basement membrane, such as such as laminin, type IV collagen and heparan sulfate proteoglycan. In some embodiments, the 2-dimensional substrate is a gelatinous protein mixture secreted by Engelbreth-Holm-Swarm (EHS) mouse sarcoma cells. In some embodiments, the 2-dimensional substrate is Matrigel. In some embodiments, expansion on a coated and/or 2-dimensional substrate (e.g., a Matrigel coated and/or 2D substrate) results in proliferation of MSCs. In some embodiments, expansion on a coated and/or 2-dimensional substrate (e.g., a Matrigel coated and/or 2D substrate) results in proliferation of MSCs and transient loss of expression of embryonic stem cell (ESC) markers.

In some embodiments, isolated MSCs are subjected to a second expansion after the first expansion. In some embodiments, the second expansion occurs on a 3-dimensional substrate. In some embodiments, the 3-dimensional substrate mimics the basement membrane and/or comprises components of the basement membrane, such as such as laminin, type IV collagen and heparan sulfate proteoglycan. In some embodiments, the 3-dimensional substrate is a gelatinous protein mixture secreted by Engelbreth-Holm-Swarm (EHS) mouse sarcoma cells. In some embodiments, the 3-dimensional substrate is Matrigel. In some embodiments, expansion on a 3-dimensional substrate (e.g., a Matrigel 3D substrate) results in the MSCs from the first expansion regaining expression of ESC markers. In some embodiments, expansion of MSCs on a 3-dimensional substrate (e.g., a Matrigel 3D substrate) in the presence epithelial cells of results in the formation of epithelial/MSC spheres/aggregates.

### Medium

In some embodiments, isolation of the MSCs takes place in embryonic stem cell medium, supplemented hormonal epithelial medium, and/or a combination thereof. In some embodiments, isolation of the MSCs takes place in embryonic stem cell medium. In some embodiments, isolation of the MSCs takes place in human embryonic stem cell medium. In some embodiments, isolation of the MSCs takes place in human embryonic stem cell medium supplemented with bFGF and LIF.

In some embodiments, the first expansion takes place in culture comprising embryonic stem cell medium, supplemented hormonal epithelial medium, and/or a combination thereof. In some embodiments, the first expansion takes place in culture comprising embryonic stem cell medium. In some embodiments, the first expansion takes place in culture comprising human embryonic stem cell medium. In some embodiments, the first expansion takes place in culture comprising human embryonic stem cell medium supplemented with bFGF and LIF. In some embodiments, the first expansion takes place in culture further comprising an inhibitor of Rho-associated kinase (ROCK inhibitor). In some embodiments, use of DMEM medium (containing 10% FBS) for the first culture results in irreversible loss of ESC markers.

In some embodiments, the second expansion takes place in culture comprising embryonic stem cell medium, supplemented hormonal epithelial medium, and/or a combination thereof. In some embodiments, the second expansion takes place in culture comprising embryonic stem cell medium. In some embodiments, the second expansion takes place in culture comprising human embryonic stem cell medium. In some embodiments, the second expansion takes place in culture comprising human embryonic stem cell medium supplemented with bFGF and LIF. In some embodiments, the second expansion takes place in culture further comprising an inhibitor of Rho-associated kinase (ROCK inhibitor).

### Isolated Multipotent Cell Cultures

Also provided herein is a multipotent cell culture made by the method comprising: (a) separating a plurality of multipotent cells (e.g., MSCs; (e.g., ASCs)) from other bound cells and components of an extracellular matrix in a tissue sample, to form a plurality of isolated multipotent cells; (b) expanding at least one of the plurality of isolated multipotent cells in a first culture comprising a suitable 2-dimensional substrate without passing the Hayflick limit, to form a plurality of expanding multipotent cells; and (c) isolating and expanding at least one stem cell from the plurality of expanding multipotent cells in a second culture comprising a suitable 3-dimensional substrate, to generate a population of expanded multipotent cells (e.g., MSCs; (e.g., ASCs)).

### Isolation

In some embodiments, multipotent cells (e.g., MSCs) are isolated from other bound cells (e.g., epithelial cells) and components of an extracellular matrix (e.g., stromal extracellular matrix) in a tissue sample by contacting the tissue sample with a protease. In some embodiments, the multipotent cells (e.g., MSCs) are isolated from other bound cells and components of an extracellular matrix (e.g., stromal extracellular matrix) in the tissue sample by contacting the tissue sample with a protease that degrades and/or hydrolyzes components of the interstitial space (e.g., stroma) but not components of the basement membrane (e.g., collagens, heparan sulfate proteoglycans, laminin, and nidogen). In some embodiments, the multipotent cells (MSCs) are isolated from other bound cells and components of an extracellular matrix (e.g., stromal extracellular matrix) in the tissue sample by contacting the tissue sample with dispase. Dispase cleaves fibronectin, collagen IV, and collagen I.

In some embodiments, the multipotent cells (MSCs) are isolated from other bound cells (e.g., epithelial cells) and components of an extracellular matrix (e.g., stromal extracellular matrix) in a tissue sample by contacting the tissue sample with an enzyme that hydrolyzes and/or degrades interstitial (e.g., stromal) collagen but not basement membrane collagen. In some embodiments, the multipotent cells (e.g., MSCs) are separated from other bound cells (e.g., epithelial cells) and components of an extracellular matrix (e.g., stromal extracellular matrix) in a tissue sample by contacting the tissue sample with a collagenase. In some embodiments, the multipotent cells (e.g., MSCs) are separated from other bound cells (e.g., epithelial cells) and components of an extracellular matrix (e.g., stromal extracellular matrix) in a tissue sample by contacting the tissue sample with collagenase A, collagenase B, collagenase D, and/or a combination thereof. In some embodiments, the multipotent cells (e.g., MSCs) are separated from other bound cells (e.g., epithelial cells) and components of an extracellular matrix (e.g., stromal extracellular matrix) in a tissue sample by contacting the tissue sample with collagenase A.

In some embodiments, the multipotent cells (MSCs) are isolated from other bound cells (e.g., epithelial cells) and components of an extracellular matrix in the tissue sample by contacting the tissue sample with dispase and a collagenase. In some embodiments, the multipotent cells (MSCs) are isolated from other bound cells (e.g., epithelial cells) and components of an extracellular matrix in the tissue sample by contacting the tissue sample with dispase and collagenase A.

### Expansion

In some embodiments, isolated multipotent cells (e.g., MSCs) are subjected to a first expansion. In some embodiments, the first expansion occurs on a coated and/or 2-dimensional substrate. In some embodiments, the substrate is coated in composition that mimics the basement membrane and/or comprises components of the basement membrane, such as such as laminin, type IV collagen and heparan sulfate proteoglycan. In some embodiments, the substrate is coated in a gelatinous protein mixture secreted by Engelbreth-Holm-Swarm (EHS) mouse sarcoma cells. In some embodiments, the substrate is coated in Matrigel. In some embodiments, the 2-dimensional substrate mimics the basement membrane and/or comprises components of the basement membrane, such as such as laminin, type IV collagen and heparan sulfate proteoglycan. In some embodiments, the 2-dimensional substrate is a gelatinous protein mixture secreted by Engelbreth-Holm-Swarm (EHS) mouse sarcoma cells. In some embodiments, the 2-dimensional substrate is Matrigel. In some embodiments, expansion on a coated and/or 2-dimensional substrate (e.g., a Matrigel coated and/or 2D substrate) results in proliferation of multipotent cells (e.g., MSCs). In some embodiments, expansion on a coated and/or 2-dimensional substrate (e.g., a Matrigel coated and/or 2D substrate) results in proliferation of multipotent cells (e.g., MSCs) and transient loss of expression of embryonic stem cell (ESC) markers.

In some embodiments, isolated multipotent cells (e.g., MSCs) are subjected to a second expansion after the first expansion. In some embodiments, the second expansion occurs on a 3-dimensional substrate. In some embodiments, the 3-dimensional substrate mimics the basement membrane and/or comprises components of the basement membrane, such as such as laminin, type IV collagen and heparan sulfate proteoglycan. In some embodiments, the 3-dimensional substrate is a gelatinous protein mixture secreted by Engelbreth-Holm-Swarm (EHS) mouse sarcoma cells. In some embodiments, the 3-dimensional substrate is Matrigel. In some embodiments, expansion on a 3-dimensional substrate (e.g., a Matrigel 3D substrate) results in the cells from the first expansion regaining expression of ESC markers. In some embodiments, expansion of MSCs on a 3-dimensional substrate (e.g., a Matrigel 3D substrate) in the presence epithelial cells of results in the formation of epithelial/MSC spheres/aggregates.

### Medium

In some embodiments, isolation of the multipotent cells takes place in embryonic stem cell medium, supplemented hormonal epithelial medium, and/or a combination thereof. In some embodiments, isolation of the multipotent cells takes place in embryonic stem cell medium. In some embodiments, isolation of the multipotent cells takes place in human embryonic stem cell medium. In some embodiments, isolation of the multipotent cells takes place in human embryonic stem cell medium supplemented with bFGF and LIF.

In some embodiments, the first expansion takes place in culture comprising embryonic stem cell medium, supplemented hormonal epithelial medium, and/or a combination thereof. In some embodiments, the first expansion takes place in culture comprising embryonic stem cell medium. In some embodiments, the first expansion takes place in culture comprising human embryonic stem cell medium. In some embodiments, the first expansion takes place in culture comprising human embryonic stem cell medium supplemented with bFGF and LIF. In some embodiments, the first expansion takes place in culture further comprising an inhibitor of Rho-associated kinase (ROCK inhibitor). In some embodiments, use of DMEM medium (containing 10% FBS) for the first culture results in irreversible loss of ESC markers.

In some embodiments, the second expansion takes place in culture comprising embryonic stem cell medium, supplemented hormonal epithelial medium, and/or a combination thereof. In some embodiments, the second expansion takes place in culture comprising embryonic stem cell medium. In some embodiments, the second expansion takes place in culture comprising human embryonic stem cell medium. In some embodiments, the second expansion takes place in culture comprising human embryonic stem cell medium supplemented with bFGF and LIF. In some embodiments, the second expansion takes place in culture further comprising an inhibitor of Rho-associated kinase (ROCK inhibitor).

### Uses of Isolated Multipotent Cell Populations

For any or all of the following uses, the multipotent cells are administered by any suitable means. For example, they are administered by infusion (e.g., into an organ or bone marrow) or they are administered by a wound covering or bandage.

In some embodiments, the expanded multipotent cells obtained by any of the methods described herein are used for transplantation into an individual in need thereof. In some embodiments, the cells are isolated from one individual and transplanted into another individual. Such transplantation may be used to regenerate a damaged tissue.

In some embodiments, the expanded multipotent cells disclosed herein are transplanted into the bone marrow of an individual whose bone marrow does not produce an adequate supply of stem cells. In some embodiments, the expanded multipotent cells disclosed herein are transplanted into an individual whose bone marrow does not produce an adequate supply of white blood cells. In some embodiments, the expanded multipotent cells disclosed herein are transplanted into an individual whose bone marrow does not produce an adequate supply of red blood cells. In some embodiments, the expanded multipotent cells disclosed herein are transplanted into an individual whose bone marrow does not produce an adequate supply of platelets. In some embodiments, the expanded multipotent cells disclosed herein are transplanted into an individual that suffers from anemia. In some embodiments, the expanded multipotent cells disclosed herein are transplanted into the bone marrow of an individual following chemotherapy and/or radiation therapy.

In some embodiments, the expanded multipotent cells disclosed herein are transplanted into an individual suffering from neurological damage. In some embodiments, the expanded multipotent cells disclosed herein are transplanted into an individual to regenerate neurons.

In some embodiments, the expanded multipotent cells disclosed herein are transplanted into an individual suffering from a neurodegenerative disease. In some embodiments, the expanded multipotent cells disclosed herein are transplanted into an individual to treat Parkinson's disease. In some embodiments, the expanded multipotent cells disclosed herein are transplanted into an individual to treat Alzheimer's disease.

In some embodiments, the expanded multipotent cells disclosed herein are transplanted into an individual to treat a stroke.

In some embodiments, the expanded multipotent cells disclosed herein are transplanted into an individual to treat traumatic brain injury.

In some embodiments, the expanded multipotent cells disclosed herein are transplanted into the spinal cord of an individual suffering from a spinal cord injury. In some embodiments, the expanded multipotent cells disclosed herein are transplanted into the spinal cord of an individual to treat paralysis (e.g., due to a spinal cord injury).

In some embodiments, the expanded multipotent cells disclosed herein are transplanted into an individual to treat amyotrophic lateral sclerosis (ALS).

In some embodiments, the expanded multipotent cells disclosed herein are transplanted into an individual to treat heart damage. In some embodiments, the expanded multipotent cells disclosed herein are transplanted into an individual to treat/regenerate damaged heart muscle. In some embodiments, the expanded multipotent cells disclosed herein are transplanted into an individual to treat/regenerate damaged blood vessels (i.e., to promote angiogenesis).

In some embodiments, the expanded multipotent cells disclosed herein are transplanted into an individual to treat baldness.

In some embodiments, the expanded multipotent cells disclosed herein are transplanted into an individual to regenerate missing teeth.

In some embodiments, the expanded multipotent cells disclosed herein are transplanted into an individual to treat deafness. In some embodiments, the expanded multipotent cells disclosed herein are transplanted into an individual to regenerate hair cells of the auditory system.

In some embodiments, the expanded multipotent cells disclosed herein are transplanted into an individual to treat blindness.

In some embodiments, the expanded multipotent cells disclosed herein are transplanted into an individual to treat a skin wound. In some embodiments, the expanded multipotent cells disclosed herein are transplanted into an individual to treat a chronic skin wound. In some embodiments, the expanded multipotent cells disclosed herein are administered to the individual via a wound covering or bandage.

In some embodiments, the expanded multipotent cells disclosed herein are used as niche cells to support the growth of epithelial progenitor cells. In some embodiments, the expanded multipotent cells disclosed herein are used as niche cells in vivo to support the growth of epithelial progenitor cells, for example to treat a disease, disorder and/or condition characterized by epithelial progenitor cell failure. In some embodiments, the expanded multipotent cells disclosed herein are used as niche cells to support the growth of epithelial progenitor cells in vitro (i.e., in cell culture). In some embodiments, the expanded multipotent cells disclosed herein are used as niche cells to support the growth of epithelial progenitor cells into tissue grafts.

In some embodiments, the expanded multipotent cells disclosed herein are used to treat an autoimmune disease. In some embodiments, the expanded multipotent cells disclosed herein are administered to an individual with an autoimmune disease. In some embodiments, the autoimmune disease is selected from diabetes mellitus, psoriasis, Crohn's disease, or any combination thereof.

In some embodiments, the expanded multipotent cells disclosed herein are used to treat or prevent transplant rejection, for example they are administered to an individual receiving a bone marrow transplant, a kidney transplant, a liver transplant, a lung transplant. In some embodiments, the expanded multipotent cells disclosed herein are administered to the individual with psoriasis via a wound covering or bandage. In some embodiments, the expanded multipotent cells disclosed herein are used to treat or prevent Graft-versus-Host disease.

In some embodiments, the expanded multipotent cells disclosed herein are transplanted into an individual to treat idiopathic pulmonary fibrosis.

In some embodiments, the expanded multipotent cells disclosed herein are transplanted into an individual to treat a cancer.

In some embodiments, the expanded multipotent cells disclosed herein are transplanted into an individual to treat aplastic anemia.

In some embodiments, the expanded multipotent cells disclosed herein are transplanted into an individual to reconstitute the immune system of an HIV positive individual.

In some embodiments, the expanded multipotent cells disclosed herein are transplanted into an individual to treat Alzheimer's Disease.

In some embodiments, the expanded multipotent cells disclosed herein are transplanted into an individual to treat liver cirrhosis.

In some embodiments, the expanded multipotent cells disclosed herein are transplanted into an individual to treat multiple sclerosis.

In some embodiments, the expanded multipotent cells disclosed herein are transplanted into an individual to treat an inflammatory disorder.

In some embodiments, the expanded multipotent cells disclosed herein are transplanted into an individual to generate or regenerate epithelial tissue. In some embodiments, the expanded multipotent cells disclosed herein are transplanted into an individual to generate or regenerate skin, bone, teeth or hair.

### EXAMPLES

### Example 1

### Collagenase Alone can, but Dispose Alone Cannot, Isolate Limbal Stromal Stem Cells

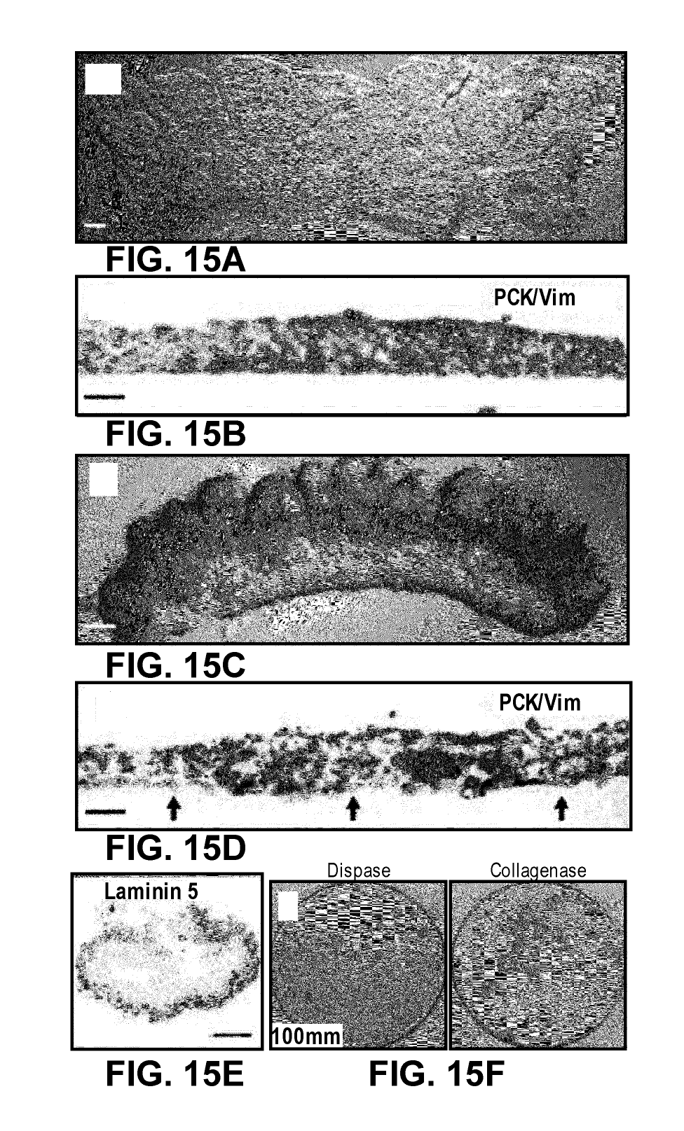

As shown in Tables 1-3, both dispase and trypsin/EDTA have been used to isolate limbal epithelial SCs. FIG. 15 shows that dispase alone does not isolate the mesenchymal cells (Vim+ but PCK−). The present inventors disclose a novel isolation to isolate clusters consisting of not only the entire limbal epithelial progenitor cells but also their closely associated mesenchymal cells by the use of enzymatic digestion of collagenase. This is because dispase degrades the basement membrane collagens while collagenase degrades interstitial collagens but preserves the basement membrane matrix. Thus, further enrichment to isolate these mesenchymal cells can be achieved by removing limbal epithelial cells by dispase followed by collagenase, a method called D/C method.

Specifically, human limbal tissue is cut into 12 one-clock-hour segments by incisions made at 1 mm within and beyond the anatomic limbus. For digestion with collagenase alone, the segment is digested in 1 mg/ml collagenase A at 37 C, 18 h. For D/C method, the segment is digested in 10 mg/ml of dispase 4 C for 16 h first before being put in 1 mg/ml collagenase A at 37 C, 18 h. After digestion with the D/C method, there are clusters of cells floating in the medium, called D/C cells, while the residual stromal cells (called RSC cells) appear as single adherent cells on plastic.

### Example 2

### Isolation and Expansion of Characterization of the Phenotype Characterized of hAMSC with or without 2D MATRIGEL and hAMEC from AM and UC, Respectively, after Expanded in Three Different Medium with or without 2-D MATRIGEL® and with or without a ROCK Inhibitor

### Experimental Design

One (1) cm pieces of human amniotic membrane (HAM) and umbilical cord (HUC) are cryopreserved for cross sectioned. Precut 4×4 cm2 HAM are subjected to 0.25% trypsin/EDTA (T/E) at 37° C. for 5 min and then digested with 210 mg/ml of dispase 30-60 mins at 37° C. on a shaker, and the remaining stromal tissue is subjected to 2 mg/ml collagenase with HAase (200 ug/ml) in digestion medium at 37° C. for 216 h. For hUCEC, arteries and veins are removed by forceps then 5 cm2 of UC are subjected to 2 mg/ml of dispase at 40-60 mins at 37° C. followed by 2 mg/ml collagenase with HAase (200 ug/ml) in for 2-3 16 h at 37° C. Retrieved epithelial sheet are subjected TrypLE for 10 mins. Alternatively, AM tissues are digested with 2 mg/ml collagenase with HAase (200 ug/ml) for 16 h at 37° C. Retrieved epithelia sheets are transferred and subjected to 10 mg/ml of dispase 20 mg/ml at 37° C. for 20 minutes, Retrieved epithelial sheet from both isolation methods Dispase/Coll are subjected to TrypLE for 105 mins. The retrieved hAMSC are collected to compare mRNA level for expression of angiogenic markers.

Double stainings of PCK and Vim confirm <1% of PCK were present in both C/D and D/C methods. C/D yields a higher percentage of angiogenic progenitors than D/C. Immunstaining showed C/D derived cells positive expressions of angiogenic markers including (FLK-1, PDGFRβ, NG2, α-SMA, vWF, CD31). The expression of FLK-1 but not other markers reported isolated from fresh hAMSC. C/D derived cells confirmed low CD34 positive cells were detected. C/D derived cells showed strong S100A4, a marker of myofibroblasts but no expression of SMMHC, a marker of smooth muscle cells. mRNA confirmed the expressions of ES (Oct4, Nanog, Sox2), angiogenic (FLK1, PDGFR-β, NG2, α-SMA, CD146, CD31) were significantly higher in C/D than D/C method.

Cells are cultured in DMEM/10% FBS, SHEM or modified ESCM on plastic with or without 2-D MATRIGEL® at density of 1.27×1045/cm2 for hAMEC (see, Chen, 2007) and 15×1054 cm2 for hAMSC (see, Hua-Tao, p217) cells in a 24-well plate in triplicate or in a 6-well plate for protein and RNA (estimated to be around 30 to 40% confluence). One would understand that the culture conditions (e.g., seeding density and well size) are chosen so that enough lysate is collected for later uses at multiple time points.

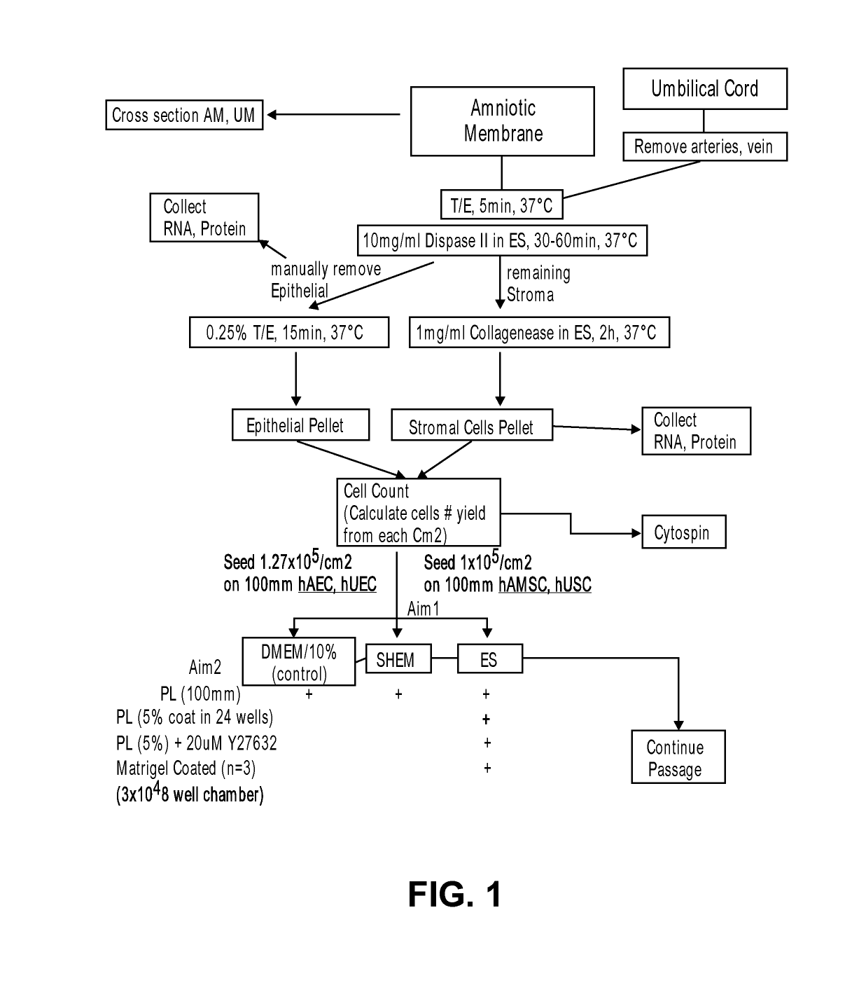

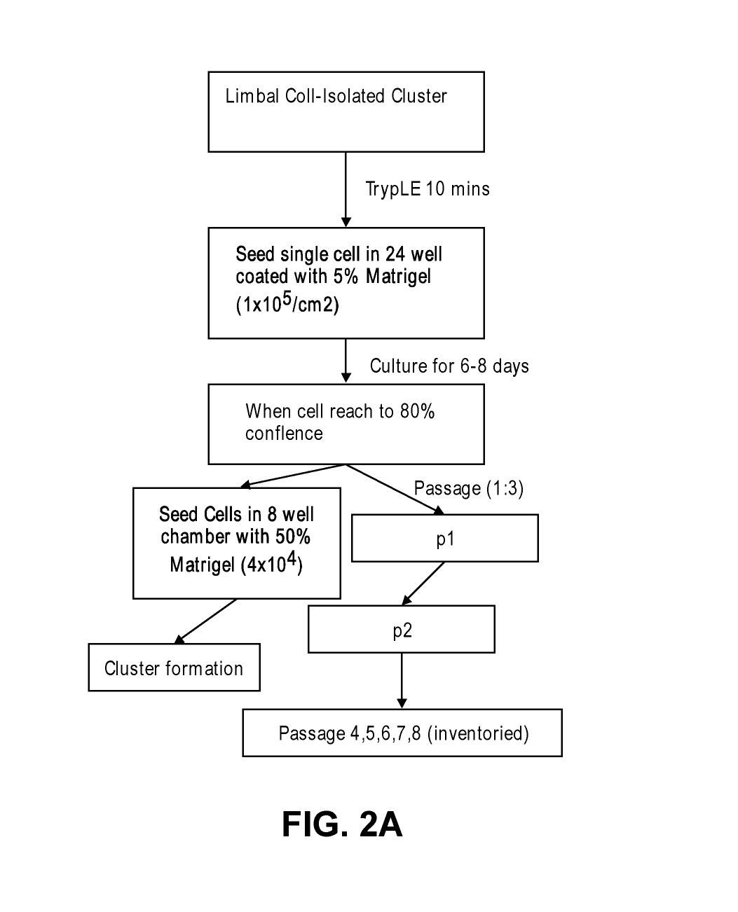

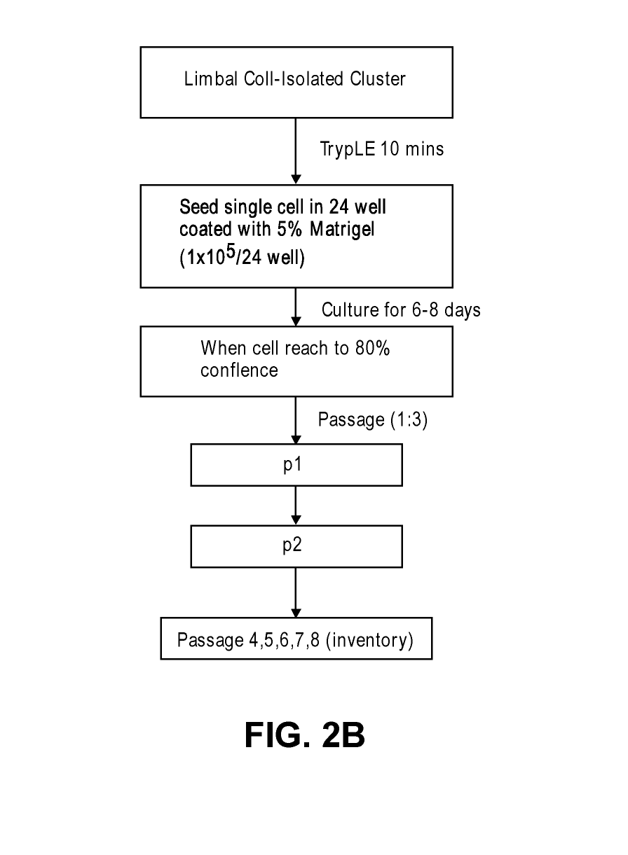

The culture in 2-D MATRIGEL® with MESCM is also be added with or without ROCK inhibitor (20 μM). For each passage, cells are seeded at the density 3×10⁴/8 well chamber on 3-D MATRIGEL® (1:1) in MESCM consists of DMEM/F12 (1:1) 10% Knockout serum, 2-mercaptoethanol bFGF (4 ng/ml), LIF (10 ng/ml) and ITS. Cell count and % yield from each isolation are performed for determination of the cell doubling time. Cell lysate of hAMSC and hAMEC are collected direct from enzymatic digestion or from different culture medium to measure the protein and RNA levels, and stored for future uses. At each passage, cell lysates are collected for proteins and mRNA for expression of ESC markers, Vim (EMT), miRNAs. FIG. 1 illustrates an exemplary method as described herein which may be used to isolate stem cells. Tables 6-9 illustrate exemplary templates for cultures of hAMEC, hAMSC, hUCEC, and hUCSC, respectively.

TABLE 6
In vitro Culture of hAMEC

| | | 1 | 2 | 3 | 4 | 5 | 6 | 7 |
|---|---|---|---|---|---|---|---|---|
| Medium | DMEM/PL | + | | | | | | |
| | SHEM/PL | | + | | | | | |
| | ESCM/PL | | | + | | | | |
| | ESCM + Y27632/PL | | | | + | | | |
| Substrate | ESCM | | | | | + | | |
| (5% MG) | ESCM + Y27632/PL | | | | | | + | |
| | ESCM + 50% MG | | | | | | | + |

TABLE 7
In vitro Culture of hAMSC

| | | 1 | 2 | 3 | 4 | 5 | 6 | 7 |
|---|---|---|---|---|---|---|---|---|
| Medium | DMEM/PL | + | | | | | | |
| | SHEM/PL | | + | | | | | |
| | ESCMPL | | | + | | | | |
| | ESCM + Y27632/PL | | | | + | | | |
| Substrate | ESCM | | | | | + | | |
| (5% MG) | ES + Y27632/PL | | | | | | + | |
| | ES + 50% MG | | | | | | | + |

TABLE 8
In vitro Culture of hUCEC

| | | 1 | 2 | 3 | 4 | 5 | 6 | 7 |
|---|---|---|---|---|---|---|---|---|
| Medium | DMEM/PL | + | | | | | | |
| | SHEM/PL | | + | | | | | |
| | ESCM/PL | | | + | | | | |
| | ESCM + Y27632/PL | | | | + | | | |
| Substrate | ESCM | | | | | + | | |
| (5% MG) | ESCM + Y27632/PL | | | | | | + | |
| | ES + 50% MG | | | | | | | + |

TABLE 9
In vitro Culture of hUCSC

| | | 1 | 2 | 3 | 4 | 5 | 6 | 7 |
|---|---|---|---|---|---|---|---|---|
| Medium | DMEM/PL | + | | | | | | |
| | SHEM/PL | | + | | | | | |
| | ESCM/PL | | | + | | | | |
| | ESCM + Y27632/PL | | | | + | | | |
| Substrate | ESCM | | | | | + | | |
| (5% MG) | ESCM + Y27632/PL | | | | | | + | |
| | ESCM + 50% MG | | | | | | | + |

### Example 3

### Methods of Isolating and Expanding Human Limbal Stroma Niche Cells

The protocol was as follows:
1) Coat 12-Well-Plate with 5% BD Matrigel.
2) The limbal rim is cut into 12 equal segments.
3) The segments are subjected to collaganase A (1 mg/ml) in ESM medium at 37 C for 16 h.
4) Get the entire limbal epithelial SCs together with their native niche cells (NCs) by pipette.
5) Harvest the total mRNA of one cluster (D0).
6) Singlize the collagenase isolated clusters by 5 ml triple at 37° C. for 10 minutes.
7) Cells are collected by centrifuge and resuspended in expansion medium.
8) Cells are seeded in the coated 24-Well-Plate at the density of 1×10⁵/cm²
9) When reaching 80% confluent, the cells are passaged at the proportion of 1:3.
10) At P3, the isolated niche cells will be seeded onto the thick gel of MATRIGEL® at a density of 4*10⁴/well in the 8-well-chamber slide.
11) Each time before passage, the total mRNA of P0, P1, P2, P3, P4, P4 gel will be harvested.
12) Run qPCR to test the mRNA level of ESC markers and epithelial cell markers.
13) Immunostaining of ESC markers.

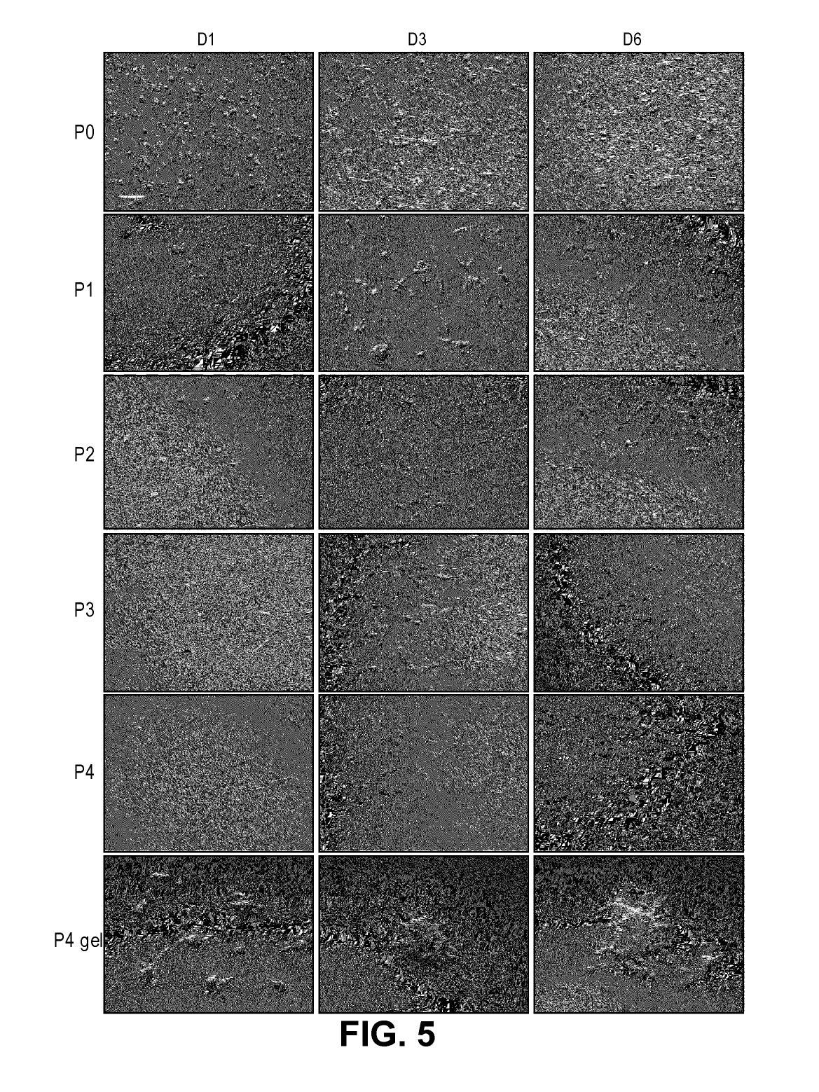

Each limbal segment yielded a limbal cluster after collagenase A digestion in the serum free expansion medium. After treated by T/E the cells were seeded on 5% MATRIGEL® (2-D) coated dish at 1×10⁵/cm. The cells are passaged at 80% confluent at 1:3. At P4, some of the cells were re-seeded back to 50% thick (3-D) MATRIGEL® (2 mm thickness). FIG. 5 illustrates niche cell isolation and purification on days D1, D3 and D6. As early as Day 1, spindle cells emerged among small round “epithelial” cells. From Passage 2 onward, almost all cells were uniformly spindle shaped. When seeded onto a thick Matrigel, Passage 4 cells turned from a spindle shape to a dendritic shape at D1 and formed aggregates at D3. Cells in the aggregate were quiescent and non-proliferating.

qPCR

The Total RNAs of each passage were collected using conventional techniques for quantitative measurement of Nanog, Sox-2, Oct-4, CD34, Rex1, and p63 using quantitative PCR (qPCR). Kits for qPCR are commercially available from, for example, Qiagen.

Immunostaining

Cytospin preparation of P4 cells were used for immunofluorescence staining using specific antibodies against Sox2, CD34 and Nanog. Immunostaining is conducted using conventional staining techniques.

Results and Conclusions

The present inventors identified that native stromal niche cells can be purified and expanded on the 2-D MATRIGEL®-coated plates (data not shown) and aggregates can be obtained when re-seeded on thick 3-D MATRIGEL®. Compared to D0 cluster immediately isolated by collagenase, qPCR revealed significantly lower expression of Nanog, Oct-4, Sox-2, and CD34 (P<0.01, n=3) at P0, P1, P2 and P3 and a gradual decline of p63 expression at P1 and disappearance by P2. It was determined that epithelial sphere growth depends on expression of ESC by niche cells.

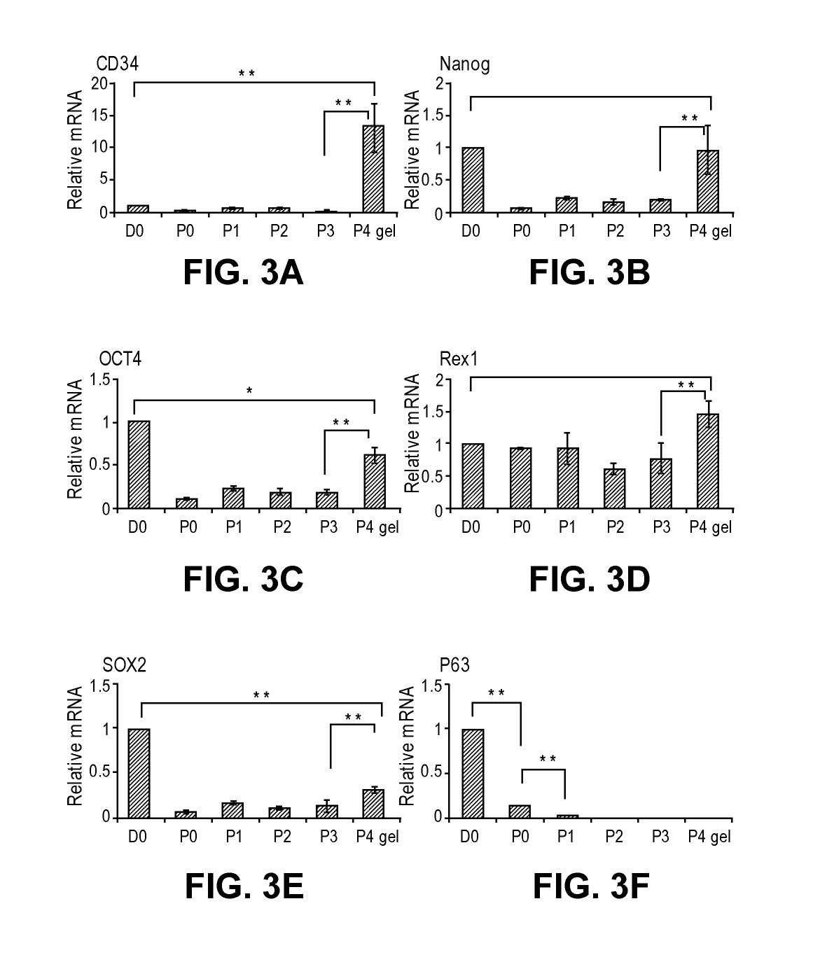

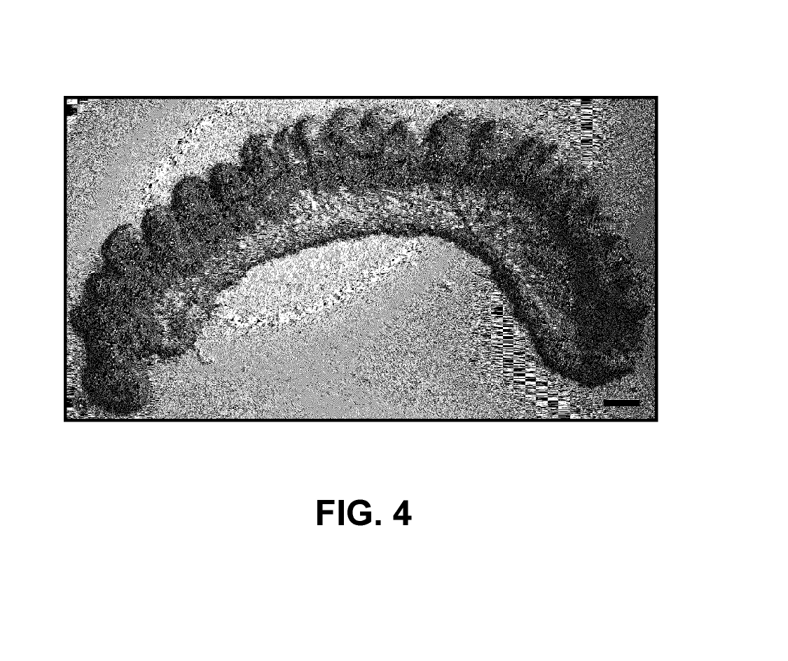

The expanded cells have the plasticity to reverse to an undifferentiated status when re-seeded on a 3-D MATRIGEL®. Niche cells expanded at the expense of losing ESC markers, when epithelial sphere growth diminished, and regained ESC Markers, when re-seeded onto thick 3-D MATRIGEL® after expansion (see, FIGS. 3A-F). When reseeded onto 3D MATRIGEL®, the mRNA expression of Nanog, Oct-4, Sox-2, CD34, and Rex1 in the aggregate significantly increased (P<0.01, n=3, compared to P3 cells, and P>0.05, compared to P0). Compared to that of the D0 cluster immediately isolated by collagenase, real-time qPCR revealed significantly lower expression of Nanog, Oct-4, Sox-2, and CD34 (P<0.01, n=3) at P0, P1, P2 and P3 and a gradual decline of expression of the epithelial cell marker p63 to undetectable by P2. Upon being seeded to a thick Matrigel, the mRNA expression of Nanog, Oct-4, Sox-2, CD34, and Rex1 in the aggregate significantly increased (P<0.01, n=3, compared to P3 cells). The level of CD34 and Rex1 expression was even higher than that in D0 clusters (P<0.01, n=3) while that of Nanog, Rex-1 had no statistical difference compared to D0 clusters (P>0.05). Immunostaining of P4 cells showed that CD34 and Sox2 and Nanog are expressed. Thus, niche cell expansion in 2D MATRIGEL® can still be reverted to express ESC markers upon reseeding in 3D MATRIGEL®.

The present inventors have identified that limbal stromal niche cells can be isolated and expanded while maintaining their phenotype. The expanded niche cells can be utilized to study limbal epithelial SC quiescence, self-renewal, and fate decision.

### Example 4

### Scale Up Expansion of hAMSC and hAMEC from AM and UC in 2-D MATRIGEL® in ESCM and Determine their Phenotype

From Example 1, the inventors learned that the in vivo phenotype of both hAMSC and hAMEC is lost when cultured in the 3 different types of medium with or without 2-D MATRIGEL®. The extent of phenotypic loss is less for cells cultured in ESCM with 2-D MATRIGEL®. The inventors expected that the phenotype of the latter is reversed to, or close to, the in vivo one when reseeded in 3-D MATRIGEL®, while the remainder will not. If the phenotypic reversal is incomplete even for the latter one, it is anticipated that addition of a ROCK inhibitor will notably improve such expression. This baseline data allows for identification of the best culturing condition (i.e., maintaining the expression of in vivo phenotype) to scale up the expansion.

Table 10 shows all the MSC phenotypic studies are detected directed from in vitro from passage 0-5 in serum containing medium.

TABLE 10

| | | | hAEC | hAMSCs |
|---|---|---|---|---|
| Measure at Passage | | BMC | In vivo | p, p4, p5 |
| ES markers | OCT4 | | + | + |
| | SSEA-3 | | | |
| | SSEA-4 | | | |
| | Tra1-60 | | | |
| | Tra1-81 | | | |
| MSC CD markers | CD13 | + | | + |
| | CD29 | + | + | + |
| | CD44 | + | | + |
| | CD49e | + | | + |
| | CD54 | + | | + |
| | CD73 | + | + | + |
| | CD90 | + | + | + |
| | CD105 | + | | + |
| | CD166 | + | + | + |
| | CD271 | + | + | + |
| | CD14 | | − | − |
| | CD31 | | − | − |
| | CD34 | | − | − |
| | CD45 | | − | − |
| MHC | MHCI | + | + | + |
| | MHCII | + | − | − |

Experimental Design

For 3-D-50% MATRIGEL® preparation, cells are seeded at 4×10⁴ per 8-well chamber slides. Cell lysate is collected from each passage as shown in Table 11.

TABLE 11

| Cell lysate | 1 | 2 | 3 | 4 | 5 | 6 | 7 | 8 |
|---|---|---|---|---|---|---|---|---|
| p0 (3D) | + | | | | | | | |
| P1 (3D) | | + | | | | | | |
| P2 (3D) | | | + | | | | | |
| P4 (3D) | | | | + | | | | |
| P6 (3D) | | | | | + | | | |
| P8 (3D) | | | | | | + | | |
| P10 (3D) | | | | | | | + | |

### Example 5

### Isolation of Entire Limbal Epithelial SCs Together with their Native Niche Cells (NCs) by Collagenase Alone

The present inventors sought to determine whether stromal niche cells be isolated by manipulating the thickness of substrate and if the phenotype of niche cells be maintained in the expansion medium constituting of DMEM/F-12 (1:1) supplemented with 10% knockout serum (Invitrogen, USA), basic-FGF 4 ng/ml insulin 5 μg/ml, transferring 5 μg/ml, sodium selenite 5 ng/ml (Sigma, USA) and human LIF 10 ng/ml (Chemicon, USA). The inventors also sought to determine whether the expanded niche cells are better than 3T3 feeder layer in supporting the limbal epithelial stem cells when co-culturing with the limbal stem cells.

The present inventors have identified a new, improved method of isolating the entire limbal epithelial SCs together with their native niche cells (NCs) by collagenase alone. The native niche cells are characterized as a phenotype with a small round shape and expression of “Embryonic Stem Cell (ESC) markers”.

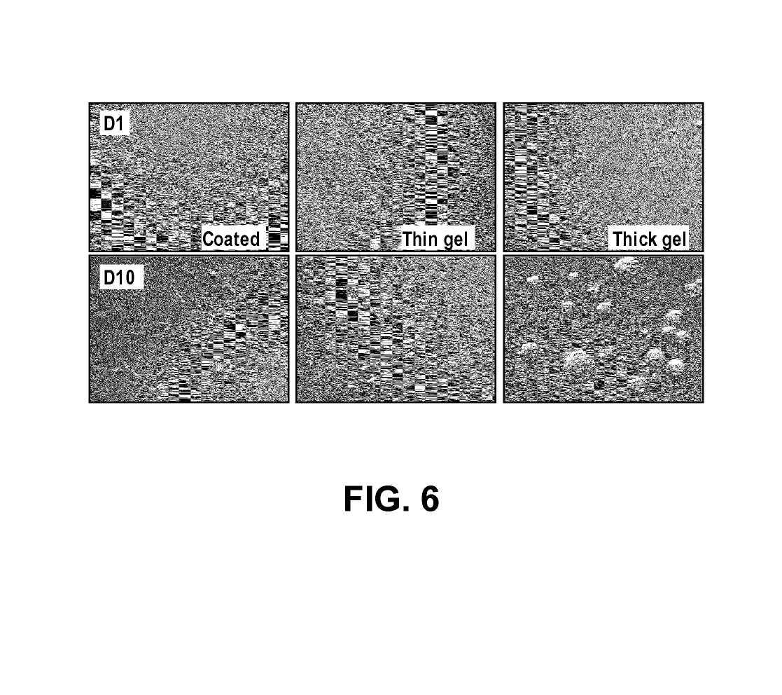

Data show that, in a new sphere culture system, vivid sphere growth occurred in the condition added with 100 μl of 50% MATRIGEL® (to yield a thick gel). However, if 35 μl of 5% MATRIGEL® was coated or 50% MATRIGEL® was applied to yield a thin gel on each 8-chamber slide, the epithelial cells cannot survive on Day 10, but instead “fibroblast-like” cells emerged (see, FIG. 6). The expansion medium can maintain the undifferentiated status of the mesenchymal cells.

### Example 6

Corneoscleral rims from 18 to 60 years old donors were obtained from the Florida Lions Eye Bank (Miami, Fla.) and managed in accordance with the declaration of Helsinki. The limbal explants were digested with Dispase II at 4° C. for 16 h to generate intact epithelial sheets or with collagenase A (Coll) at 37° C. for 18 h to generate clusters containing the entire limbal epithelial sheet with subjacent stromal cells. To enrich isolation stromal cells subjacent to limbal basal epithelial cells, we first removed the limbal epithelial sheet using Dispase and then digested the remaining stroma with collagenase, in a manner termed D/C, resulting in floating cell clusters and single cells adherent on plastic. The former, termed as D/C cells and the latter were termed as residual stromal cells (RSC). The D/C clusters were further digested with 0.25% trypsin and 1 mM EDTA (T/E) at 37° C. for 15 min to yield single cells before being seeded at the density of 1×10⁴ per cm² in 6-well plates either on coated Matrigel in ESCM containing 10 ng/ml LIF and 4 ng/ml bFGF (MESCM) or on plastic in DMEM with 10% FBS (DF). Upon 80-90% confluence, they were serially passaged at the density of 5×10³ per cm². Bone marrow-derived MSC and human corneal fibroblasts (HCF) were cultured on plastic in DF as the controls.

### Culturing in 3D Matrigel

Three dimensional (3D) Matrigel was prepared by adding 150 μl of 50% Matrigel (diluted in MESCM) per chamber of a 8-well chamber slide following incubation at 37° C. for 30 min. Single collagenase (Coll)-isolated cells, D/C cells, and RSC were seeded in 3D Matrigel and cultured for 10 days in MESCM. Single cells from resultant spheres were released by digestion with 10 mg/ml dispase II at 37° C. for 2 h followed by T/E, and mixed with red fluorescent nanocrystals pre-labeled HUVEC at a ratio of 1:1 and seeded at the density of 10⁵ cells per cm² on the surface of 3D Matrigel prepared by adding 50 μl of 100% Matrigel into 24 well plates for 30 min before use, and cultured in EGM2 to elicit vascular tube-like network as reported. HUVEC alone were seeded at the same density as the control. Single LEPC obtained by dispase-isolated limbal epithelial sheets were mixed at a ratio of 4:1 with the cells serially passaged on plastic or coated Matrigel and seeded at the total density of 5×10⁴ per cm² in 3D Matrigel. After 10 days of culture in MESCM, the resultant sphere growth was collected by digestion off Matrigel with 10 mg/ml dispase II at 37° C. for 2 h.

### CFU-F Assay

To determine the CFU-F, each group of cells was seeded at the density of 50 cells per cm² in 75 cm² plastic dishes in DF. After 12 days of culturing, cells were fixed with methanol (5 min, RT) and stained with 0.5% crystal violet in glacial acetic acid for 15 min. Resultant fibroblast-like clones were subdivided into three types according to the reported grading system, i.e., micro (5-24 cells), small (>25 cells, <2 mm), or large (>2 mm) clones. The total numbers of clones were counted and expressed as the percentage of seeded cells (%) in triplicate.

### Assays for Adipogenesis, Osteogenesis and Chondrogenesis

For assays of adipogenesis or osteogenesis, single cells were seeded at the density of 1×10⁴ cells per cm² in 24-well plastic plates in DF. After cells reached 90% confluence, the medium was switched to the Adipogenesis Differentiation Medium or the Osteogenesis Differentiation Medium (and changed every 3 days. After 21 days of culturing, cells were fixed with 4% formaldehyde and stained with Oil Red O for adipocytes or with Alizarin Red for osteocytes following the manufacturer's protocol. Cells with positive Oil Red O were counted in a total of 2,000 cells in triplicate cultures. Mineralized cells with positive Alizarin Red staining were quantified by the procedure following the manufacturer's protocol by measuring OD at 450 nm in triplicate cultures. For the chondrogenesis assay, pellets were prepared by spinning down 3×10⁵ cells and incubated in the Chondrogenesis Differentiation Medium with the medium changed every 3 days. After 28 days of culturing, pellets were fixed with 4% formaldehyde, embedded in the Optimal Cutting Temperature Compound, prepared for 6 μm frozen cross-sections, and stained with Alcian Blue.

### Immunofluorescence Staining

Single cells were prepared for cytospin using Cytofuge® at 1,000 rpm for 8 min, fixed with 4% formaldehyde for 15 min, permeabilized with 0.2% Triton X-100 in PBS for 15 min, and blocked with 2% BSA in PBS for 1 h before being incubated with primary antibodies overnight at 4° C. After washing with PBS, cytospin preparations were incubated with corresponding secondary antibodies for 1 h using appropriate isotype-matched non-specific IgG antibodies as controls. The nucleus was counterstained with Hoechst 33342 before being analyzed with a Zeiss LSM 700 confocal microscope.

### Reverse Transcription and Quantitative Real-Time Polymerase Chain Reaction (RT-qPCR)

Total RNAs were extracted by RNeasy Mini RNA Isolation Kit. A total of 1-2 μg of total RNAs was reverse-transcribed to cDNA by High Capacity cDNA Transcription Kit. RT-qPCR was carried out in a 20 μl solution containing cDNA, TaqMan Gene Expression AssayMix, and universal PCR Master Mix. The results were normalized by an internal control, i.e., glyceraldehyde-3-phosphate dehydrogenase (GAPDH). All assays were performed in triplicate for each primer set. The relative gene expression was analyzed by the comparative CT method (ΔΔC_{T}).

### Western Blot

Proteins were extracted from day 10 spheres generated by LEPC alone or mixed with other cells in RIPA buffer supplemented with proteinase inhibitors. Equal amounts of proteins determined by the BCA assay (Pierce, Rockford, Ill.) in total cell extracts were separated by 10% SDS-PAGE and transferred to nitrocellulose membranes. Membranes were then blocked with 5% (w/v) fat-free milk in TBST (50 mMTris-HCl, pH 7.5, 150 mM NaCl, 0.05% (v/v) Tween-20), followed by sequential incubation with specific primary antibodies and their respective secondary antibodies using β-actin as the loading control. The immunoreactive bands were visualized by a chemiluminescence reagent.

### Results

### Distribution of Cells Expressing Angiogenesis Markers in Limbus Stroma

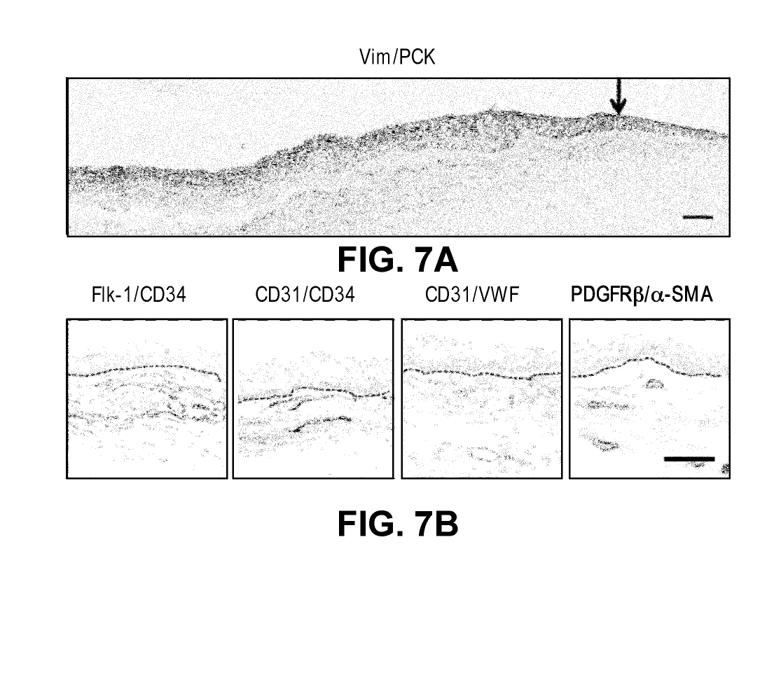

As a first step of localizing the origin of cells that carried such an angiogenesis potential, we performed double immunostaining of corneo-limbo-conjunctival sections between PCK and Vim to delineate limbal epithelial cells and underlying stromal cells, respectively (FIG. 7A). Subsequent double immunostaining between several pairs of angiogenesis markers such as Flk-1/CD34, CD31/VWF, and α-SMA/PDGFRβ also showed that some of Vim+ stromal cells expressed these markers (FIG. 7B). A closer look disclosed that cells expressing these angiogenesis markers lied not only in the perivascular location but also immediately subjacent to limbal basal epithelial cells.

### Preferential Isolation of Stromal Cells Subjacent to Limbal Basal Epithelial Cells

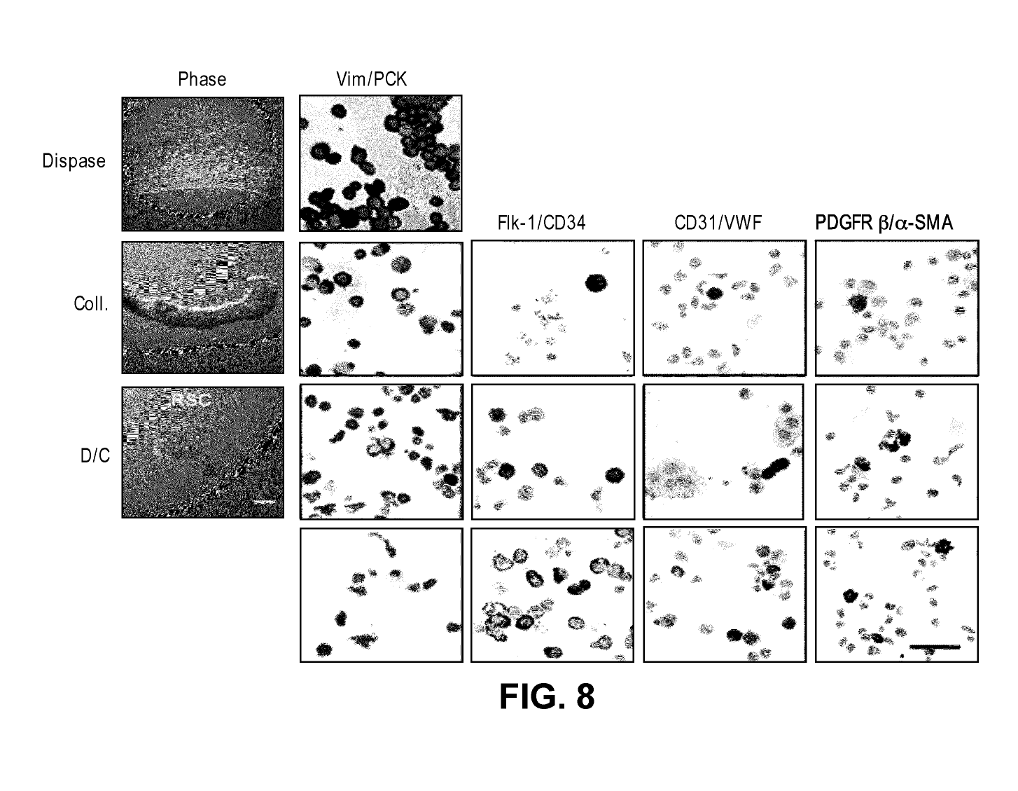

Digestion with dispase alone removed the entire limbal epithelial sheet that consisted of PCK+ epithelial cells, of which some also co-expressed Vim (FIG. 8, Dispase). In contrast, digestion with collagenase alone successfully removed PCK+ epithelial cells together with subjacent Vim+ mesenchymal cells (FIG. 8, Coll). To enrich the isolation of stromal cells subjacent to limbal basal epithelial cells, we first removed the limbal epithelial sheet by dispase digestion and then subjected the remaining stroma to collagenase digestion. This method, termed D/C digestion, yielded clusters of cells floating in the medium and residual stromal cells (RSC) adherent on the plastic dish (FIG. 8). Double immunostaining between PCK and Vim showed that approximate 80% PCK+ epithelial cells and 20% Vim+ stromal cells were present in collagenase-isolated clusters. In contrast, approximate 5% PCK+ epithelial cells and 95% Vim+ stromal cells were in D/C clusters, while all RSC cells were Vim+(FIG. 8). Double immunostaining of several angiogenesis markers and counting a total of 2,000 cells in each condition revealed that less than 1% of collagenase- or D/C-isolated Vim+ cells expressed Flk-1, CD34, CD31, or α-SMA. In RSC cells, however, more than 10% did so. Furthermore, VWF+ cells and PDGFRβ+ cells were only detected in RSC cells (FIG. 8). These results suggested that cells expressing potential angiogenesis markers were found in D/C-isolated Vim+ cells subjacent to limbal basal epithelial cells as well as in Vim+ cells in the remaining limbal stroma.

### D/C but not RSC Cells Form Spheres Containing Angiogenesis Progenitors in 3D Matrigel

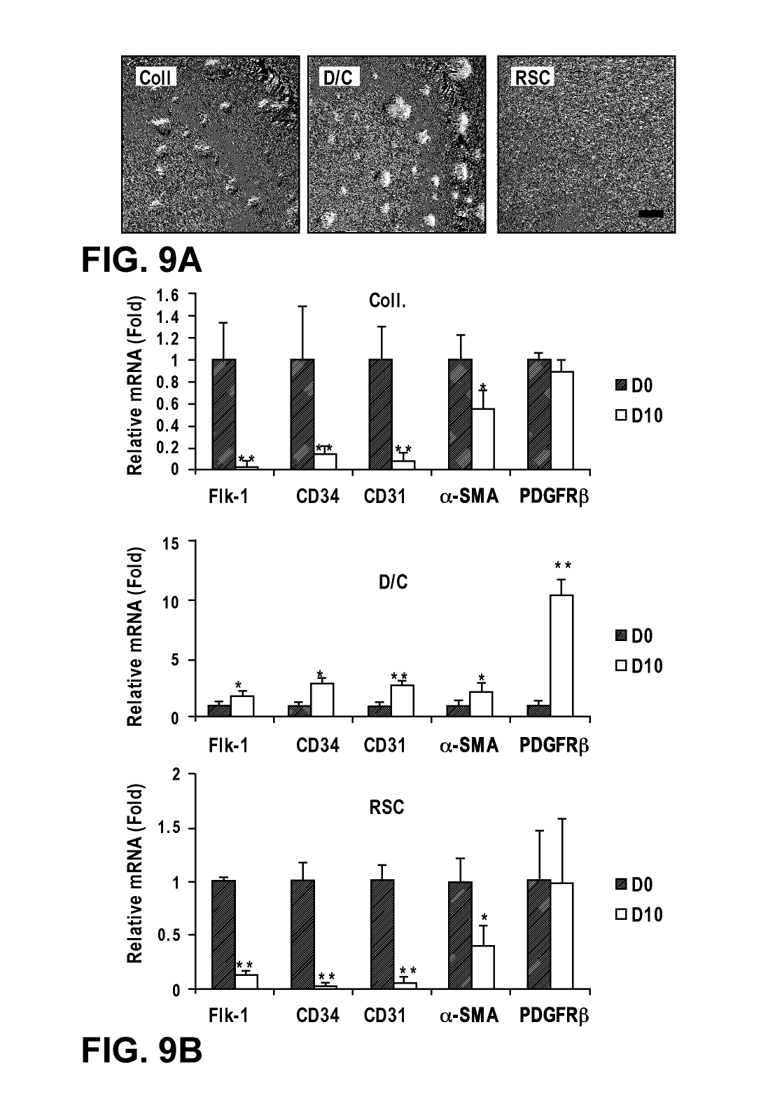

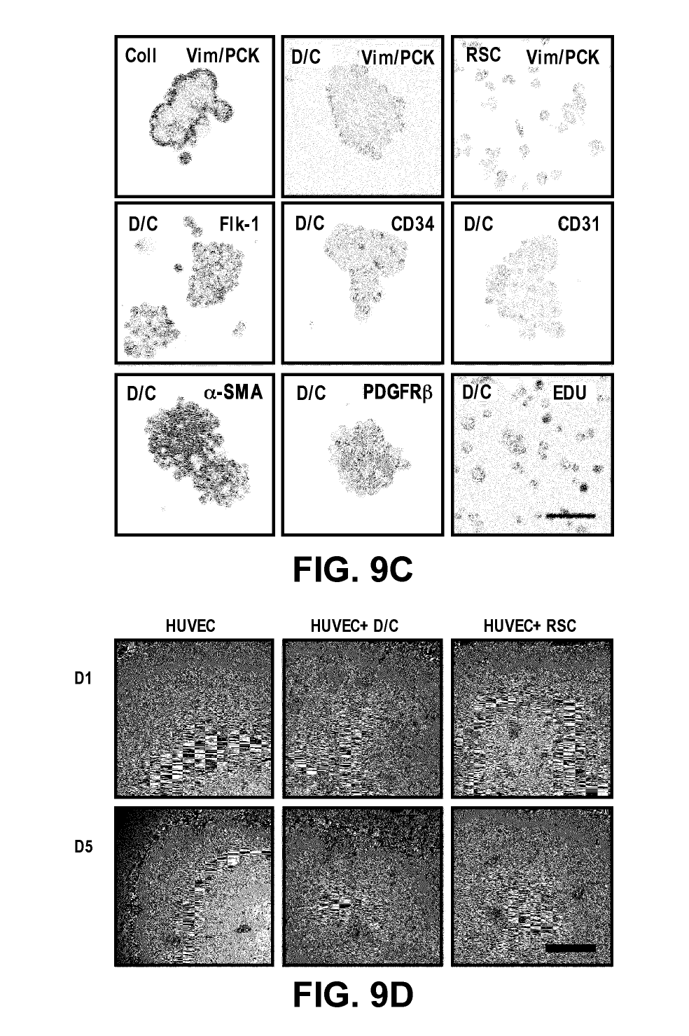

Collagenase-isolated limbal NC expanded on coated Matrigel turn into angiogenesis progenitor cells when reseeded in 3D Matrigel in MESCM. To determine whether D/C and RSC cells, of which both expressed angiogenesis markers in vivo (FIG. 8), could have the potential of differentiating into angiogenesis progenitors, we seeded them directly in 3D Matrigel immediately after isolation in MESCM. Single cells from collagenase-isolated clusters generate sphere growth during 10 days of culturing in ESCM. Herein, we noted that they also formed spheres during 10 days of culturing in MESCM (FIG. 9A). As a comparison, single cells from D/C clusters also generated spheres, but single RSC cells did not (FIG. 9A). When compared to cells immediately isolated at Day 0 (D0), spheres formed by collagenase-isolated cells at Day 10 expressed significantly less Flk-1, CD34, CD31, and α-SMA transcripts (FIG. 9B, P<0.05, n=3). A similar expression level was noted in single RSC cells cultured at Day 10. In contrast, expression levels of the aforementioned markers and that of PDGFRβ transcript were significantly upregulated in spheres formed by D/C isolated cells (FIG. 9B, P<0.05, n=3). Spheres formed by collagenase-isolated cells consisted of predominantly PCK+ epithelial cells and few Vim+ cells (FIG. 9C). Nonetheless, cells in D/C spheres and single RSC cells were exclusively Vim+(FIG. 9C), suggesting that Vim+ cells could be enriched in D/C clusters by culturing in 3D Matrigel. Immunostaining confirmed that Vim+ cells in D10 D/C spheres in 3D Matrigel expressed Flk-1, CD34, CD31, α-SMA, and PDGFR13 (FIG. 9C), but not SMMHC, which is a marker of smooth muscle cells, and not S100A4, which is a marker of myofibroblasts. These findings suggested that D10 D/C spheres in 3D Matrigel consisted of angiogenesis progenitors. The notion that these angiogenesis progenitors could serve as pericytes was confirmed by 5-day co-culturing with HUVEC on the surface of 100% Matrigel. Single cells from D10 D/C spheres could, but single RSC cells could not, stabilize the vascular network formed by HUVEC (FIG. 9D).

### Cells Expanded by Serial Passage on Coated Matrigel Express Both Pericyte and MSC Markers

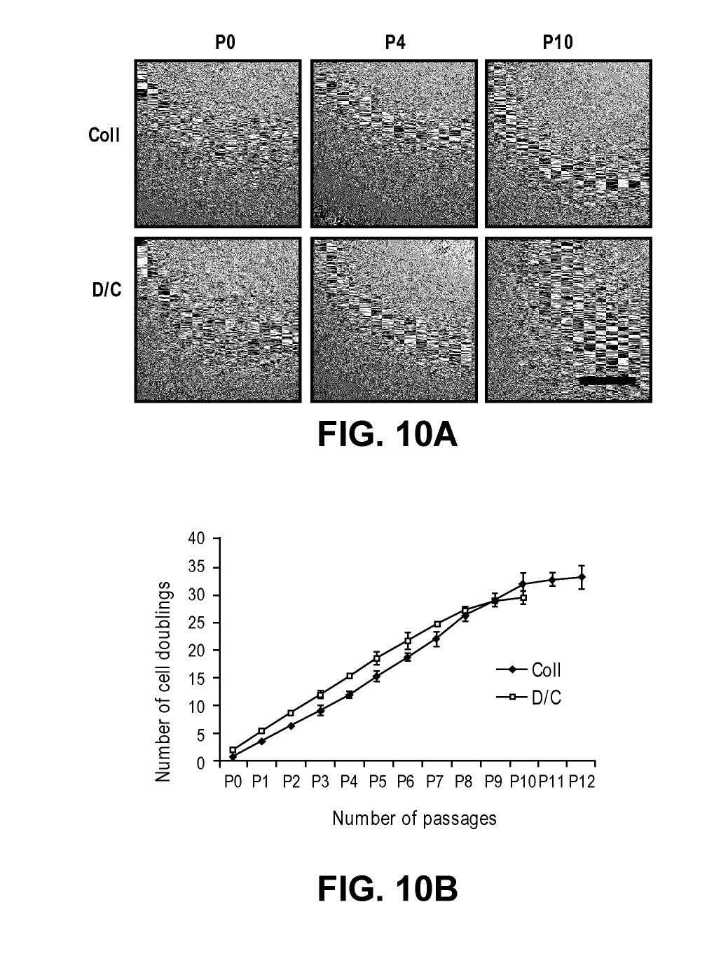

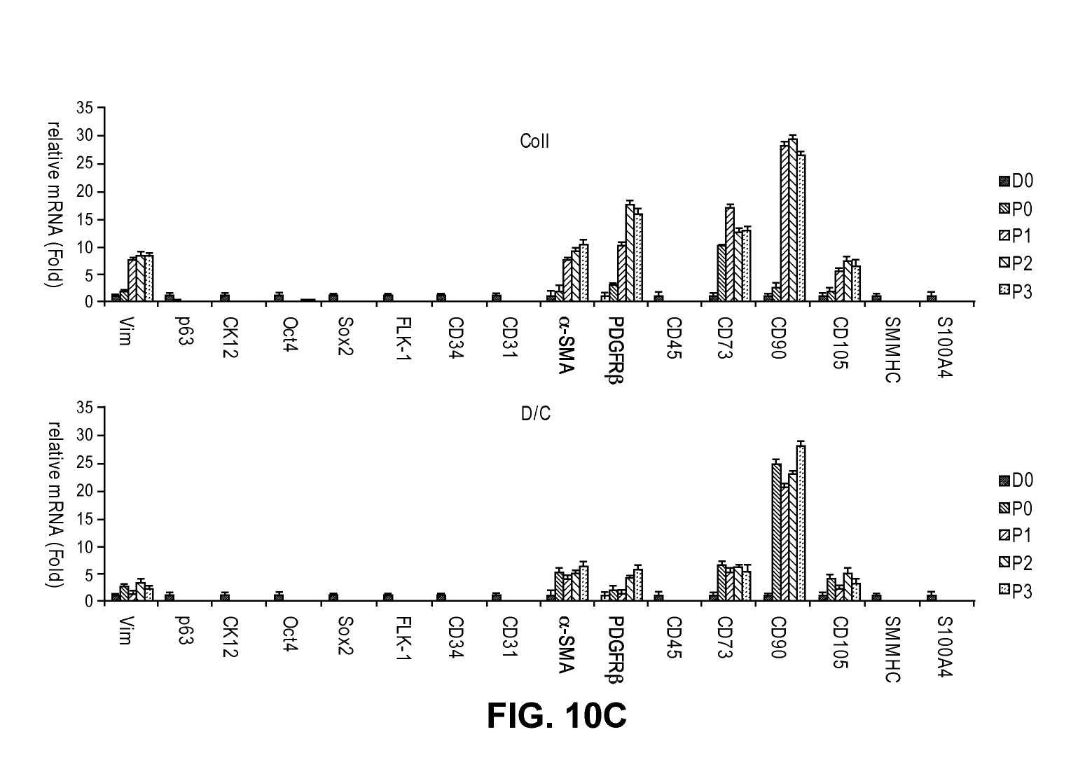

Cells from collagenase-isolated clusters exhibited poor proliferation if seeded in 3D Matrigel immediately after isolation. Herein, we also note that cells from D/C-isolated clusters exhibited poor proliferation as evidenced by low (5%) labeling by EdU, a thymidine analogue, when seeded immediately in 3D Matrigel to generate spheres (FIG. 9C, yellow merged nuclear fluorescence). To circumvent this limitation, collagenase-isolated cells can be expanded by a total of 12 passages if seeded on coated Matrigel in MESCM, resulting in 33 cell doublings and 1×10¹⁰ cells. Herein, we also found that D/C-isolated cells could similarly be expanded to yield spindle cells (FIG. 10A) and a growth potential for more than 10 passages (FIG. 10B). Similar to what we have reported for collagenase-isolated cells, compared to the expression level by D0 D/C-isolated cells, RT-qPCR revealed rapid extinction of p63 and CK12 transcripts during serial passages to P3 (FIG. 10C), indicating successful elimination of epithelial cells. Also similar to collagenase-isolated cells, expanded spindle cells from D/C-isolated cells also lost the expression of such ESC markers as Oct4 and Sox2 and such markers for endothelial progenitor cells as Flk-1, CD34, and CD31. Also similar to collagenase-isolated cells, the expression levels of Vim, α-SMA, and PDGFRβ transcripts were upregulated by an average of 2.5, 6.4, and 6 folds, respectively (FIG. 10C). Expanded spindle cells from both collagenase- and D/C-isolated cells did not express CD45 but upregulated expression of such MSC markers as CD73, CD90, and CD105 by an average of 5.8, 28, and 3.5 folds, respectively (FIG. 10C, n=3, P<0.05). They did not express SMMHC and S100A4 transcript, suggesting that they were neither smooth muscle cells nor myofibroblasts. Taken together, the above data suggested that limbal stromal cells isolated by the D/C method could be expanded on coated Matrigel in MESCM in a similar manner to those isolated by collagenase and that both expanded cells exhibited a similar growth potential and adopted a similar phenotype with features of pericytes and MSC.

### Phenotypic Change by Serial Passages on Plastic in DMEM/FBS

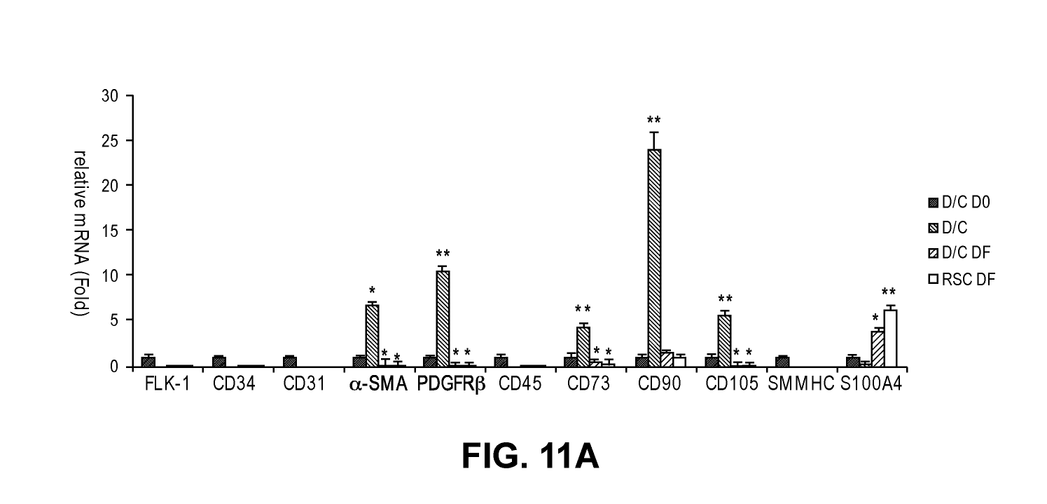

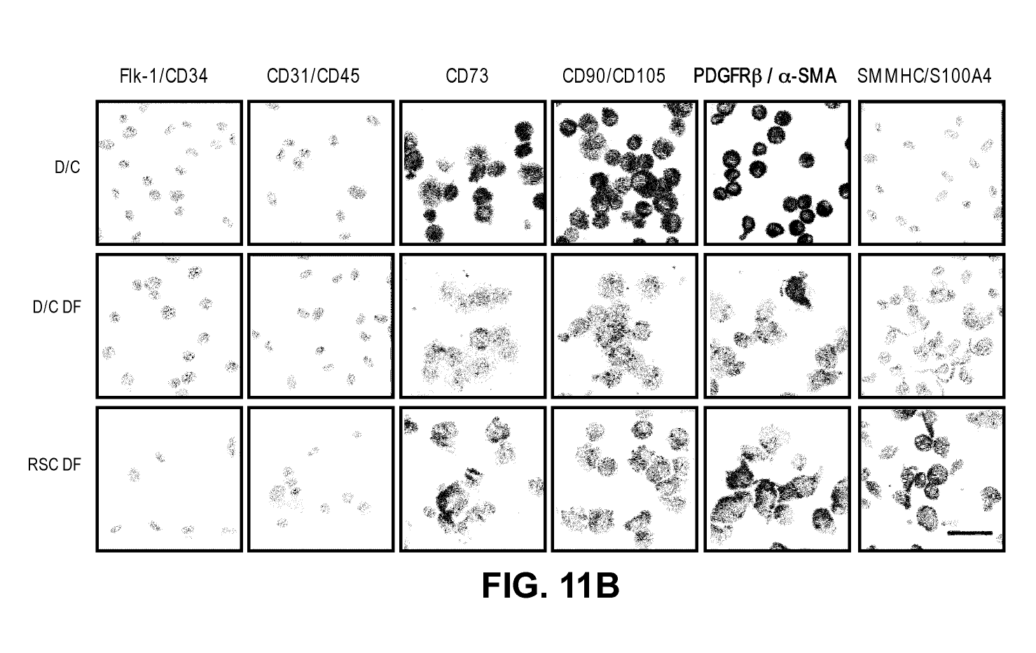

We then determined whether both D/C cells and RSC could also generate MSC by serial passages on plastic in DF, which is the conventional method of generating MSC. Similar to D/C cells expanded on coated Matrigel up to P3 (FIG. 10), D/C cells at P4 did not express Flk-1, CD34, CD31, and CD45 (FIG. 11A). The same result was noted for D/C cells and RSC expanded on plastic in DF at P4. Also similar to D/C cells cultured up to P3 (FIG. 10), D/C cells at P4 still upregulated expression of CD73, CD90, CD105, α-SMA, and PDGFRβ transcripts by 4.3, 24.0, 5.6, 6.8, and 10.5 folds, respectively (FIG. 11A, P<0.05 for CD73, but P<0.01 for all others, n=3). In contrast, except for CD90, of which a comparable level was expressed, significant downregulation of CD73, CD105, α-SMA, and PDGFRβ transcripts was noted in both D/C DF and RSC DF cells at P4 (FIG. 11A, P<0.05, n=3). As noted in FIG. 10, D/C cells expanded on coated Matrigel at P4 still did not express SMMHC and S100A4 transcripts. Although D/C DF and RSC DF cells at P4 did not express SMMHC transcript, both significantly upregulated expression of the S100A4 transcript, with RSC DF cells being more than D/C DF cells (FIG. 11A, P<0.05, n=3). The above expression pattern of different markers by D/C, D/C DF, and RSC DF cells was confirmed by immunostaining (FIG. 11B). Unlike D/C cells expanded on coated Matrigel, D/C DF cells lost expression of CD105 and PDGFRβ. RSC DF exhibited a similar phenotype to D/C DF cells except that they expressed more S100A4. These data indicate that both D/C cells and RSC expressed MSC markers but lost the pericyte phenotype while RSC adopted the myofibroblast phenotype when they were expanded on plastic in DF.

### Higher CFU-F and Tri-Lineage Differentiation by Cells Expanded on Coated Matrigel

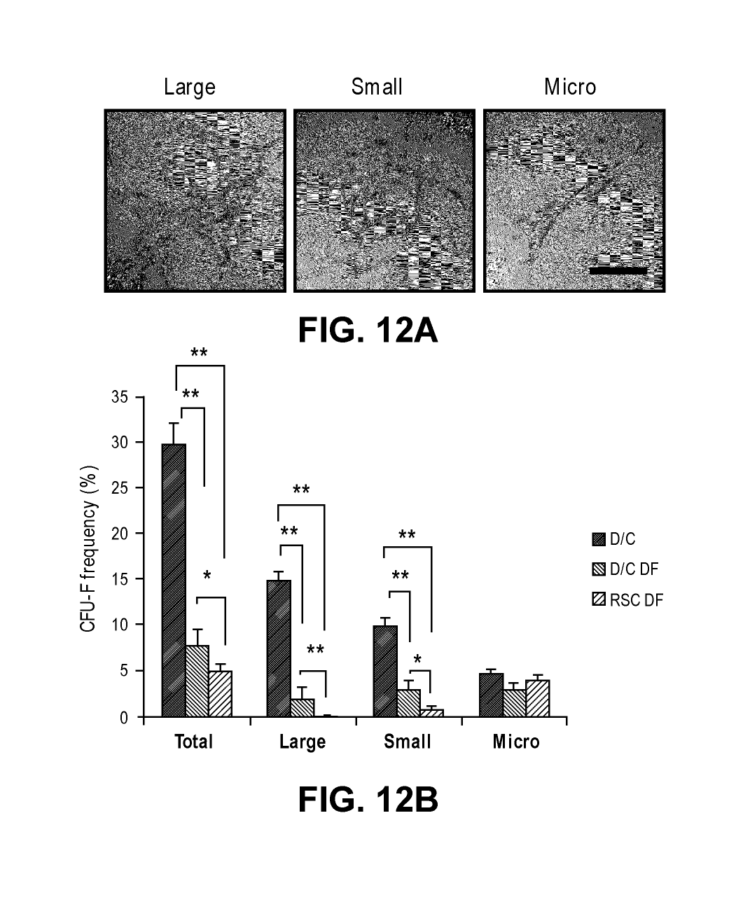

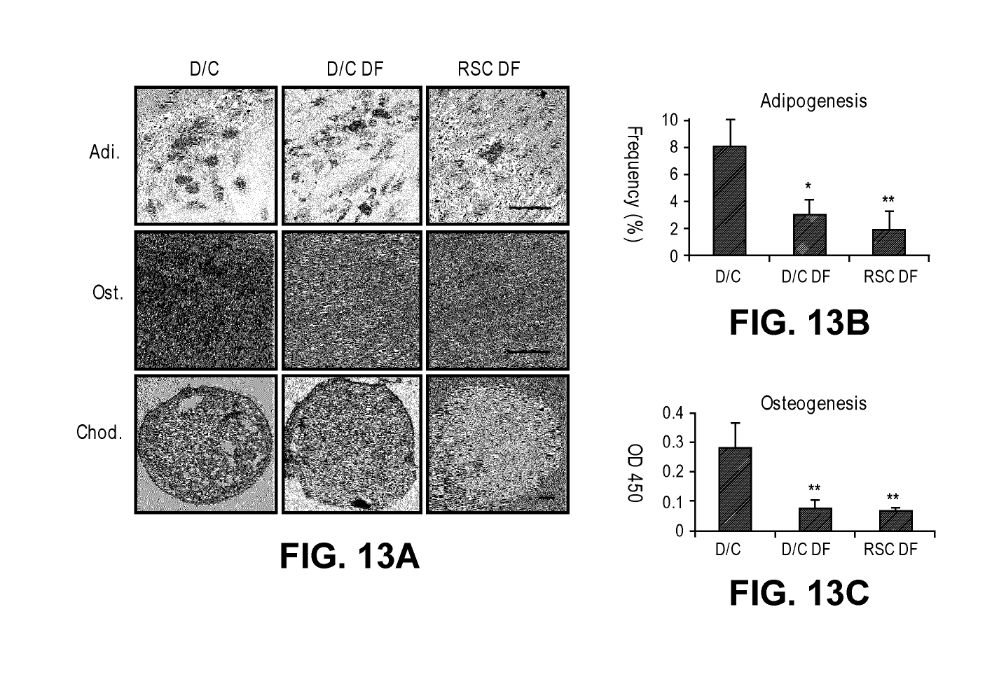

To further demonstrate that above cells expressing MSC markers phenotype were indeed MSC, we compared CFU-F, an in vitro way of evaluating MSC function, and differentiation into osteogenic, chondrogenic, and adipogenic lineages. Our results showed that D/C cells expanded on coated Matrigel in MESCM at P4 (D/C) exhibited the highest CFU-F, judged by either a total or by three different clones when compared to D/C DF or RSC DF cells expanded at P4 (FIG. 12B, P<0.05, n=3). The CFU-F of D/C DF cells was significantly higher than that of RSC DF cells (FIG. 12B, P<0.05, n=3). When these three cells were cultured in the medium designated for adipogenesis, osteogenesis, and chondrogenesis, respectively, we noted that although they all could differentiate into adipocytes, osteocytes, and chondrocytes (FIG. 13A), D/C cells were significantly more potent than D/C DF and RSC DF cells (FIGS. 13B and 13C). There was no difference in adipogenesis and osteogenesis between D/C DF and RSC DF cells, but D/C DF cells were more potent than RSC DF cells in chondrogenesis.

### Corneal Differentiation and Stemness of LEPC are Affected by Different Mesenchymal Cells

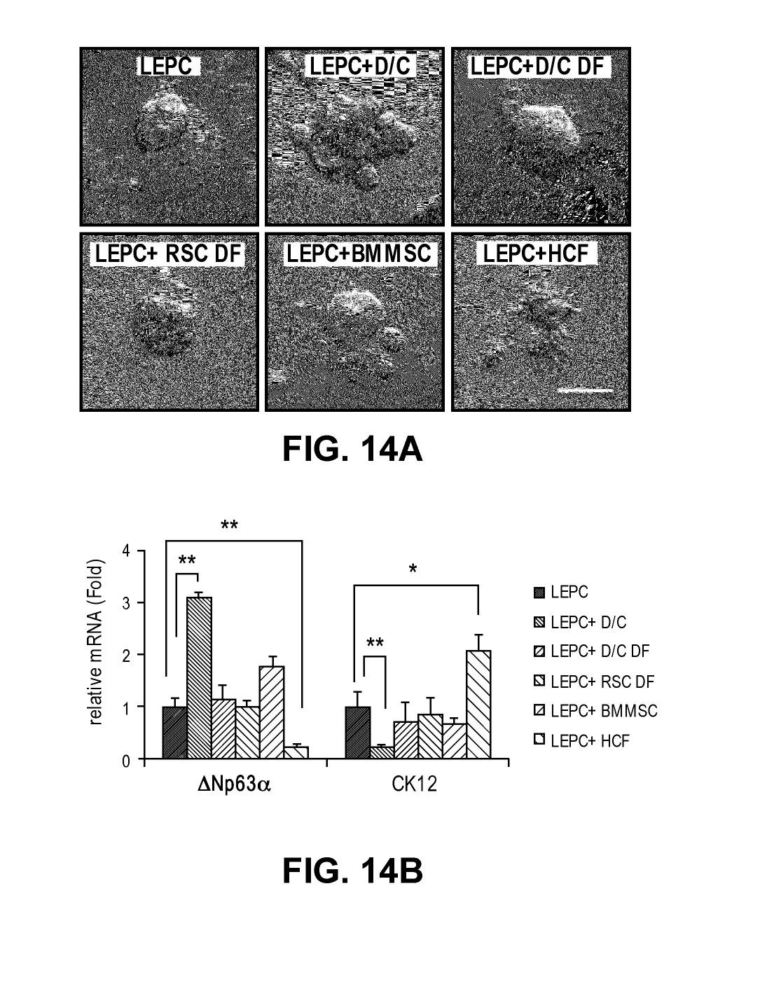

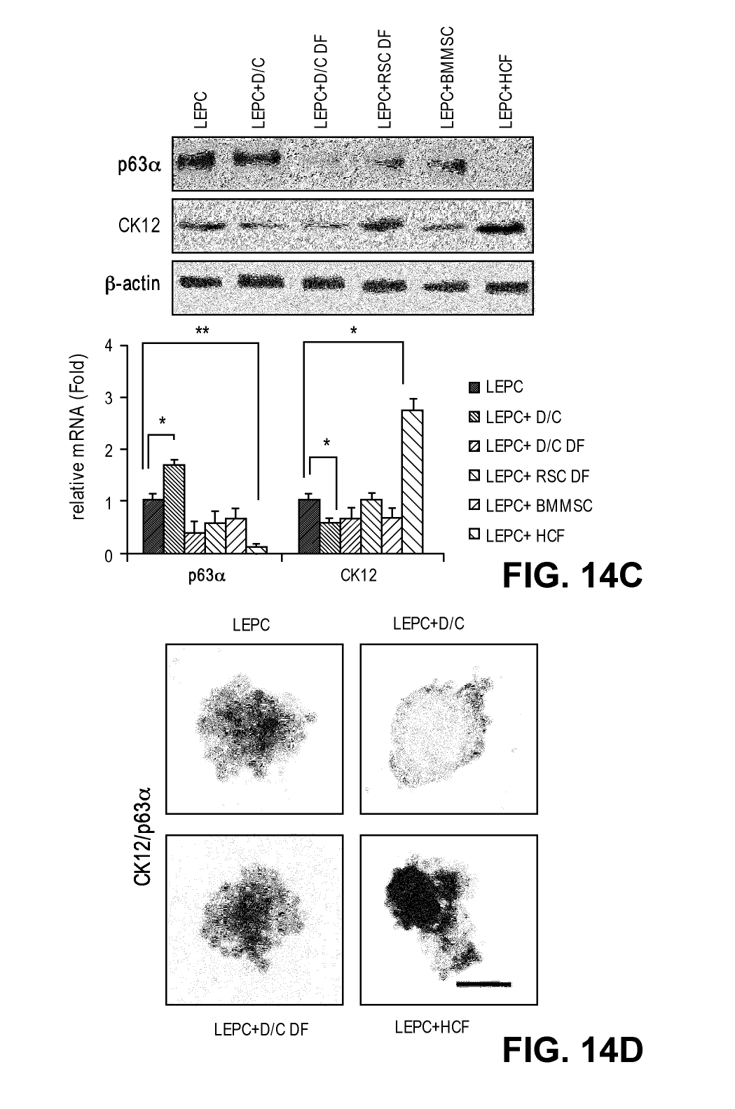

Collagenase-isolated cells expanded on coated Matrigel in MESCM prevent corneal epithelial differentiation of dispase-isolated LEPC judged by expression of CK12 when both single cells were recombined to form spheres in 3D Matrigel. To determine whether similarly expanded D/C cells could also serve as NC to support LEPC, we performed the same assay and compared to D/C DF and RSC DF cells, all expanded up to P4. We also compared to BMMSC and HCF cells that had been cultured on plastic in DF. All these mesenchymal cells could quickly form reunion with LEPC to yield spheres in 10 days of culturing in 3D Matrigel (FIG. 14A). Compared to that by spheres formed by LEPC alone, expression levels of the ΔNp63α transcript was significantly upregulated in LEPC+D/C spheres and LEPC+BMMSC spheres (to a lesser extent), but significantly downregulated in LEPC+HCF spheres (FIG. 14B, *P<0.05, **P<0.01, n=3). In contrast, expression of the CK12 transcript was significantly downregulated in LEPC+D/C spheres but significantly upregulated in LEPC+HCF spheres, while not significantly changed in LEPC+D/C DF, LEPC+RSC DF, and LEPC+BMMSC spheres (FIG. 14B, *P<0.05, **P<0.01, n=3). The above transcript expression pattern was consistent with the protein level of p63α and CK12 using β-actin as a loading control in Western blots (FIG. 14C, P<0.01, n=3) and consistent with the extent of double immunostaining between CK12 and p63α (FIG. 14D). These results indicated that D/C-isolated limbal stromal cells expanded on coated Matrigel also served as NC to prevent corneal epithelial differentiation of LEPC more efficiently than their counterparts and RSC expanded on plastic in DF. As a contrast, HCF expanded on plastic in DF stimulated full-blown corneal epithelial differentiation with the loss of p63α expression.

### Example 7

### Isolation of Limbal Epithelial Sheets and Clusters

Human corneoscleral rims from donors younger than 60 years and less than 5 days from death to culturing were obtained from the Florida Lions Eye Bank and managed in accordance with the declaration of Helsinki. The isolation of limbal epithelial sheets or clusters by either dispase or collagenase, respectively, followed what we have reported. In short, after corneoscleral tissue was rinsed three times with HBSS containing 50 μg/ml gentamicin and 1.25 μg/ml amphotericin B, the remaining sclera, conjunctiva, iris, trabecular meshwork and corneal endothelium were removed. Then, the tissues were cut into 12 one-clock-hour segments, from which each limbal segment was obtained by incisions made at 1 mm within and beyond the anatomic limbus. An intact epithelial sheet was isolated by digesting each limbal segment at 4° C. for 16 h with 10 mg/ml dispase II in MESCM made of DMEM/F-12 (1:1) supplemented with 10% knockout serum, 5 μg/ml insulin, 5 μg/ml transferrin, 5 ng/ml sodium selenite, 4 ng/ml bFGF, 10 ng/ml hLIF, 50 μg/ml gentamicin, and 1.25 μg/ml amphotericin B. In parallel, other limbal segments were digested at 37° C. for 18 h with 1 mg/ml collagenase A in MESCM, SHEM or DF to generate limbal clusters. SHEM consists of DMEM/F-12 (1:1) supplemented with 5% fetal bovine serum (FBS), 0.5% dimethyl sulfoxide, 2 ng/ml hEGF, 5 μg/ml insulin, 5 μg/ml transferrin, 5 ng/ml selenium, 0.5 μg/ml hydrocortisone, 1 nM cholera toxin, 50 μg/ml gentamicin, and 1.25 μg/ml amphotericin B. DF is made of DMEM containing 10% FBS, 50 μg/ml gentamicin and 1.25 μg/ml amphotericin B. Limbal epithelial sheets and clusters were further digested with 0.25% trypsin and 1 mM EDTA (T/E) at 37° C. for 15 min to yield single cells.

### Coated, 2D and 3D Matrigel Culture and Treatment

Matrigel with different thicknesses, i.e., coated, thin (2D), and thick (3D) gel, were prepared by adding the plastic dish with 5% diluted Matrigel, 50 μl 50% diluted Matrigel, and 200 μl of 50% diluted Matrigel (all in DMEM) per cm², respectively, by incubation at 37° C. for 1 h before use. On 3D Matrigel, dispase and collagenase-isolated cells were seeded at the density of 5×10⁴ per cm² in MESCM. In parallel, on coated and 2D Matrigel, 5×10⁴ or 1×10⁵ collagenase-isolated cells per cm² were seeded in MESCM, SHEM or DF. At Passage 0 (P0), cells on Day 5 cultured in coated, 2D and 3D Matrigel in MESCM were added with 10 μM of 5-ethynyl-2′-deoxyuridine (EdU) for 24 h. Spheres in 3D gel at different time points were harvested by digestion in 10 mg/ml dispase II at 37° C. for 2 h, of which some were rendered into single cells by T/E. Upon 80% confluence on coated Matrigel, single cells were continuously passaged at the density of 5×10³ cells per cm². At P4, the expanded cells were also re-seeded in 3D Matrigel at the density of 5×10⁴ cells per cm² in three different medium for 6 days. Afterwards, P4 expanded cells from 3D Matrigel were pre-labeled with red fluorescent nanocrystals (Qtracker® cell labeling kits, Invitrogen), mixed at 1:4 ratio with dispase-isolated epithelial cells, and seeded at the density of 5×10⁴ per cm² in 3D Matrigel containing MESCM and cultured for 10 days. The extent of total expansion was measured by the number of population-doubling from P1 to P4 using the following formula: Number of Cell Doublings (NCD)=log₁₀(y/x)/log₁₀2, where “y” is the final density of the cells and ‘x’ is the initial seeding density of the cells.

### 3T3 Clonal Cultures

The epithelial progenitor status of the sphere growth was determined by a clonal assay on 3T3 fibroblast feeder layers in SHEM. The feeder layer was prepared by treating 80% subconfluent 3T3 fibroblasts with 4 μg/ml mitomycin C at 37° C. for 2 h in DMEM containing 10% newborn calf serum before being seeded at the density of 2×10⁴ cells per cm². Single cells obtained from Day 10 spheres were then seeded on mitomycin C-treated 3T3 feeder layers, at a density of 100 cells per cm² for 2 weeks. Resultant clonal growth was assessed by rhodamine B staining, and the colony-forming efficiency (CFE) was measured by calculating the percentage of the clone number divided by the total number cells seeded. The clone morphology was subdivided into holoclone, meroclone, and paraclone based on the criteria for skin keratinocytes.

### Immunofluorescence Staining

Limbal epithelial sheets or clusters obtained by dispase or collagenase digestion, respectively, were cryosectioned to 6 μm. Spheres, EdU labeled cells and the P4 isolated mesenchymal cells were prepared for cytospin using Cytofuge® at 1,000 rpm for 8 min. For immunoflorescence staining, 4% formaldehyde-fixed samples were permeated with 0.2% Triton X-100 in PBS for 15 min and blocked with 2% BSA in PBS for 1 h at room temperature before being incubated in the primary antibody overnight at 4° C. Corresponding secondary antibodies were then incubated for 1 h using appropriate isotype-matched non-specific IgG antibodies as controls. EdU-labeled cells were detected by fixation in 4% formaldehyde for 15 min followed by 0.2% Triton X-100 in PBS for 15 min, blocking with 2% BSA in PBS for 1 h, and incubation in Click-iT™ reaction cocktails (Invitrogen) for 30 min before subjecting to PCK immunostaining Nuclear counterstaining was achieved by Hoechst 33342 before being analyzed with a Zeiss LSM 700 confocal microscope.

Quantitative Real-Time Polymerase Chain Reaction (qRT-PCR)

Total RNAs were extracted from limbal clusters freshly isolated by collagenase on Day 0, cells on coated and 3D gel at different passages by RNeasy Mini RNA isolation kit. A total of 1-2 μg of total RNAs was reverse-transcribed to cDNA by high capacity cDNA transcription kit. qRT-PCR was carried out in a 20 μl solution containing cDNA, TaqMan Gene Expression Assay and universal PCR master Mix. The results were normalized by internal control, glceraldehyde-3-phosphate dehydrogenase (GAPDH). The relative gene expression data was analyzed by the comparative C_{T} method (ΔΔC_{T}).

### Immunoblot Analysis

Proteins from Day 10 spheres were extracted by RIPA buffer supplemented with proteinase inhibitors and phosphatase. The protein concentration was determined by a BCA protein assay. Equal amounts of proteins in total cell extracts were separated by 10% SDS-PAGE and transferred to nitrocellulose membranes which were then blocked with 5% (w/v) fat-free milk in TBST (50 mM Tris-HCl, pH 7.5, 150 mM NaCl, 0.05% (v/v) Tween-20) followed by sequential incubation with specific primary antibodies and their respective secondary antibodies using β-actin as the loading control. The immunoreactive bands were visualized by a chemiluminescence reagent.

### Results

### Collagenase Isolates More Subjacent Mesenchymal Cells

Digestion with dispase removed an intact human limbal epithelial sheet (FIG. 15A) that consisted nearly exclusive PCK+ cells (FIG. 15B). Nonetheless, digestion with collagenase resulted in a cluster of cells (FIG. 15C) that consisted of not only entire PCK+ epithelial cells but also many subjacent PCK−/Vim+ cells (FIG. 15D). These results indicated that collagenase, but not dispase, could isolate both limbal progenitors/ and closely associated stromal MCs.

### Clusters Isolated by Collagenase in MESCM Express Most ESC Markers

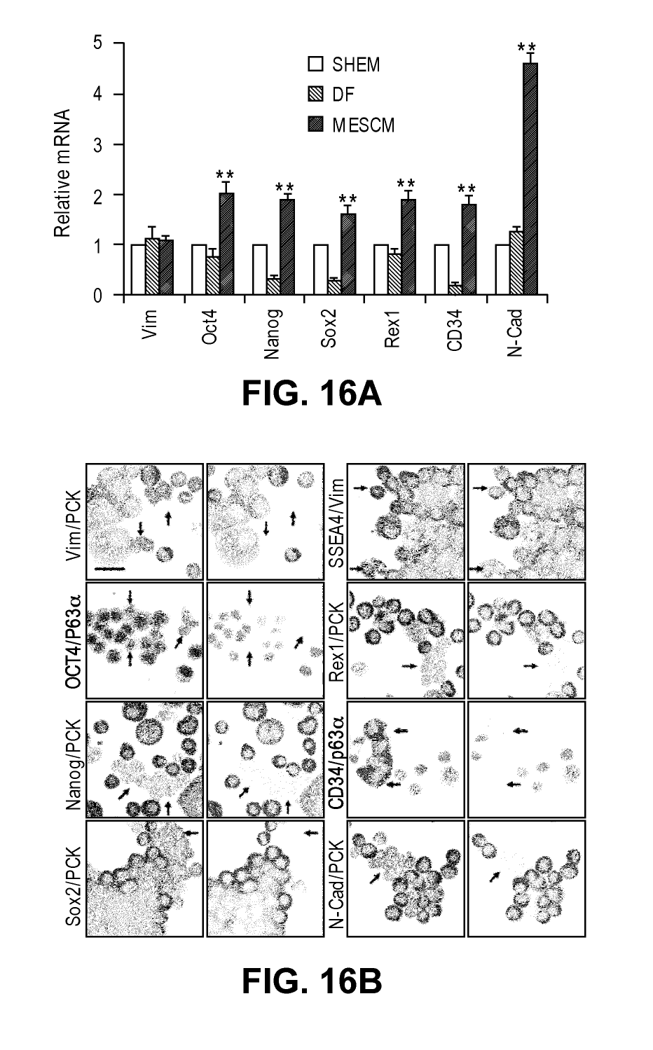

We isolated the aforementioned limbal clusters by collagenase digestion in SHEM, which contains FBS. MCs in such collagenase-isolated limbal clusters are as small as 5 μm in diameter and heterogeneously express various SC markers including Oct4, Sox2, Nanog, Rex1, SSEA4, Nestin, N-Cadherin, and CD34. To prepare further isolation of these putative NCs, we digested limbal segments with collagenase in MESCM and compare the expression of the above markers to that in SHEM or DF. qRT-PCR showed that the transcript level of Vim was not different among these three medium (FIG. 16A), suggesting that they resulted in similar numbers of MCs. However, the transcript levels of Oct4, Nanog, Sox2, Rex1, CD34, and N-Cadherin in MESCM were all significantly higher than those in SHEM and DF (n=3, all P<0.01, FIG. 16A), suggesting that expression of these markers by collagenase-isolated clusters was better maintained in MESCM. As a comparison, except for that of Oct4, Rex1 and N-Cadherin, expression of all other markers was markedly reduced in DF (FIG. 16A). Our previous study showed that all small PCK+ epithelial cells were p63α+ but Vim−. Thus, we performed double immunostaining in PCK−, p63α−, or Vim+ MCs with the above SC markers. The results showed that these small non-epithelial MCs indeed heterogeneously expressed Oct4, Nanog, SSEA4, Sox2, Rex1, CD34, and N-Cadherin (FIG. 16B). Some of these SC markers were also expressed in some small epithelial cells. Collectively, these findings indicated that expression of these SC markers by small PCK−/p63α−/Vim+ cells was best maintained during collagenase digestion in MESCM.

### Different Growths on Three Matrigel Substrates

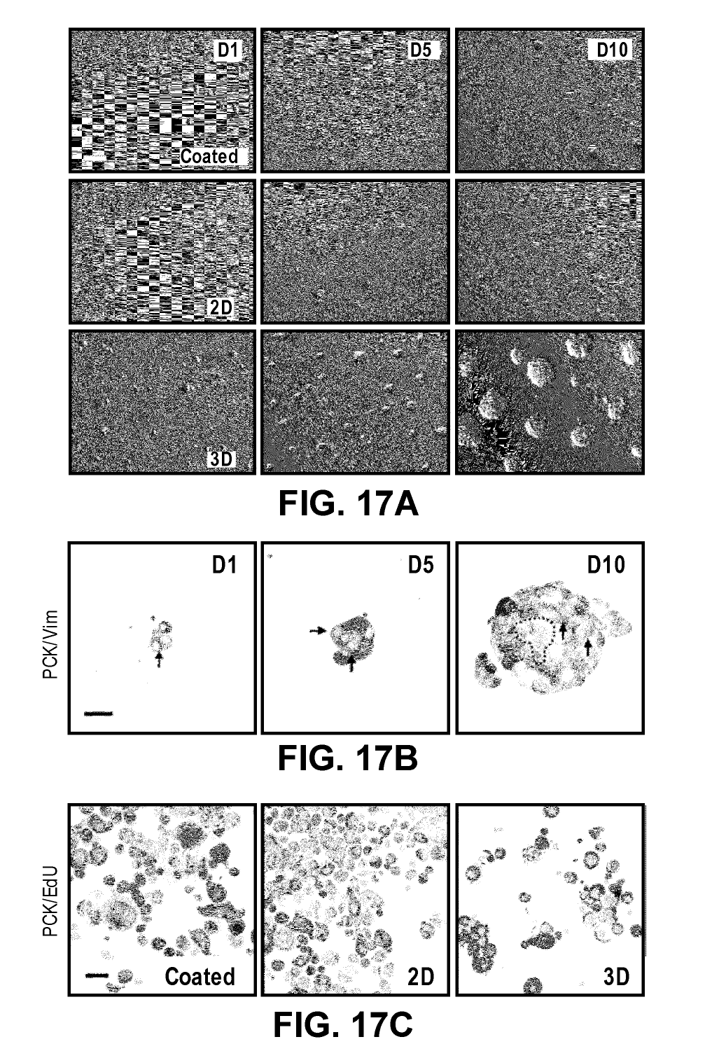

Our previous study showed that disruption of close association between PCK+ epithelial progenitors and Vim+MCs diminishes epithelial clonal growth in three different assays, suggesting that the latter might serve as NCs. We speculated that such close association between PCK+ and Vim+ cells in collagenase-isolated clusters might be attained by preservation of the basement membrane. We then reasoned that single PCK+ and Vim+ cells generated by T/E, which disrupted their close association in collagenase-isolated clusters, might be reunited in the basement membrane substrate prepared by Matrigel. Indeed, spheres emerged in 3D Matrigel when cultured in MESCM, while predominant spindle cells without spheres occurred in coated and 2D Matrigel (FIG. 17A). Double immunostaining showed that spheres formed in 3D Matrigel consisted of both PCK+ cells and Vim+ cells on Day 1 and both cells increased in number on Day 5 and Day 10 (FIG. 17B). The proliferative activity measured by nuclear EdU labeling on Day 5 for 24 h was higher in coated and 2D Matrigel than 3D Matrigel (FIG. 17C). The labeling index was 25.6±3.2% and 27.3±2.6% in PCK+ cells, and 13.6±1.5% and 12.9±2.4% in PCK− cells in coated and 2D Matrigel, respectively. They were significantly higher than 12.5±2.0% in PCK+ cells and 2.6±1.2% in PCK− cells in 3D Matrigel (n=5, all P<0.01). These results suggested that cell proliferation was higher on coated and 2D Matrigel, where spindle cells emerged, than in 3D Matrigel, where sphere growth formed by reunion of single PCK+ and Vim+ cells.

Spindle Cells Proliferate and Dominate on Coated Matrigel after Serial Passages

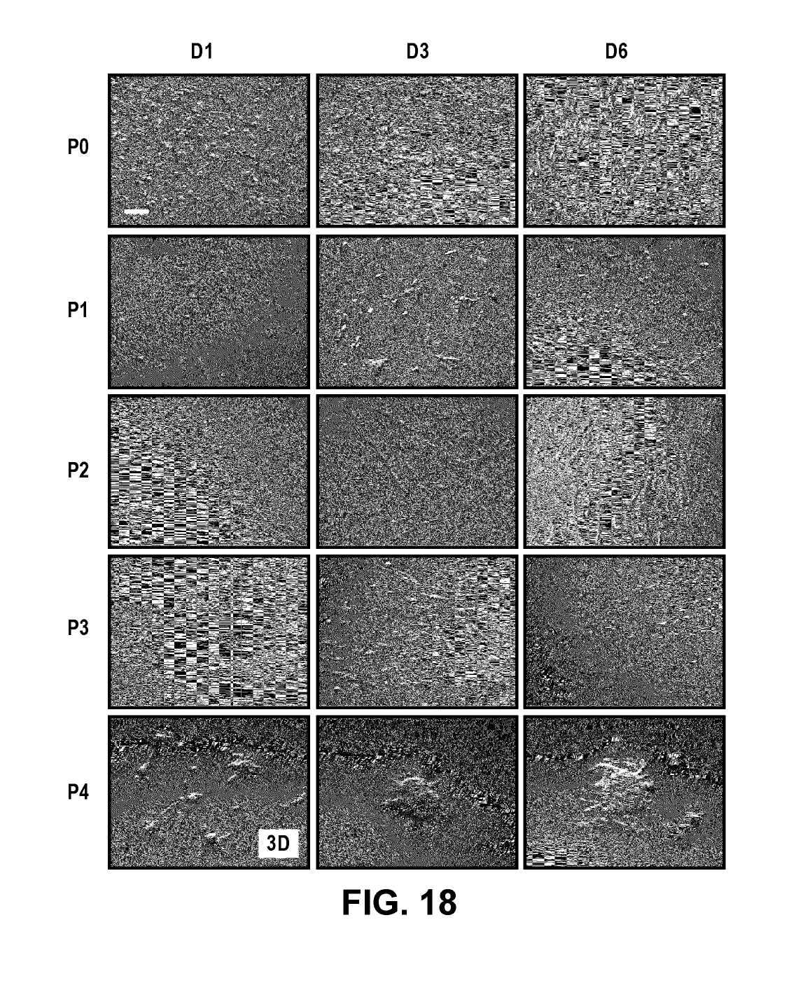

Because spheres formed in 3D Matrigel contained both PCK+ and Vim+ cells and Vim+ cells therein grew slower than PCK+ cells when judged by the EdU labeling index (FIG. 17), 3D Matrigel was not an ideal substrate for isolating and expanding Vim+ MCs. In contrast, spindle cells emerged among small round cells on coated Matrigel, and rapidly increased in number upon further passages (FIG. 18). Although some small round cells were noted in P0, spindle cells dominated from P2 onward (FIG. 18). When re-seeded in 3D Matrigel, single P 4 cells began to form aggregates with stellate borders as early as Day 1, increased in size, but ceased to grow on Day 6 (FIG. 18). The above changes of the proliferative activity were also reflected by the population doubling time, which was 40 and 39 h for spindle cells at P2 and P3 in coated Matrigel but was significantly lengthened to 881 h when re-seeded in 3D Matrigel at P4 (Table 12).

TABLE 12
Population Doubling of Cultured Mesenchymal Cells from Collagenase-Isolated
Clusters.

| | | | | Number | | |
|---|---|---|---|---|---|---|
| | Seeding | Culture | Final | of Cell | | Population |
| | Density | Time | density | Doublings | Accumulative | Doubling |
| Passage | (×10⁵/cm²) | (days) | (×10⁵/cm²) | (NCD) | NCD | Time (h) |
| P0 | 1 | 6 | 1.05 | 0.07 | 0.07 | 2045.8 |
| P1 | 0.05 | 6 | 0.09 | 0.85 | 0.92 | 169.8 |
| P2 | 0.05 | 6 | 0.6 | 3.58 | 4.50 | 40.2 |
| P3 | 0.05 | 6 | 0.66 | 3.72 | 8.23 | 38.7 |
| P4 | 0.5 | 6 | 0.56 | 0.16 | 8.39 | 880.7 |

### Reversibility of Phenotype of Spindle Cells Expanded in MESCM

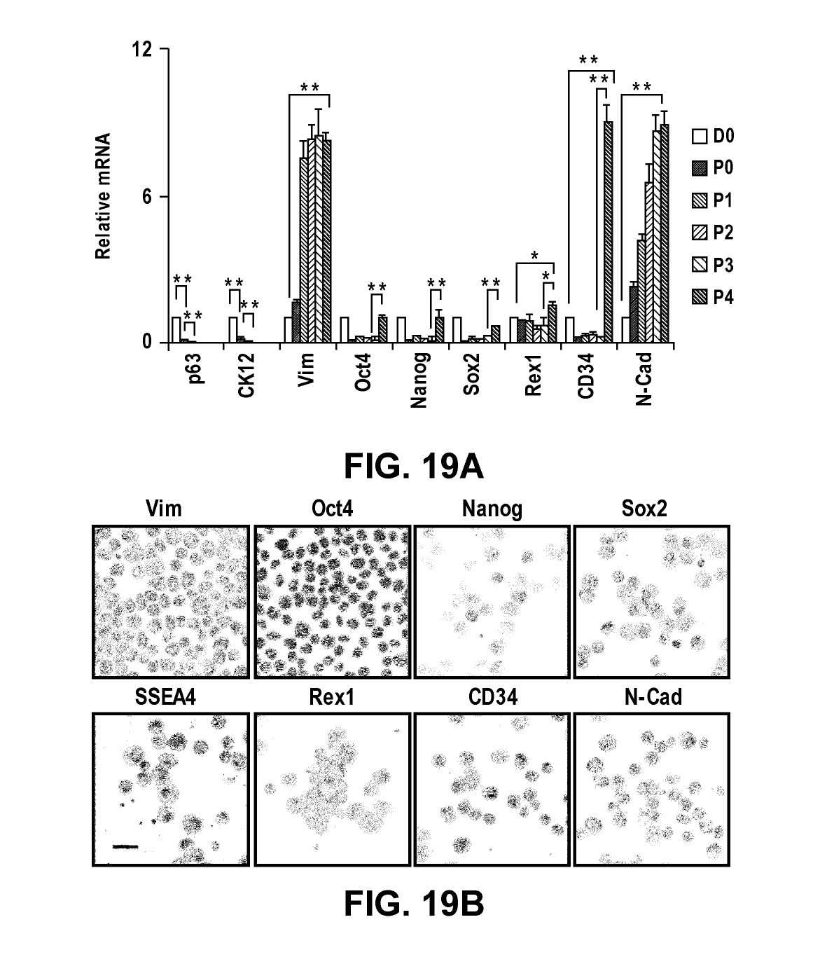

Compared to that of the D0 cluster immediately isolated by collagenase, qRT-PCR revealed a rapid disappearance of p63, i.e., an epithelial progenitor marker¹³, and CK12, i.e., a corneal epithelial differentiation marker by P2 cells (FIG. 19A), suggesting that coated Matrigel successfully eliminated all epithelial cells by successive passages. From P0 to P3, there was a significant decline in expression of Oct4, Nanog, Sox2 and CD34 transcripts but a steady significant increase of expression of Vim and N-cadherin transcripts (FIG. 19A, all P<0.01, n=3). Upon being re-seeded in 3D Matrigel at P4, the transcript levels of Oct4, Nanog, Sox2 Rex1, and CD34 were significantly increased when compared to P3 cells (all P<0.01, n=3), while those of CD34, Rex1, and N-cadherin were significantly higher than that of D0 clusters (all P<0.01, n=3). When reseeded in 3D Matrigel, these spindle cells at P4 indeed re-expressed Oct4, Nanog, SSEA4, Sox2, Rex1, CD34, and N-Cadherin (FIG. 19B).

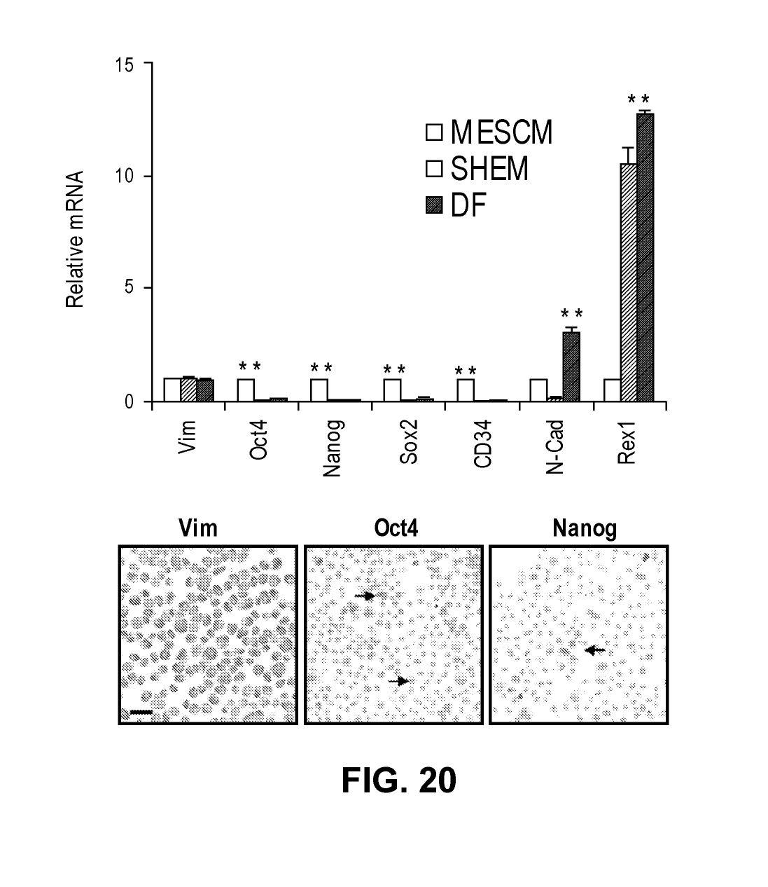

As a comparison, we also isolated and expanded spindle MCs on coated Matrigel in SHEM and DF. Upon being reseeded in 3D Matrigel at P4, they also formed similar aggregates. However, qRT-PCR showed that these cells did not regain expression of these SC markers (FIG. 20A). Immunostaining confirmed the lack of such expression (FIG. 20B). Collectively, these data showed that the phenotype of expressing embryonic SC markers was regained by spindle cells expanded via continuous passages on coated Matrigel only in MESCM but not in SHEM or DF.

### Spheres Formed by Reunion Between Dispase-Isolated Epithelial Cells and MCs Isolated and Expanded in Different Medium

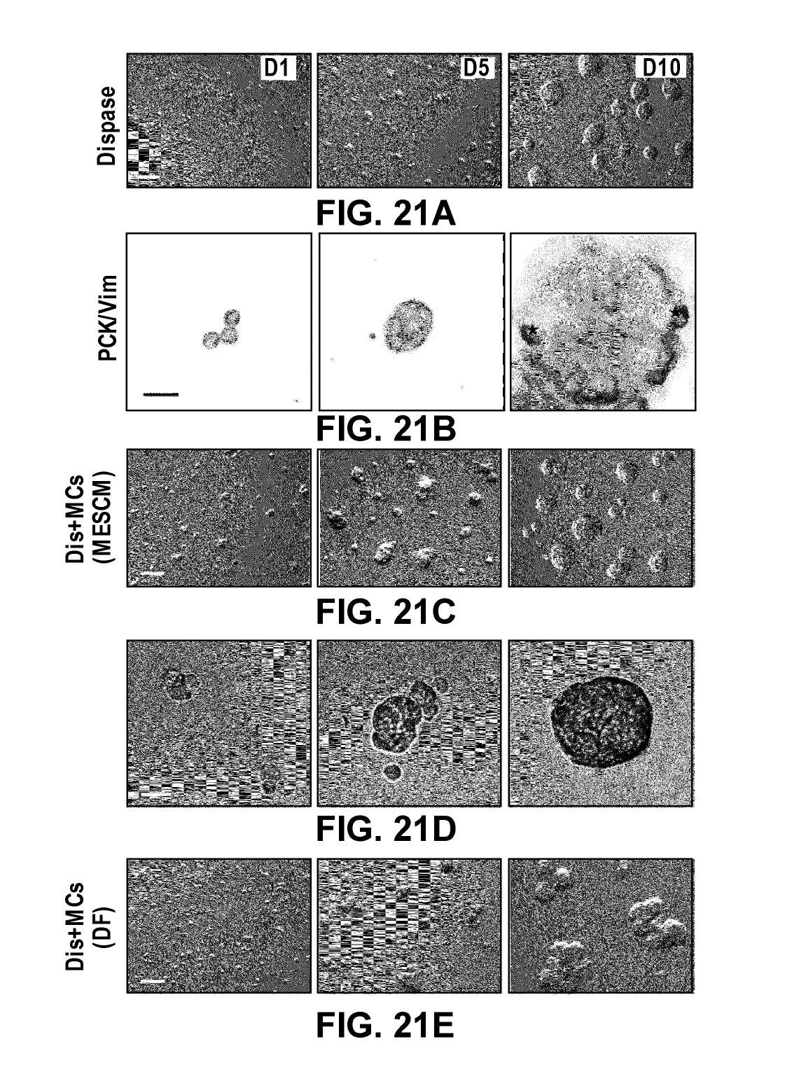

FIG. 3 showed that reunion between PCK+ epithelial cells and Vim+ MCs obtained from collagenase-isolated clusters led to sphere growth. We found out that PCK+ epithelial cells obtained from dispase-isolated limbal epithelial sheets, which contained few Vim+ cells (FIG. 15B), could also yield similar sphere growth in 3D Matrigel containing MESCM (FIG. 21A). Double immunostaining shows that these spheres consisted of predominantly PCK+ epithelial cells of which few also co-expressed Vim on Day 10 (FIG. 21B). Thus, we mixed dispase-isolated epithelial cells with MCs that had been expanded on coated Matrigel up to P4 followed by seeding in 3D Matrigel in different medium at the ratio of 4:1 to match with the finding that 20% of collagenase-isolated clusters is made of PCK−/Vim+MCs. Spheres generated by MCs expanded in MESCM were relatively more and larger (FIGS. 21A and 21C). These spheres consisted of epithelial cells and MCs pre-labeled by red Qdot®nanocrystals (FIG. 21D). In contrast, spheres generated by mixing with MCs expanded in DF tended to adhere to one another on Day 10 (FIG. 21E), which also consisted of both epithelial cells and MCs pre-labeled by red Qdot®nanocrystals.

### Maintenance of Limbal Epithelial Progenitor Status by Expanded NCs

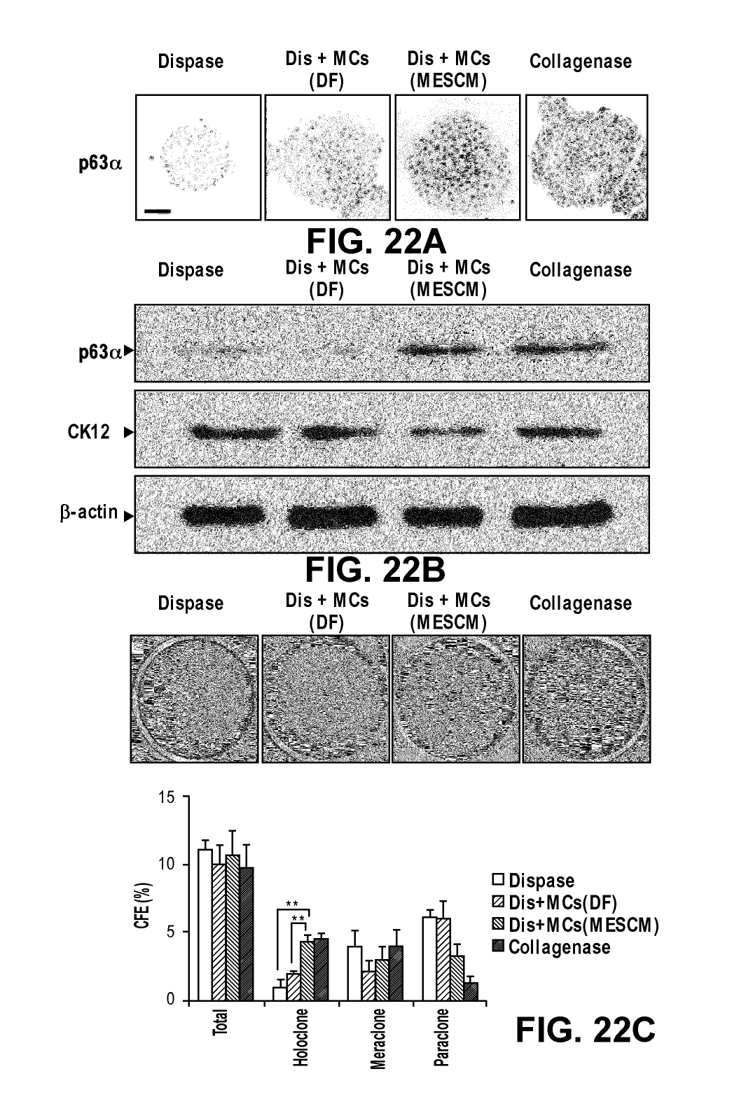

Although similar spheres were formed by dispase-isolated epithelial cells with or without being mixed with expanded MCs (FIG. 21), immunofluorescence staining of p63α showed that Dispase+MCs (MESCM) had more p63α expression than Dispase+MCs (DF) (FIG. 22A). Western blot analysis followed by densitometry confirmed that spheres formed by collagenase-isolated limbal clusters (FIG. 17) expressed 3.5 fold p63α and 0.6 fold CK12 when compared to those formed by dispase-isolated limbal epithelial sheets (FIG. 22B). Compared to spheres formed by dispase-isolated limbal epithelial cells, addition of MCs expanded in MESCM resulted in spheres expressing 3.9 fold more p63α and 0.5 fold less CK12, i.e., to a level similar to those formed by collagenase-isolated clusters. In contrast, addition of MCs expanded in DF resulted in spheres expressing 0.7 fold p63α and 0.7 fold CK12 (FIG. 22B). Spheres from collagenase-isolated limbal clusters generated more holoclones than dispase-isolated limbal epithelial sheets on growth-arrested 3T3 feeder layers presumably because of inclusion of the entire limbal basal epithelial progenitors (FIG. 22C). Compared to this baseline finding, spheres generated by mixing dispase-isolated epithelial cells with MCs expanded in MESCM had a significantly more holoclone than those mixed with MCs isolated in DF (FIG. 22C). Collectively, these findings suggested that reunion with MCs expanded in MESCM prevent corneal epithelial differentiation and promoted clonal growth of limbal epithelial progenitors similar to reunion with native NCs just isolated from the in vivo state.

### Example 8

### Cell Isolation from Human Limbus

Corneoscleral rims from human donors (ages 23 to 70) were obtained from the Florida Lions Eye Bank (Miami, Fla.) and managed in accordance with the Declaration of Helsinki. After being rinsed three times with Hank's balanced salt solution, containing 50 mg/ml gentamicin and 1.25 mg/ml amphotericin B, and the removal of excessive sclera, conjunctiva, iris and corneal endothelium, the rim was cut into one-clock-hour segments, each including tissue 1 mm within and beyond the anatomic limbus. Limbal segments were digested with 2 mg/ml collagenase A in serum free ESCM at 37 C for 18 hours under humidified 5% CO₂ to generate collagenase-isolated clusters. In parallel, the limbal segment was digested with 10 mg/ml dispase in ESCM at 4 C for 16 hours to isolate an intact epithelial sheet.

### Serial Passage on Plastic or Coated Matrigel

Single cells derived from collagenase-isolated clusters by 0.25% trypsin and 1 mM EDTA (T/E) at 37 C for 15 minutes were seeded at 1×10⁵ per cm² in the 6-well plastic plate with or without coated Matrigel, which was prepared by adding 40 ul of 5% Matrigel per cm² 1 hour before use and cultured in ESCM containing 4 ng/ml bFGF and 10 ng/ml LIF in humidified 5% CO₂ with medium changed every 3 or 4 days. Cells at 80% or 90% confluence were rendered single cells by T/E and serially expanded at the seeding density of 5×10³ cells per cm² for up to 12 passages. The extent of total expansion was measured by the number of cell-doubling (NCD) using the following formula: NCD=log₁₀(y/x)log₁₀2, where y is the final density of the cells and x is the initial seeding density of the cells. In parallel, cells were cultured in the 6-well plate without coated Matrigel in the same medium as a control.

Co-Culturing with Limbal Epithelial Progenitor Cells in 3D Matrigel

Cells expanded on coated Matrigel at passage 4 (P4) were reseeded in 3D Matrigel to generate P4/3D aggregates. Single cells obtained from P4/3D aggregates or HUVEC were pre-labeled with red fluorescent nanocrystals mixed with singles cells derived from dispase-isolated limbal epithelial sheets at a ratio of 1:4, and seeded at the density of 5×10⁴ per cm² to generate sphere growth. After 10 days of culturing in ESCM, the resultant spheres were collected by digesting Matrigel with 10 mg/ml dispase at 37 C for 2 hours.

Differentiation into Vascular Endothelial Cells

To induce differentiation into vascular endothelial cells, single cells from P4/3D aggregates were seeded at the density of 104 cells per cm2 in 24 well plastic plates for 3 days in the Endothelial Cell Growth Medium 2 (EGM2) supplemented with 10 ng/ml VEGF. At 80-90% confluence, cells were incubated with 10 ug/ml Dil-Ac-LDL for 10 hours at 37 C in the humidified 5% CO₂ incubator and/or fixed with 4% paraformaldehyde for immunofluorescence staining.

### Vascular Tube Formation by HUVEC

Single cells obtained from P4/3D aggregates were mixed at a ratio of 1:1 with red fluorescent nanocrystals, pre-labeled HUVEC and seeded at the density of 10⁵ cells per cm2 on the surface of Matrigel, which was prepared by adding 50 ul of 100% Matrigel into 24 well plates for 30 minutes before use, and cultured in EGM2 to elicit vascular tube-like network. P4/3D cells or HUVEC alone were also seeded at the same density as controls.

### Results

### Serial Passages on Plastic

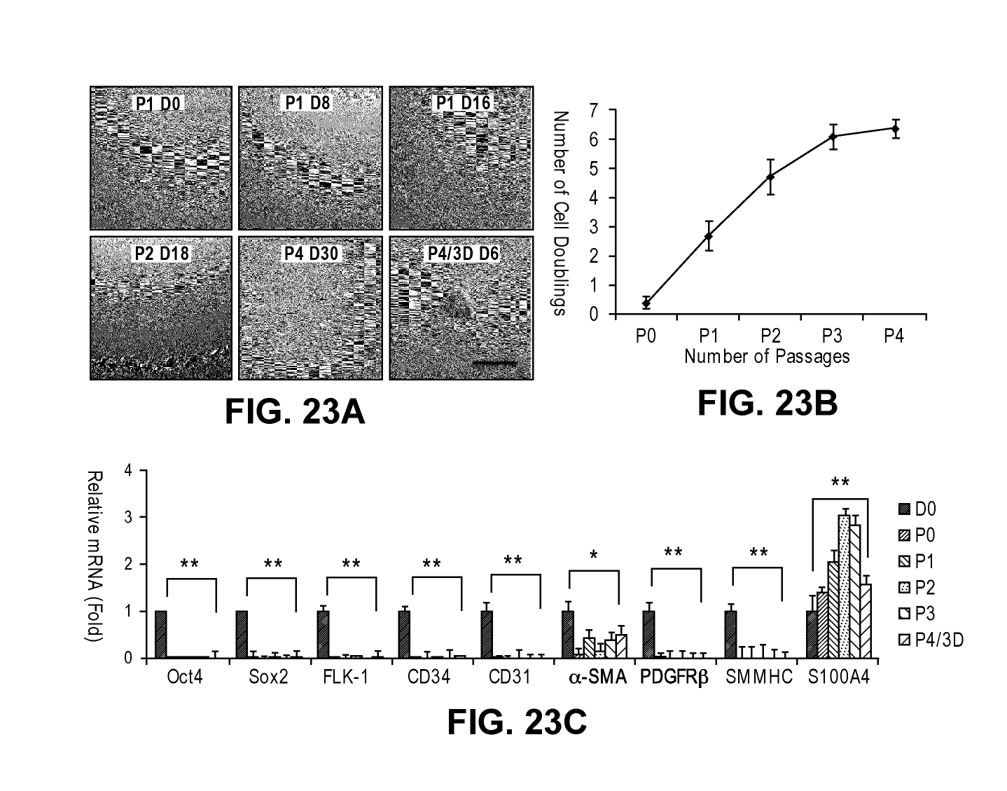

To investigate the significance of Matrigel for such success, we first expanded the limbal NCs on plastic by serial passage in ESCM containing LIF and bFGF using collagenase-isolated cells from a 62 years old donor. Such culture yielded spindle cells (FIG. 23 A) and could only reach P4 with a doubling time of over 165 h and NCD of 6 (FIG. 23 B). When P3 single cells were reseeded in 3D Matrigel for 6 days, they generated P4/3D aggregates at Day 6 with a smooth contour (FIG. 23 A). Compared to D0 cells just isolated, P3 spindle cells did not express Oct4 and Sox2, i.e., markers of ESC. They also did not express Flk-1, CD34, CD31, and PDGFRβ, markers suggestive of angiogenesis progenitors. Because they expressed transcripts of α-SMA and S100A4, but not SMMHC (FIG. 23 C, n=3, *P<0.05, **P<0.01), limbal NCs expanded on plastic turned into myofibroblast. Furthermore, the resultant P4/3D cells did not regain expression of ESC and angiogenesis markers even after being reseeded in 3D Matrigel.

### Serial Passages on Coated Matrigel

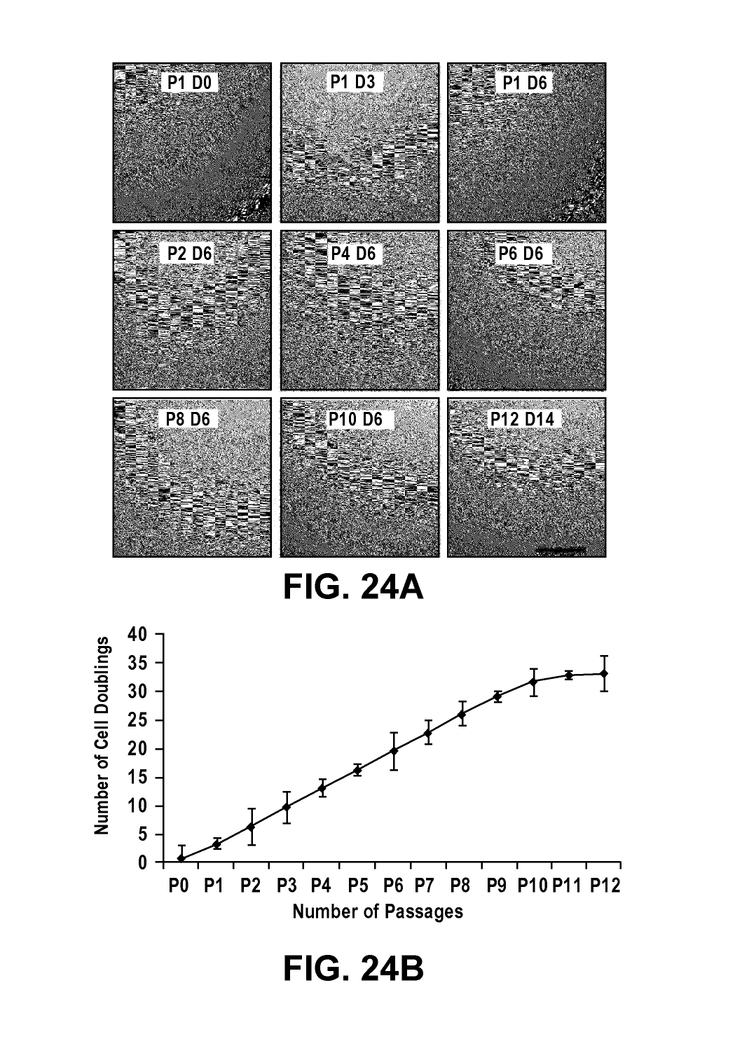

When the above collagenase-isolated cells were serially passaged on coated Matrigel in ESCM with bFGF and LIF spindle shaped cells could be isolated and expanded by completely eliminating epithelial cells by passage 2 (P2) (FIG. 24 A). Unlike the counterpart cultured on plastic, spindle cells could be expanded on coated Matrigel for up to P12, resulting in a total of 33 cell doublings, yielding about 1×1010 spindle cells from 12 limbal segments. Cells at P1 to P10 exhibited a uniform proliferative rate with a cell doubling time between 43 to 47 h (FIG. 24 B, Table 2).

### Expression of Pericyte Markers by Expanded Spindle Cells

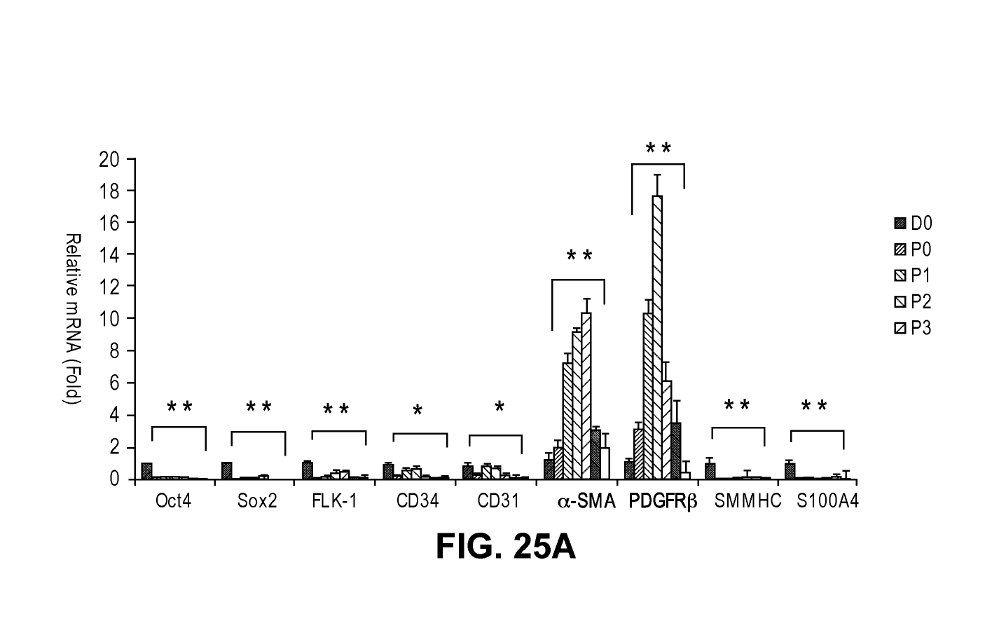

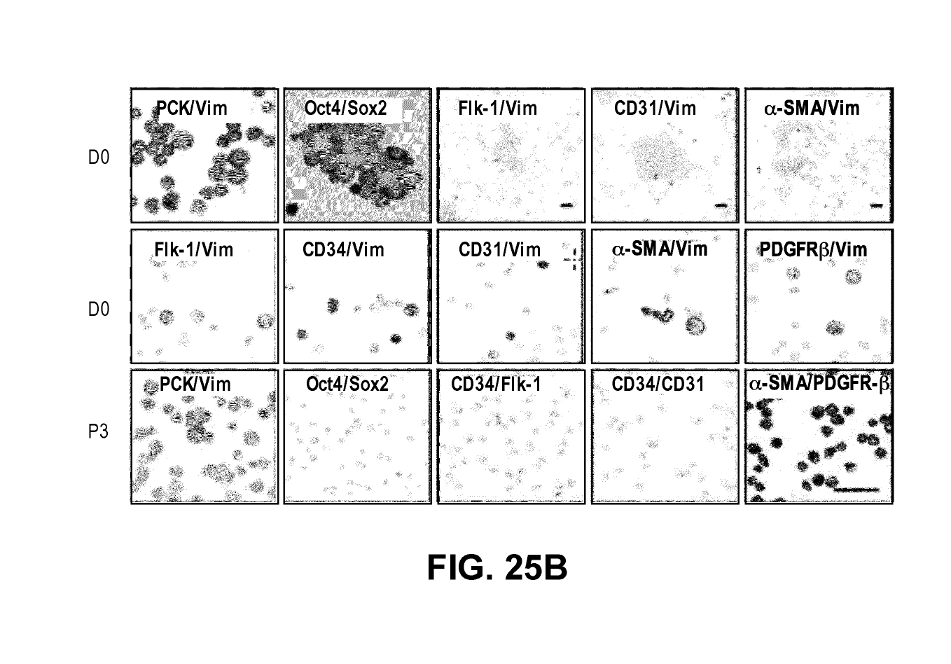

Double immunostaining with antibodies against pancytokeratins (PCK) and vimentin (Vim) showed that collagenase-isolated clusters consisted of approximately 80% PCK+/Vim− epithelial cells and 20% PCK−/Vim+ cells, and that both PCK+ cells and Vim+ cells expressed ESC markers such as Oct4 and Sox2 (FIG. 25 B). Further double immunostaining between the aforementioned markers and Vim showed that Vim+ cells expressed Flk-1, CD34, CD31, and [symbol] -SMA, but the overall percentage of colocalization was less than 1% (n=1000), and none expressed PDGFR® (FIG. 25 B), suggesting that the majority of these Vim+NCs did not express markers suggestive of either endothelial progenitor cells or pericytes. PCK+/Vim-epithelial cells were completely eliminated after P2 as confirmed by the disappearance of p63 and CK12 transcripts and negative staining to PCK and p63. In contrast, spindle cells emerged from P3 and uniformly expressed Vim but not PCK (FIG. 25 B). RT-qPCR showed that expression of Oct4, Sox2, Flk-1, CD34, CD31, SMMHC, and S100A4 transcripts became undetectable, while that of α-SMA and PDGFRβ transcripts were markedly upregulated during serial passage (FIG. 25 A, n=3, P<0.05). Compared to the expression level at D0 when cells were freshly isolated, expression of the α-SMA transcript was markedly elevated till P12 while that of the PDGFRβ transcript was also elevated till P8 (FIG. 25 A). The above pattern of transcript expression was confirmed by immunofluorescence staining. For example, P3 spindle cells did not express Oct4, Sox2, Flk-1, CD34, and CD31, but strongly expressed α-SMA and PDGFRβ (FIG. 25 B). Their lack of expression of SMMHC supported that they were not smooth muscle cells. Their positive expression of α-SMA without S100A4 supported that they were not myofibroblasts. Collectively, these data indicated that expanded spindle cells upregulated their expression of markers suggestive of pericyte differentiation.

### Angiogenesis Progenitors Promoted by Reseeding in 3D Matrigel

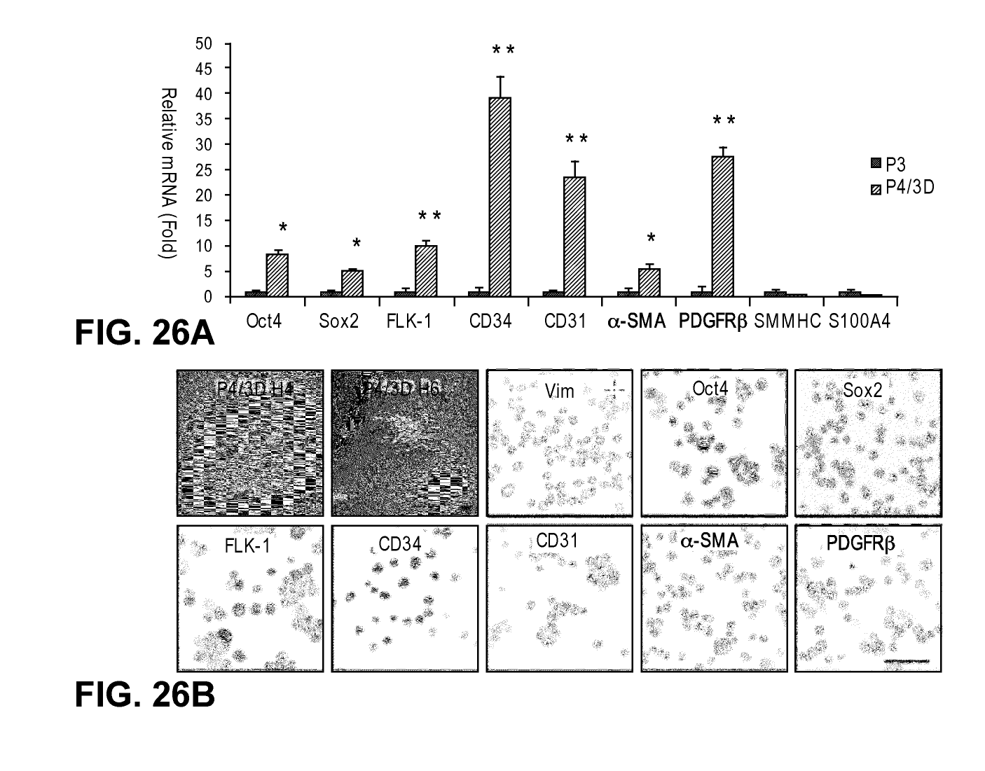

Previously, we discovered that expression of ESC markers could be regained in P3 spindle cells if reseeded in 3D Matrigel. We thus wondered whether expression of markers suggestive of angiogenesis progenitors could be influenced by such a maneuver. Single P3 cells formed cell aggregates as early as 4 h after being re-seeded in 3D Matrigel (FIG. 26 B). At Day 6, these cell aggregates adopted a stellate contour (FIG. 25 B). Consistent with our recent report, expression of Oct4 and Sox2 transcripts by P4/3D D6 aggregates was indeed upregulated to 5 and 8 folds when compared to that expressed by P3 spindle cells expanded on coated Matrigel (FIG. 26 A). Interestingly, expression of Flk-1, CD34, and CD31 transcripts was markedly upregulated by 10 to 40 folds, while that of α-SMA and PDGFRβ transcripts was upregulated by 5 and 27 folds, respectively (FIG. 26 A, n=3, P<0.05). In contrast, expression of SMMHC and S100A4 transcripts remained undetectable in P4/3D D6 cells. Immunofluoroscence staining of single cells released from P4/3D aggregates confirmed positive and uniform expression of Vim and all of the aforementioned angiogenesis markers (FIG. 26 B) but negative expression of SMMHC and S100A4 markers. These results suggested that reseeding back in 3D Matrigel not only restored expression of ESC markers but also promoted expression of markers suggestive of angiogenesis progenitors in the direction of pericytes but not smooth muscle cells or myofibroblasts.

Differentiation into Vascular Endothelial Cells

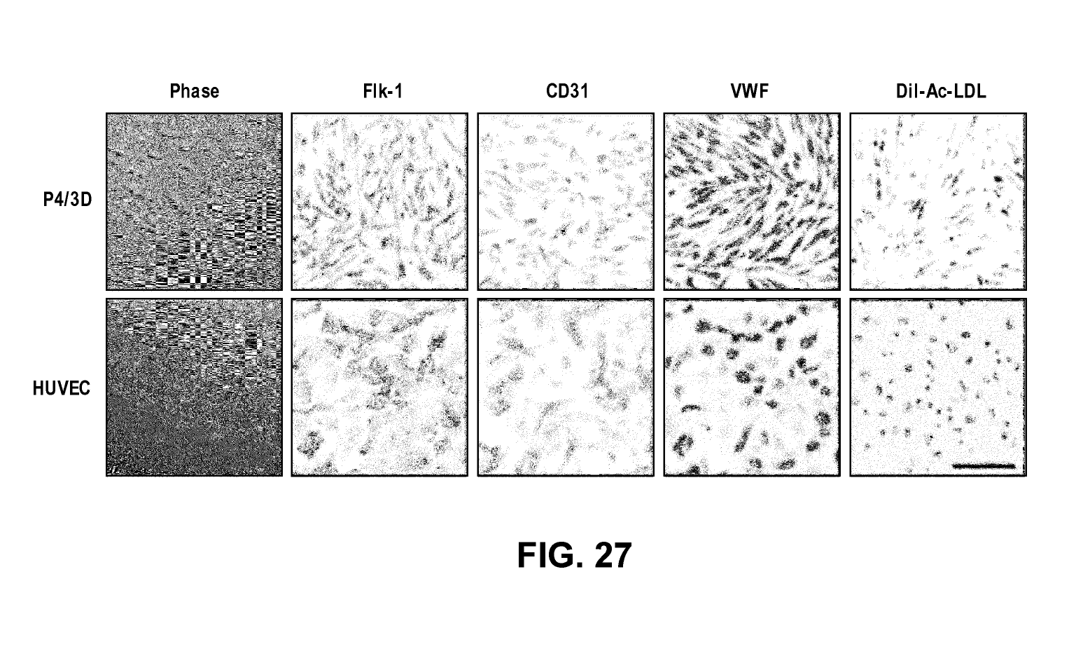

To confirm that the aforementioned P4/3D cells were indeed angiogenesis progenitors, cells were released from 3D Matrigel by dispase digestion, rendered into single cells by T/E, and seeded on plastic in EGM2 supplemented with 10 ng/ml VEGF-A. After three days of culturing, the resultant cells exhibited spindle cells similar to HUVEC (FIG. 27). They also expressed positive immunofluorescence staining to Flk-1, CD31, and vWF and took up Dil-Ac-LDL (FIG. 27, top) in a similar fashion to the positive control of HUVEC (FIG. 27, bottom). These data indicated that P4/3D cells indeed could differentiate into vascular endothelial cells.

### Support of HUVEC Formed Vascular Tube Network

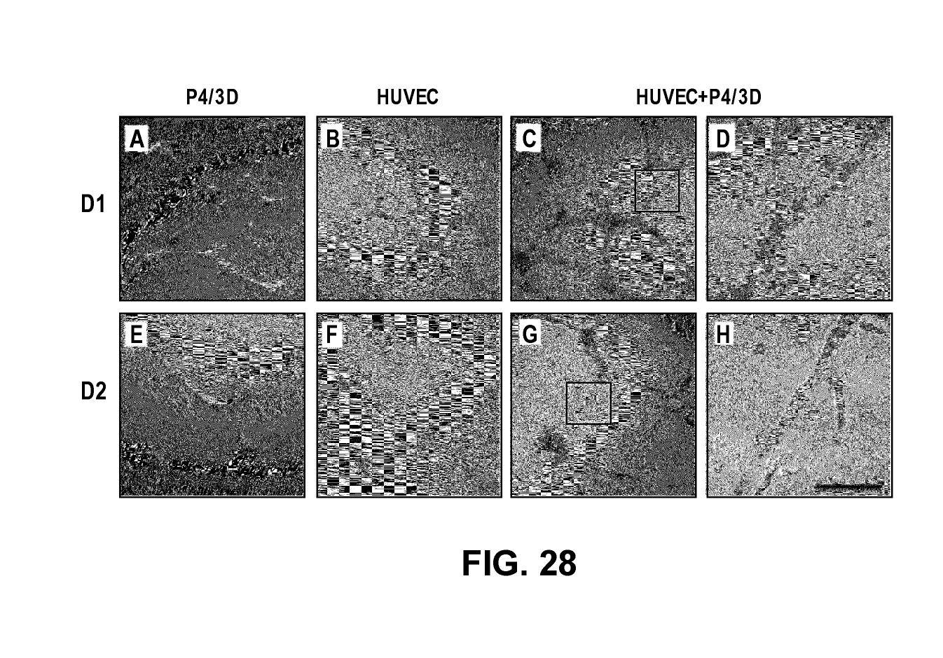

One important step in the process of angiogenesis is to stabilize the vascular network formed by vascular endothelial cells by pericytes. To confirm that P4/3D cells were indeed angiogenesis progenitors, we examined whether they also possessed the phenotype of pericytes. To recapitulate such a function of pericytes, we seeded single HUVEC, single P4/3D cells, and a combination of both on the surface of 100% Matrigel in EGM2 as previously reported. Both single P4/3D cells and pre-labeled (red) HUVEC formed networks at Day 1 (FIG. 28, A and B). However, such networks were largely disintegrated by Day 2 (FIGS. 28, E and 5F). In contrast, the network formed by cocultured P4/3D cells and HUVEC (FIG. 28 C) was maintained at Day 2 (FIG. 28 G) and Day 5. Higher magnification of such network confirmed tight adherence of P4/3D cells onto HUVEC (red) (FIG. 28, D and H). These results confirmed that P4/3D cells indeed possessed the pericyte phenotype to stabilize the vascular tube like network formed by HUVEC.

### Prevention of Differentiation of Limbal Epithelial Progenitors

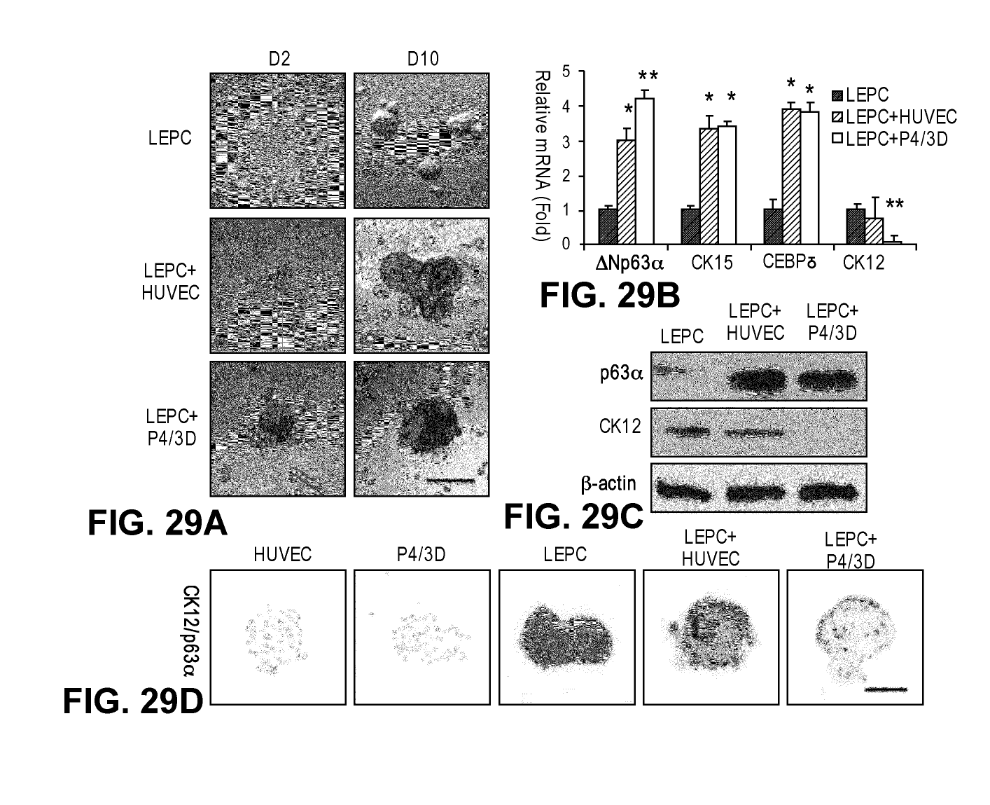

Compared to PCK+ cells in collagenase-isolated clusters, those in dispase isolated sheets express less p63α and CK15, but more CK12. Thus, dispase isolated more differentiated limbal epithelial progenitor cells (LEPC) than collagenase based on the findings that p63 (signifies limbal basal progenitors including SC, CK15 is expressed by limbal basal epithelial cells, and CK12 is a marker of corneal epithelial differentiation. Single PCK+ epithelial cells and Vim+ stromal cells from collagenase-isolated clusters could reunite to generate sphere growth in 3D Matrigel and such reunion helps to maintain epithelial clonal growth and prevent corneal epithelial differentiation. We thus examined whether LEPC obtained from dispase-isolated epithelial sheets could also form reunion with pre-labeled (red) P4/3D cells or HUVEC in 3D Matrigel. As shown in FIG. 28, reunion indeed occurred at Day 2 and gradually developed into a larger sphere by Day 10 similar to those formed by LEPC alone (FIG. 29A). Compared to spheres formed by LEPC alone, spheres formed by LEPC+HUVEC and LEPC+P4/3D had significantly higher transcript expression of ΔNp63α, CK15, and CEBPδ, of which the latter plays a role in maintaining quiescence of limbal epithelial SCs (FIG. 29 B, n=3, all P<0.05). Expression of the CK12 transcript by LEPC+HUVEC was not different from LEPC alone (FIG. 29 B, n=3, P>0.05), but that by LEPC+P4/3D was significantly reduced to an undetectable level (FIG. 29 B, n=3, P<0.01). Western blot analysis confirmed that the protein level of p63 (was elevated to 6.5 and 6.1 folds in LEPC+HUVEC and LEPC+P4/3D respectively, when compared to LEPC alone (FIG. 29 C, n=3, P<0.05). The protein level of CK12 was not changed in LEPC+HUVEC (FIG. 29 C, n=3, P>0.05) but was reduced to an undetectable level in LEPC+P4/3D (FIG. 29 C, n=3, P<0.01). Double staining with p63α and CK12 also confirmed that HUVEC or P4/3D cells alone did not express p63α or CK12, and that CK12 was expressed by LEPC alone and LEPC+HUVEC, but not LEPC+P4/3D (FIG. 29 D). Collectively, these findings indicated that although both the P4/3D cells and HUVEC could join with LEPC to generate sphere growth in 3D Matrigel to promote expression of epithelial progenitor/SC markers, the former but not the latter could prevent differentiation of LEPC.

### Example 9

Adipose tissue is processed and isolated as follows: (1) Wash adipose tissue 3 times with BSS, (2) Cut tissue into fine pieces ˜2×2 mm, and subdivide them into two parts, (3) Subject one part to digestion with 1 mg/ml of collagenase A in DMEM/10% FBS and the other in MESCM for 16 h at 37 C, (4) Centrifuge the digest at 300×g for 10 min to collect the pellet that contains SVF cells, and collect both floating cells (FC), (5) Resuspend pellet cells in either DMEM/10% FBS (the first part) or MESCM (the second part), respectively, (6) Filter the cell suspension via a 250 μm mesh filter for both parts, and collect cells flowing through and those not as two fractions, (7) Add the RBC lysis buffer to the fraction with flow through and centrifuge at 300×g for 10 min to collect cells for the flow through fraction. For the above cell fractions, RNAs will be collected from cell extracts, and used for qRT-PCR analysis of the following transcript expression: ESC markers (Oct4, Nanog, Rex1, Sox2, nestin, ALP, and SSEA4) and other marker such as CD34, CD31, VWF, aSMA, PDGFRβ, CD146, and NG-2.

| | Digestion Collagenase | |
|---|---|---|
| Exp Group | Medium | Cell Fraction |
| 1 | DMEM/10% FBS | SVF (flow through and not) |
| 2 | DMEM/10% FBS | FC (floating cells) |
| 3 | MESCM | SVF (flow through and not) |
| 4 | MESCM | FC (floating cells) |

### Example 10

### Angiogenesis Progenitors can be Better Maintained and Expanded on Coated Matrigel in SHEM than Plastic in SHEM

How to expand hAMSC from the compact stroma of the AM that contains more cells expressing angiogenesis markers was investigated. Specifically single cells derived from the C/D Method are cultured on coated MATRIGEL® or plastic (PL) in SHEM at the seeding density of 5×10⁴/cm2. Cells are subpassaged every 8 days until they lost the proliferative potential. During passaging, mRNA and cytospin were collected for analysis.

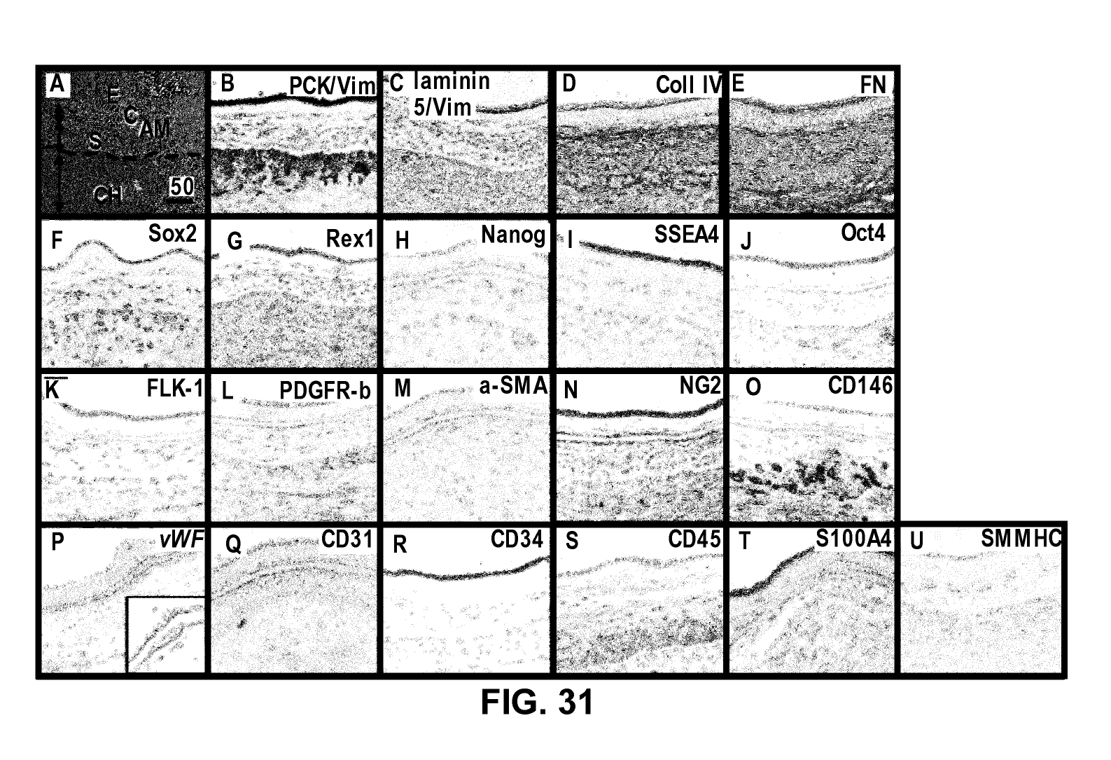

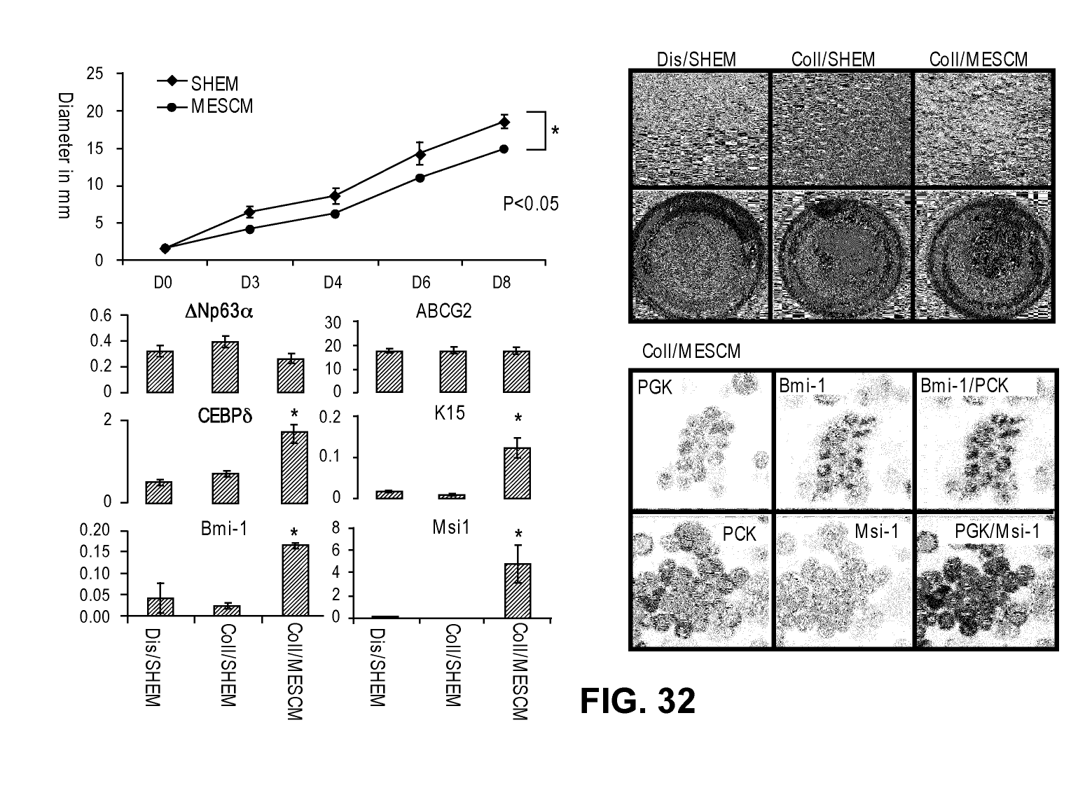

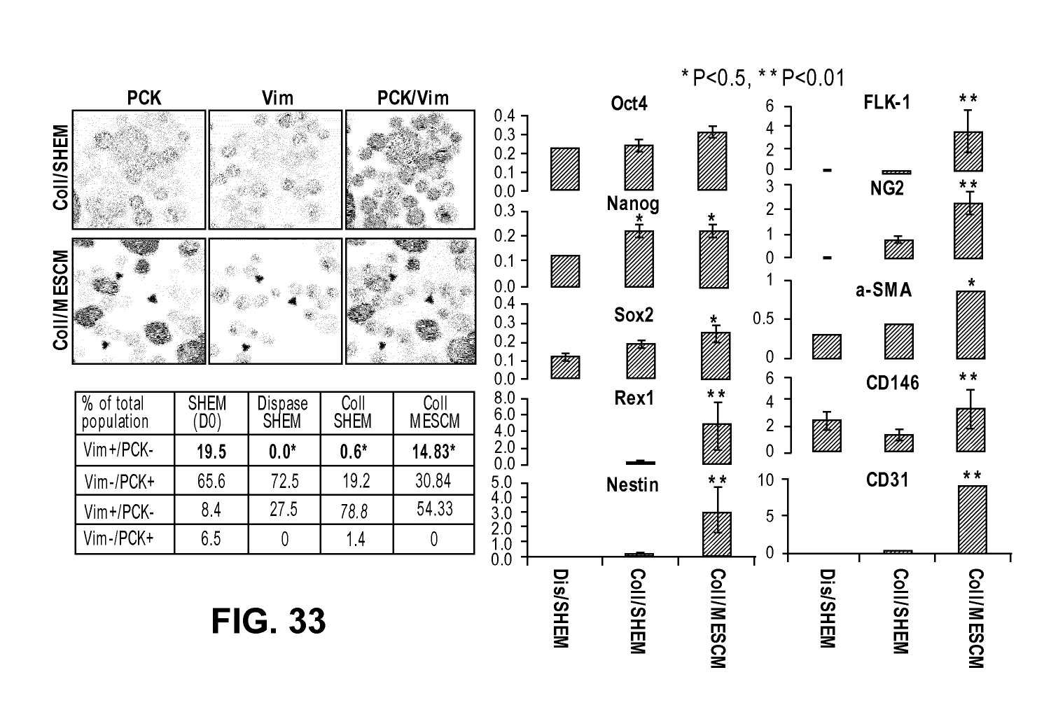

FIG. 31 shows that cells expanded on coated MATRIGEL® are smaller cells in size, have a greater cumulative doubling times, and can be expanded up to 5 passages, resulting in a total of 2.4×10⁶ cells, while cells expanded on plastic in D/F on PL can only be expanded up to 3 passages. At P3, cells culture in D/F on PL were enlarged and cease proliferation. Furthermore, cells culture on coated MATRIGEL® in SHEM express stronger expression of angiogenesis markers such as FLK-1+, PDGFR-β, vWF, α-SMA and some CD146 than PL.

### Example 11

### Limbal Stromal Cells Isolated by Collagenase Digestion are Small and Heterogeneously Express ESC Markers and Angiogenesis Markers, and Expression of Such Markers Decreases if Digestion is Performed in SHEM but not in MESCM, which Contains bFGF and LIF

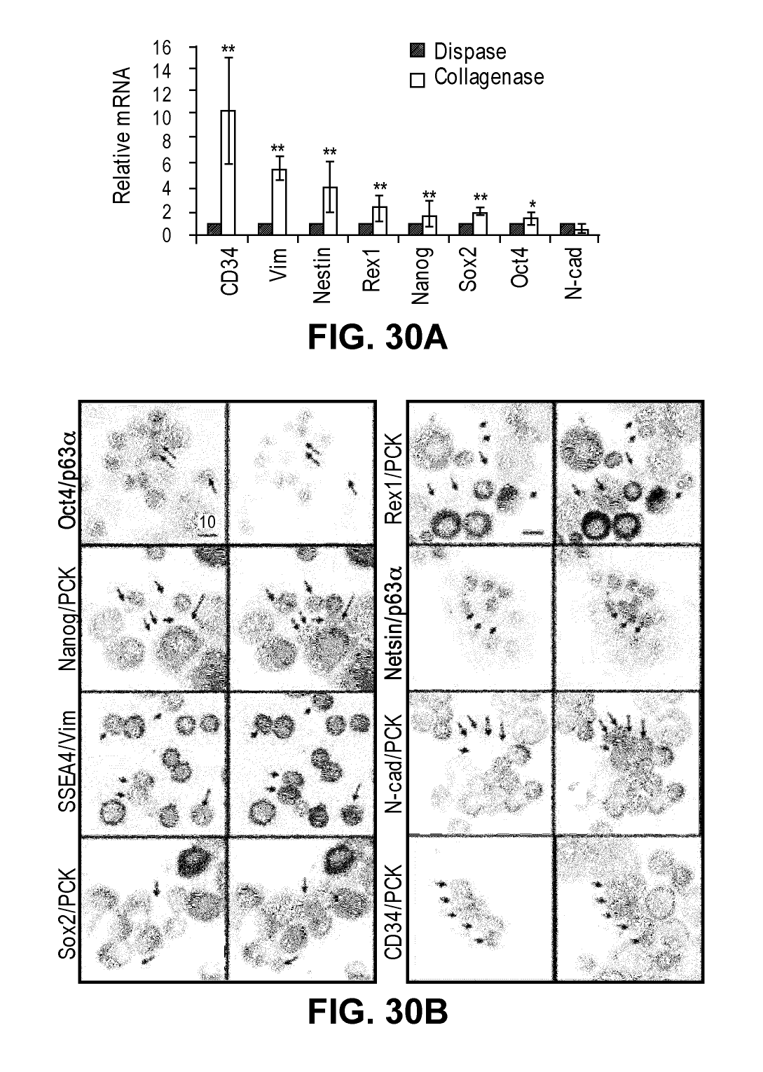

Cornea limbal epithelial progenitor cells lie deep in limbal basal of crypt-like structures in limbal palisades of Vogt. FIG. 15 shows that digestion of collagenase preserves the basement membrane components, such as laminin 5 in a cluster. Importantly, collagenase isolated clusters generate more small pancytokeratin−/p63α−/vimentin+ cells with the size as small as 5 μm in diameter and heterogeneously expressing some embryonic markers Oct4, Sox2, Nanog, Rex1, Nestin, N-cadherin, SSEA4 and CD34 (FIG. 30). Digestion with D/C method also yields cells expressing angiogenesis markers such as CD31, FLK-1, PDGFRβ and α-SMA (FIG. 8). When The present inventors investigate the effects of digestion medium, if containing FBS, which commonly use to expand MSC versus our MESC medium, on the function role (determine by phenotype expressions) of ESC markers. During digestion period, collagenase isolated cluster digest in MESCM significantly express more ESC markers than SHEM or conventional medium used to isolated MSC, i.e., DMEM+10% FBS (DF). qRT-PCR shows that transcript expression of both ESC markers and angiogenesis markers are markedly declined in SHEM or DF (FIG. 30).

### Example 12

### Successful Expansion of Limbal Stromal Cells on Coated and 2D but not 3D MATRIGEL® in MESCM Containing bFGF and LIF Using Either Collagenase- or D/C-Isolated Cells. The Resultant Expanded Stromal SCs Express Markers of Both Angiogenesis Progenitors and MSC. In Contrast, D/C-Isolated Cells Lose Such a Phenotype if Expanded on Plastic in MESCM

FIG. 17 shows how limbal stromal cells can be successfully expanded up to passage 4 on coated MATRIGEL®. Thickness of MATRIGEL® defined by coated and thick (3D), are prepared by adding to plastic dish with 5% diluted MATRIGEL®, and 200 μl of 50% diluted MATRIGEL® (all in DMEM) per cm2, respectively by incubation at 37 C for 1 h before use. Limbal stromal stem cells are cultured on coated MATRIGEL® in modified ESCM expansion medium (MECM) consisting of DMEM/F-12 (1:1) supplemented with 10% knockout serum (Invitrogen, USA), b-FGF (4 ng/ml), insulin (5 μg/ml), transferring (5 μg/ml), sodium selenite (5 ng/ml) (Sigma, USA) and human LIF (10 ng/ml) (Chemicon, USA) for 6 days before further passage. The proliferative activity measured by nuclear EdU labeling on Day 5 for 24 h before termination. Only the spindle cells emerged from coated MATRIGEL®, rapidly increased in number upon for further passages. In contrast, 3D MATRIGEL® generates spheres. The proliferative labeling index confirmed the positive proliferation of 25.6±3.2% in PCK+ cells and 13.6±1.5% in PCK− cells in coated MATRIGEL®, are significantly higher than 12.5±2.0% in PCK+ cells and 2.6±1.2% in PCK− cells in 3D MATRIGEL®. This method successfully eliminate epithelial contamination as evidenced by rapid disappearance of epithelial markers by Passage 2.

FIGS. 24 and 25 shows that successful expansion of limbal stromal SCs can also be achieved by culturing D/C-isolated cells on coated MATRIGEL® in MESCM. Similar to collagenase-isolated cells, this method achieves successful expansion of up to 12 passages and more than 33 doubling times yielding about 1×1010 spindle stromal cells. The expanded limbal stromal SCs express less ESC markers such as SSEA-4, OCT-4, Nanog, and Rex1, but increases expression of angiogenesis (pericyte) markers such as FLK-1, CD31, PDGFRβ, α-SMA and CD34 and MSC markers such as CD73, CD90, and CD105.

To evaluate significance use of coated MATRIGEL® in the aforementioned successful expansion of limbal stromal SCs expression both angiogenesis progenitor and MSC markers, we compare to D/C-isolated cells cultured on plastic substrate containing MESCM. FIG. 6 shows that D/C cells can only expanded to 4 passages with a total number of cell doubling time of 6. qRT-PCR shows the expressions of ESC markers such as Oct4, Nanog, Sox2 and angiogenesis markers such as FLK-1, CD31 and CD34 is significantly decline. There is an increasing expression of α-SMA and S100A4, but significant reduction of PDGFRβ and SMMHC, suggesting that cells expanded on plastic turn into myofibroblasts even if they are cultured in MESCM.

### Example 13

### D/C Isolated Limbal Cells Expanded on Coated MATRIGEL® in MESCM have the Hither CFU-F and Potential of Differentiating into Tri-Lineage of Osterocytes, Chondrocytes and Adipocytes than the Same Cells Expanded on Plastic in DMEM+10% FBS, a Conventional Method of Expanding MSC

To compare the potency of serving as MSC, we compared CFU-F, an in vitro way of evaluating MSC function, and differentiation into the known tri-lineage of osteogenic, chondrogenic, and adipogenic lineages. FIG. 12 shows that D/C cells expanded on coated Matrigel in MESCM at P4 (D/C) exhibited the highest CFU-F, judged by either a total or by three different clones when compared to D/C DF or RSC DF cells expanded at P4 (FIG. 12a, P<0.05, n=3). The CFU-F of D/C DF cells is significantly higher than that of RSC DF cells (P<0.05, n=3). When these three cells are cultured in the medium designated for adipogenesis, osteogenesis, and chondrogenesis, respectively, the result shows that although they all could differentiate into adipocytes, osteocytes, and chondrocytes, D/C cells expanded on coated MATRIGEL are significantly more potent than D/C and RSC cells expanded on plastic in DF (FIGS. 13B and 13C). There was no difference in adipogenesis and osteogenesis between D/C DF and RSC DF cells, but D/C DF cells were more potent than RSC DF cells in chondrogenesis.

### Example 14

### Regain of Expression of ESC by Expanded Limbal Stromal SCs the Express Angiogenesis and MSC Markers by Re-Seeding in 3D MATRIGEL® in MESCM and they Serve as Angiogenesis Progenitors by Differentiating into Vascular Endothelial Cells and Serving as Pericytes. In Contrast, Cells expanded on Plastic in MESCM Remain Myofibroblasts

Although the aforementioned limbal stromal SCs expanded on coated MATRIGEL® in MESCM lose expression of ESC markers, they regain the expression of ESC markers and increase the expression of angiogenesis markers by being re-seeded in 3D MATRIGEL®. FIG. 8A shows that after the above cells at P4 are reseeded in 3D MATRIGEL® in MESCM, they turn into cell aggregates (spheres). The expression of ES markers Oct4, Nanog, Sox2, Rex1, and angiogenic markers FLK-1, CD31, and CD34, are significantly reversed to native phenotype when compared to P3 cells on coated MATRIGEL® (P<0.01, n=3, compared to P3 cells, and P>0.05, compared to P0). In contrast, cells expanded on plastic in MESCM or on coated MATRIGEL® but in SHEM or DF still do not re-express ESC or angiogenesis progenitor markers. They continues to express α-SMA and S100A4, suggesting that they are irreversibly differentiate into myofibroblasts.

FIGS. 27 and 28 shows that expanded limbal stromal SCs serve as angiogenesis progenitors because they can differentiate into vascular endothelial cells and pericytes by supporting the vascular network formed by human umbilical vein endothelial cells (HUVEC). Specifically, they are cultured in the Endothelial Cell Growth Medium 2 (EGM2) supplemented with 10 ng/ml VEGF. At 80-90% confluence, cells are incubated with 10 μg/ml Dil-Ac-LDL for 10 h at 37° C. and fixed with 4% paraformaldehyde for immunofluorescence staining. The result shows that expanded limbal stromal SCs exhibit a phenotype similar to the control, i.e., HUVEC, exhibiting positive immunoflourscence staining of FLK-1, CD31, vWF and took up Dil-Ac-LDL. To test whether expanded limbal stromal SCs have the pericyte function to support vascular tube network, the expanded limbal MSC obtained from P4/3D aggregates were mixed at a ratio of 1:1 with red fluorescent nanocrystals (Qtracker® cell labeling kits) pre-labeled HUVEC and seeded at the density of 10⁵ cells per cm² on the surface of 3D MATRIGEL®. Vascular tubes like formation are monitored on 12 h, Day 1, Day 2 and Day 5. The network formed by HUVEC or limbal MSC disintegrated by Day 2. In contrast, the network formed by co-culture can be further maintained at Day 2 and Day 5.

### Example 15

### Limbal Stromal SCS Expanded on Coated MATRIGEL® and Switched to 3D MATRIGEL® in MESCM can Serve as Niche Cells to Support the Sternness of Limbal Epithelial Progenitor Cells, while Cells Expanded on Plastic in MESCM or DMEM+10% FBS Cannot

Specifically, HUVEC or limbal MSC are added to single cells derived from dispase isolated epithelial sheets to form spheres on 3D MATRIGEL®. By Day 10, spheres are harvested for protein and mRNA analysis. FIG. 29 shows that both protein and mRNA expression by LEPC+HUVEC and LEPC+limbal MSC had significantly higher transcript expression of epithelial pronitors markers ΔNp63, CK15, and CEBPδ (n=3, all P<0.05). Expression of the CK12 transcript by LEPC+HUVEC was not different from LEPC alone, but was significantly reduced to an undetectable level in LEPC+limbal MSC. Because the phenotype of expressing ES markers can be regained by reseeding in 3D MATRIGEL® for limbal stromal SCs that have been expanded on coated MATRIGEL® in MESCM but not in SHEM or DF or expanded on plastic in MESCM. On day 10, sphere formed by dispase-isolated epithelial cells with addition of limbal stromal SCs result in spheres expressing 3.9 fold more p63α and 0.5 fold less cornea differentiation CK12. In contrast, addition of cells expanded in DF result in sphere expressing 0.7 fold p63α and 0.7 fold of CK12. The colony forming efficiency on growth-arrested 3T3 feeder layers is significantly higher by limbal stromal SCs expanded on coated MATRIGEL® in MESCM than cells expanded on coated MATRIGEL® but in DF.

### Example 16

### Expression of ESC and Angiogenesis Markers by hAMSC and hAMEC from AM In Vivo

Because amniotic membrane is avascular tissue, we sought to illustrate the presence of vascular progenitors located in upper region of AM in vivo. Specifically, a 1×1 cm2 of intact amnion/chorion tissue is embedded and sectioned with 6 μm thickness. Immunofluorscence tissue are subjected antibodies against basement membrane (laminin 5, CollIV, Lumican, Keratan sulfate), embryonic markers (Nanog, Sox2, Rex1 and SSEA4) and angiogenic markers (NG2, PDGFR-B, α-SMA, CD133/2, FLK-1, vWF, CD34, CD31 and CD146) and MSC markers (CD90, CD73, and CD105). FIG. 10 shows that AM consists of a single layer of hAMEC and basement membrane lie between stromal layers. Basement components, such as laminin 5 solely express below epithelial cells. Double staining of pancytokeratin (PCK) and vimentin (Vim) confirms their coexpression in hAMEC. Such PCK+ cells heterogeneously express such ESC markers as Oct4, Sox2, SSEA4, Rex1 with weak expression of nanog in some strong PCK+ cells. hAMEC against angiogenic markers showed positive staining to FLK-1, NG2, vWF, CD31 and CD34 and PDGFR-β but negative to α-SMA. hAMEC express strong s100A4 but not SMMHC. The hAMSCs uniformly express Sox2 and Rex1 while Oct4, Nanog, nestin, weakly express in compact layers and cells in the spongy layer do not express Nanog, SSEA4 and Oct4. hAMSCs uniformly express NG2, while FLK-1, vWF, CD31, PDGFR-B and α-SMA are preferentially expressed in the compact but not spongy layer of stromal region. Similarly, MSC markers are also preferentially express in the compact but not spongy layer of stromal region. These results confirm the expression of ESC, angiogenesis progenitors, and MSC markers by both hAMSC and hAMEC in vivo.

### Example 17

### Enrichment and Isolation of AM Stromal SCs by the Novel C/D Method, which Promotes Expression of ESC and Angiogenesis Markers of Isolated hAMSC

Previously, others have isolated hAMSC by different enzymatic digestion methods (summarized in Table 4). The presence inventor seeks to develop a novel isolation method that can separate the upper region of AM stroma from lower region of sponge layer by collagenase follow by dispase method, termed C/D Method.

Specifically, hAM is precut to the size of 4×4 cm². Some pieces are subjected to the conventional method by digestion with 0.25% trypsin/EDTA (T/E) at 37° C. for 5 min followed by digestion with 10 mg/ml of dispase 30-60 min at 37° C. on a shaker. The remaining stromal tissue is subjected to 2 mg/ml collagenase with HAase (200 ug/ml) in digestion medium at 37° C. for 16 h. This conventional method is termed D/C Method and has been used by others (Table 4).

We have developed a new method, termed C/D method, but first submitting some pieces to digestion with 1 mg/ml collagenase with HAase (200 ug/ml) for 16 h at 37° C. The floating sheets that contain AM epithelial sheet and the underlying hAMSC are transferred to a plate containing 10 mg/ml of dispase at 37° C. for 30-60 minutes. All retrieved epithelial sheet from both isolation methods are subjected to TrypLE for 15 mins. The retrieved hAMSC are collected to compare mRNA level for expression of angiogenic markers. Flat mount preparation prior dispase digestion are fixed with 4% paraformaldehyde for immunofluorescence staining

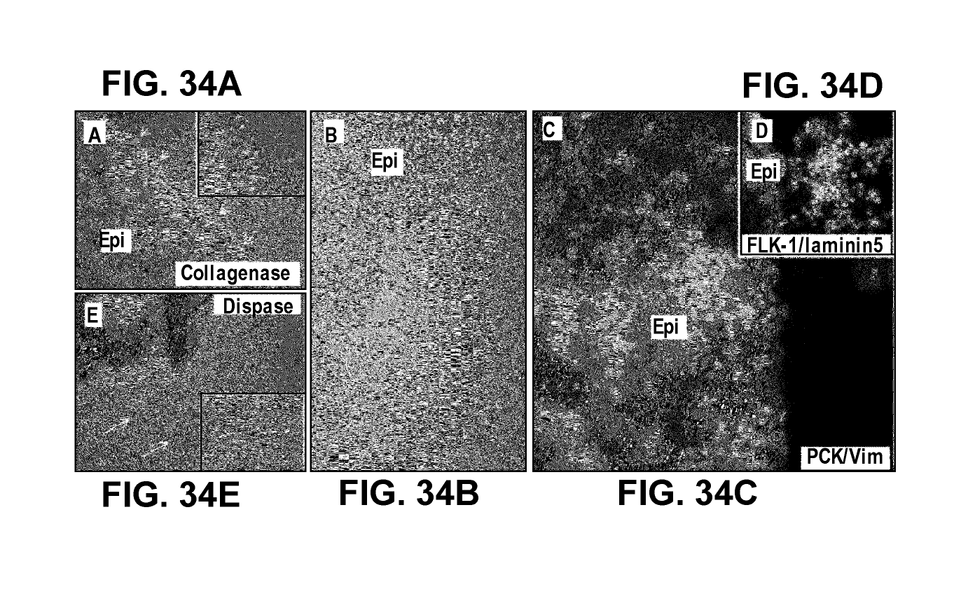

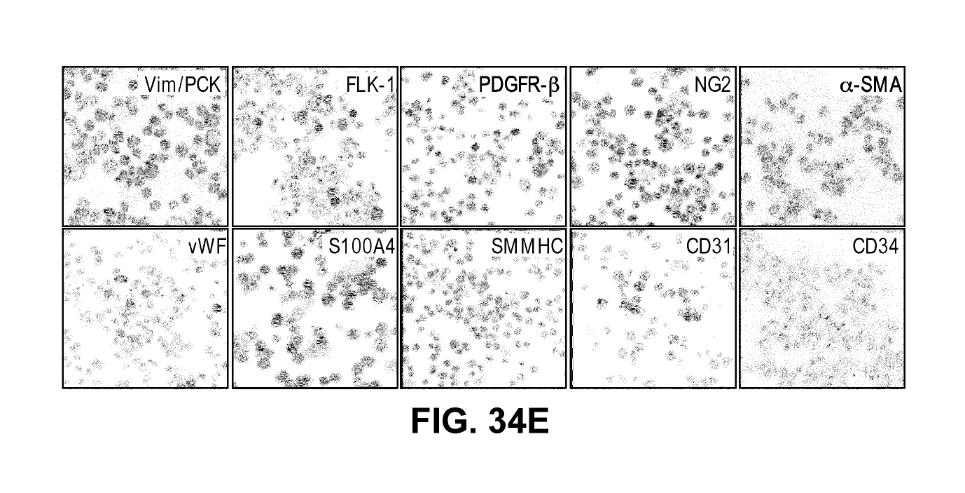

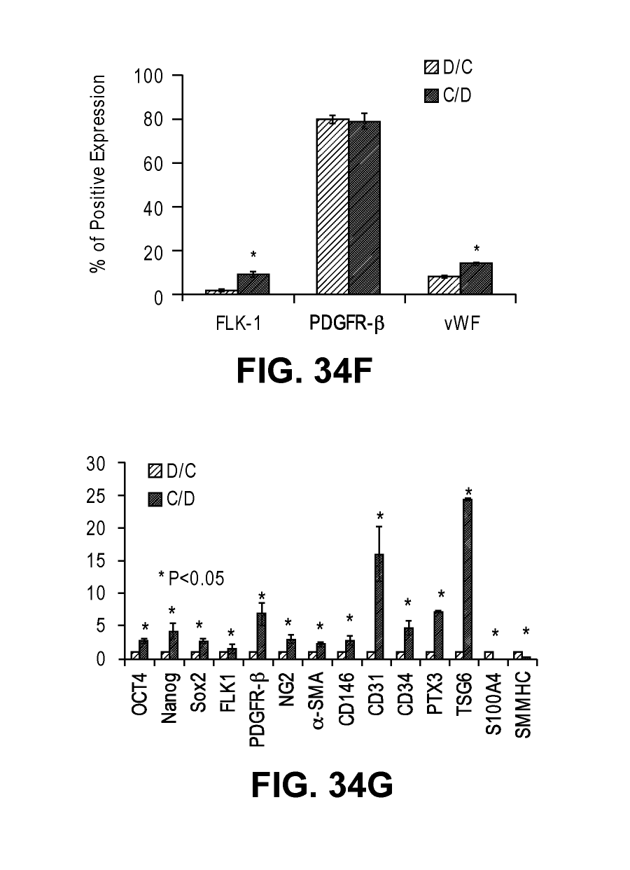

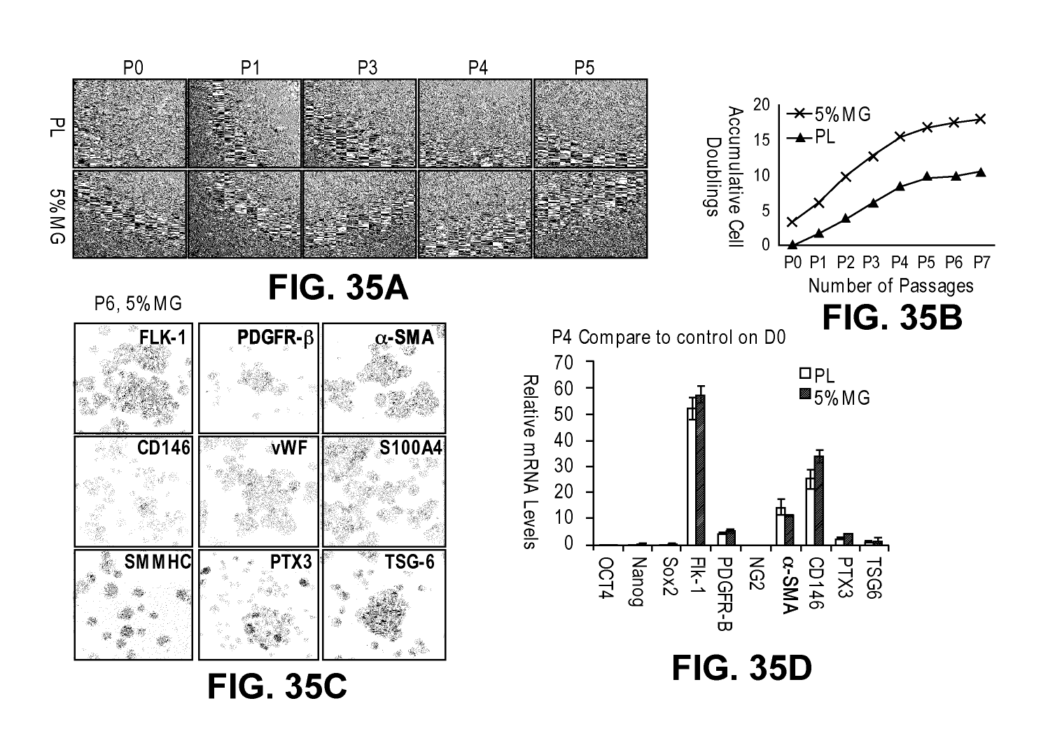

FIG. 34 shows that double immunostaining of both PCK and Vim confirms that <1% of PCK are presented in both C/D and D/C methods. When compare to D/C derived hAMSC, mRNA expressions of ESC markers (Oct4, Nanog, and Sox2) and angiogenesis markers (FLK1, PDGFR-β, NG2, α-SMA, CD146, and CD31) are significantly higher in the C/D Method than the D/C Method. Furthermore, the C/D Method yields a higher percentage of cells expressing FLK-1 and vWF than the D/C Method. Immunostaining further confirms that Vim+ cells also express angiogenesis markers such as NG2, PDGFR-13, FLK-1, vWF and α-SMA, and that isolated cells express low CD34 but strong S100A4 but not SMMHC.

### Example 18

### hAMSC Expanded on Coated MATRIGEL® in SHEM Express More Angiogenesis Progenitor Makers than Those Cultured on Plastic in DMEM+10% FBS, i.e., the Conventional Method for Expanding MSC

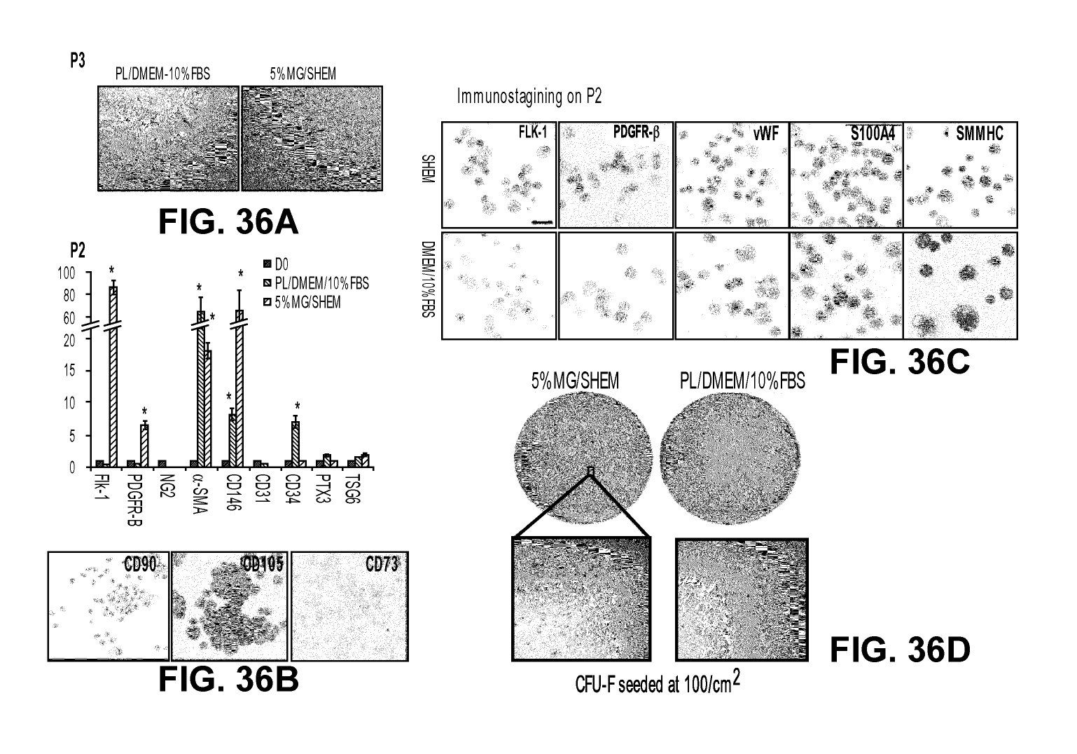

FIGS. 36 and 36 shows that cells cultured on plastic in DMEM/10% FBS (DF), i.e., the conventional method of expanding MSC from hAMSC (Table 2) show significantly lower expression of all angiogenesis progenitor markers, except CD34, α-SMA, and CD146 than cells expanded on coated MATRIGEL® in SHEM, where the expression of FLK-1, PDGFR-13, α-SMA and CD146 is significantly upregulated. Immunostaining further confirms the low expression of FLK-1 and PDGFR-β in cells cultured on plastic in DMEM/10% FBS.

### Example 19

### Use of MSC as Niche Cells

Amniotic membrane is digested with 2 mg/ml collagenase A in MESCM at 37 C for 18 hours under humidified 5% CO₂ to generate collagenase-isolated clusters.

Single cells derived from collagenase-isolated clusters by 0.25% trypsin and 1 mM EDTA (T/E) at 37 C for 15 minutes are seeded at 1×10⁵ per cm² in the 6-well plastic plate with or without coated Matrigel, which was prepared by adding 40 ul of 5% Matrigel per cm² 1 hour before use and cultured in ESCM containing 4 ng/ml bFGF and 10 ng/ml LIF in humidified 5% CO₂ with medium changed every 3 or 4 days.

Cells at 80% or 90% confluence are rendered single cells by T/E and serially expanded at the seeding density of 5×10³ cells per cm² for up to 12 passages.

MSCs expanded on coated Matrigel at passage 4 (P4) are reseeded in 3D Matrigel to generate P4/3D aggregates. Single cells obtained from P4/3D aggregates are mixed with epithelial stem cells. The MSCs act as niche cells for the epithelial progenitor cells which grow into a suitable tissue graft.

### Example 20

### Use of MSC as Niche Cells

Amniotic membrane is digested with 2 mg/ml collagenase A in MESCM at 37 C for 18 hours under humidified 5% CO₂ to generate collagenase-isolated clusters.

Single cells derived from collagenase-isolated clusters by 0.25% trypsin and 1 mM EDTA (T/E) at 37 C for 15 minutes are seeded at 1×10⁵ per cm² in the 6-well plastic plate with or without coated Matrigel, which was prepared by adding 40 ul of 5% Matrigel per cm² 1 hour before use and cultured in ESCM containing 4 ng/ml bFGF and 10 ng/ml LIF in humidified 5% CO₂ with medium changed every 3 or 4 days.

Cells at 80% or 90% confluence are rendered single cells by T/E and serially expanded at the seeding density of 5×10³ cells per cm² for up to 12 passages.

MSCs expanded on coated Matrigel at passage 4 (P4) are reseeded in 3D Matrigel to generate P4/3D aggregates. Single cells obtained from P4/3D aggregates are mixed with epithelial stem cells. The MSCs act as niche cells for the epithelial progenitor cells which grow into a suitable bone graft.

### Example 21

### Use of MSC as Niche Cells

Amniotic membrane is digested with 2 mg/ml collagenase A in MESCM at 37 C for 18 hours under humidified 5% CO₂ to generate collagenase-isolated clusters.

Single cells derived from collagenase-isolated clusters by 0.25% trypsin and 1 mM EDTA (T/E) at 37 C for 15 minutes are seeded at 1×10⁵ per cm² in the 6-well plastic plate with or without coated Matrigel, which was prepared by adding 40 ul of 5% Matrigel per cm² 1 hour before use and cultured in ESCM containing 4 ng/ml bFGF and 10 ng/ml LIF in humidified 5% CO₂ with medium changed every 3 or 4 days.

Cells at 80% or 90% confluence are rendered single cells by T/E and serially expanded at the seeding density of 5×10³ cells per cm² for up to 12 passages.

MSCs expanded on coated Matrigel at passage 4 (P4) are reseeded in 3D Matrigel to generate P4/3D aggregates. Single cells obtained from P4/3D aggregates are mixed with epithelial stem cells. The MSCs act as niche cells for the epithelial progenitor cells which are transplanted into an individual with an epithelial stem cell deficiency.

### Example 22

### Use of MSC as for the Treatment of Chronic Wounds

Amniotic membrane is digested with 2 mg/ml collagenase A in MESCM at 37 C for 18 hours under humidified 5% CO₂ to generate collagenase-isolated clusters.

Single cells derived from collagenase-isolated clusters by 0.25% trypsin and 1 mM EDTA (T/E) at 37 C for 15 minutes are seeded at 1×10⁵ per cm² in the 6-well plastic plate with or without coated Matrigel, which was prepared by adding 40 ul of 5% Matrigel per cm² 1 hour before use and cultured in ESCM containing 4 ng/ml bFGF and 10 ng/ml LIF in humidified 5% CO₂ with medium changed every 3 or 4 days.

Cells at 80% or 90% confluence are rendered single cells by T/E and serially expanded at the seeding density of 5×10³ cells per cm² for up to 12 passages.

MSCs expanded on coated Matrigel at passage 4 (P4) are reseeded in 3D Matrigel to generate P4/3D aggregates. P4/3D aggregates are administered to an individual with a chronic wound. The wound heals.

### Example 23

### Use of MSC as for the Treatment of Crohn's Disease

Amniotic membrane is digested with 2 mg/ml collagenase A in MESCM at 37 C for 18 hours under humidified 5% CO₂ to generate collagenase-isolated clusters.

Single cells derived from collagenase-isolated clusters by 0.25% trypsin and 1 mM EDTA (T/E) at 37 C for 15 minutes are seeded at 1×10⁵ per cm² in the 6-well plastic plate with or without coated Matrigel, which was prepared by adding 40 ul of 5% Matrigel per cm² 1 hour before use and cultured in ESCM containing 4 ng/ml bFGF and 10 ng/ml LIF in humidified 5% CO₂ with medium changed every 3 or 4 days.

Cells at 80% or 90% confluence are rendered single cells by T/E and serially expanded at the seeding density of 5×10³ cells per cm² for up to 12 passages.

MSCs expanded on coated Matrigel at passage 4 (P4) are reseeded in 3D Matrigel to generate P4/3D aggregates. P4/3D aggregates are infused into an individual with Crohn's Disease. The Crohn's Disease is treated.

### Example 24

### Clinical Trial for Crohn's Disease

Subjects will receive mesenchymal stromal cell therapy weekly by IV infusion for 4 weeks and will be assessed for 4 hours post infusion (2×10⁶/kg recipient weight; infused over 15 minutes)

Study Type: Interventional

### Study Design:

Allocation: Non-Randomized

Endpoint Classification: Safety/Efficacy Study

Intervention Model: Single Group Assignment

Masking: Open Label

Primary Purpose: Treatment

### Primary Outcome Measures:

Clinical response to MSC: Reduction of Crohn's disease Activity score by 100 points or more at six weeks post start of therapy

Colonoscopy and biopsy as well as clinical parameters used for the Crohn's disease activity will be undertaken at screening pre-therapy and at 6 weeks after start of therapy.

### Secondary Outcome Measures:

Incidence of infusional toxicity

Induction of remission—Crohn's disease activity index assessed as below 150

Improved quality of life—Increase in IBDQ and SF-36 scores measured at six weeks

Endoscopic improvement—Crohn's disease endoscopic improvement score will be measured at repeat endoscopy six weeks after start of treatment

### Eligibility

18 Years to 55 Years

Genders Eligible for Study: Both

### Inclusion Criteria:

Colonic or Small Bowel Crohn's Disease Based on Endoscopic Appearances and Histology

Refractory to induction with infliximab or adalimumab; or have lost response to these agents; or have had side effects precluding their further use

Where there has been loss of response to one of these agents, the other must be tried before being eligible

Crohn's disease activity score (CDAI) 250 or more.

C-reactive protein >10 mg/L

Surgery must have been offered to the subject (if appropriate) and declined

Signed informed consent

### Exclusion Criteria:

Active sepsis, perforating disease. Coexistent perianal fistulous disease is permitted, providing no co-existent infection within previous 4 weeks

Chronic stricturing disease in isolation

Coexistent CMV disease

Prior history of malignancy

Pregnant or unwilling to practice contraceptive therapy or breast feeding females

Last biologic therapy must be greater than 4 weeks prior, must be on stable corticosteroid dose for 14 days prior, during therapy and for 14 days after therapy, must be on stable immunomodulator dose (eg, azathioprine) for 14 days prior, during therapy and for 14 days after.

While preferred embodiments have been shown and described herein, it will be obvious to those skilled in the art that such embodiments are provided by way of example only. Numerous variations, changes, and substitutions may now occur. It should be understood that various alternatives to the embodiments described herein may be employed in practicing the described methods. It is intended that the following claims define the scope of the embodiments and that methods and structures within the scope of these claims and their equivalents be covered thereby.

1.-140. (canceled)

141. A method of isolating and expanding a plurality of multipotent cells, comprising: isolating and expanding at least one expanding multipotent cell in a culture comprising a suitable 3-dimensional substrate, to generate a population of expanded multipotent cells.

142. The method of claim 141, further comprising expanding at least one isolated multipotent cell in a culture comprising a suitable 2-dimensional substrate, to generate the at least one expanding multipotent cell.

143. The method of claim 142, further comprising isolating the at least one isolated multipotent cell from a tissue sample, to generate the isolated multipotent cell.

144. The method of claim 143, further comprising isolating the at least one isolated multipotent cell from cells and components of an extracellular matrix of the tissue sample.

145. The method of claim 143, wherein the tissue sample is placenta, umbilical cord, chorion, limbal tissue, conjunctiva, the skin, the oral mucosa, adipose tissue and/or a combination thereof.

146. The method of claim 143, wherein the tissue sample comprises extracellular matrix and basement membrane.

147. The method of claim 146, wherein the at least one isolated multipotent cell is not separated from the basement membrane.

148. The method of claim 146, further comprising contacting the tissue sample with an enzyme that degrades interstitial components of an extracellular matrix but not basement membrane components.

149. The method of claim 148, wherein the enzyme is a collagenase, dispase or a combination of collagenase and dispase.

150. The method of claim 149, wherein the collagenase is collagenase A.

151. The method of claim 141, wherein the at least one multipotent cell is selected from: mesenchymal stromal cells (MSC), adipose derived stromal cells (ASC), or a combination thereof.

152. The method of claim 141, wherein the 3-dimension substrate comprise a gelatinous protein mixture secreted by Engelbreth-Holm-Swarm (EHS) mouse sarcoma cells, laminin, type IV collagen, heparan sulfate proteoglycans, an inhibitor of Rho-associated kinase, or any combinations thereof.

153. The method of claim 142, wherein the 2-dimension substrate comprise a gelatinous protein mixture secreted by Engelbreth-Holm-Swarm (EHS) mouse sarcoma cells, laminin, type IV collagen, heparan sulfate proteoglycans, an inhibitor of Rho-associated kinase, or any combinations thereof.

154. The method of claim 141, wherein the culture comprising the 3-dimensional substrate comprises a culture medium selected from: embryonic stem cell medium, supplemented hormonal epithelial medium, or a combination thereof.

155. The method of claim 142, wherein the culture comprising the 2-dimensional substrate comprises a culture medium selected from: embryonic stem cell medium, supplemented hormonal epithelial medium, or a combination thereof.

156. The method of claim 154 or 155, wherein the embryonic stem cell medium is a human embryonic stem cell medium.

157. The method of claim 154 or 155, wherein the embryonic stem cell medium comprises basic fibroblast growth factor (bFGF), leukemia inhibitory factor (LIF), or a combination thereof.

158. An expanded multipotent cell culture obtained by the method of claim 141.
